(19) United States
(12) Patent Application Publication
(10) Pub. No.: US 2024/0425864 A1
(43) Pub. Date: Dec. 26, 2024

(54) ANTISENSE OLIGONUCLEOTIDES HAVING ONE OR MORE ABASIC UNITS

(71) Applicant: Sarepta Therapeutics, Inc., Cambridge, MA (US)

(72) Inventors: Ryan OLIVER, Cambridge, MA (US); Kevin KIM, Cambridge, MA (US); Meghan AHERN, Cambridge, MA (US)

(73) Assignee: Sarepta Therapeutics, Inc., Cambridge, MA (US)

(21) Appl. No.: 18/693,769

(22) PCT Filed: Sep. 28, 2022
(86) PCT No.: PCT/US2022/044995

§ 371 Date: Mar. 20, 2024

Related U.S. Application Data
(60) Provisional application No. 63/261,860, filed on Sep. 30, 2021.
(60) Provisional application No. 63/408,277, filed on Sep. 20, 2022.

(51) Int. Cl. C12N 15/113

(52) U.S. Cl. CPC C12N 15/1137; C12N 2310/14; C12N 2310/314; C12N 2310/332; C12N 2310/3513

(57) ABSTRACT

Provided herein are oligonucleotides, peptide-oligonucleotide-conjugates, and a targeting sequence complementary to a target region within intron 1 of a pre-mRNA of human acid alpha-glucosidase (GAA) gene having at least one purine and pyrimidine-free abasic subunit. Also provided herein are methods of treating a muscle disease, a viral infection, or a bacterial infection in a subject in need thereof, comprising administering to the subject oligonucleotides, peptides, and peptide-oligonucleotide-conjugates described herein.

### RELATED APPLICATIONS

This application claims the benefit of U.S. Provisional Application No. 63/408,277, filed Sep. 20, 2022 and U.S. Provisional Application No. 63/261,860 filed Sep. 30, 2021, the entire contents of which is hereby incorporated by reference in its entirety.

### BACKGROUND

Antisense technology provides a means for modulating the expression of one or more specific gene products, including alternative splice products, and is uniquely useful in a number of therapeutic, diagnostic, and research applications. The principle behind antisense technology is that an antisense compound, e.g., an oligonucleotide, which hybridizes to a target nucleic acid, modulates gene expression activities such as transcription, splicing, or translation through any one of a number of antisense mechanisms. The sequence specificity of antisense compounds makes them attractive as tools for target validation and gene functionalization, as well as therapeutics, to selectively modulate the expression of genes involved in disease.

Glycogen storage disease type II (GSD-II) (also known as Pompe disease, glycogenosis II, acid maltase deficiency (AMD) is an inherited autosomal recessive lysosomal storage disorder caused by deficiency of an enzyme called acid alpha-glucosidase (GAA). The role of GAA within the body is to break down glycogen. Reduced or absent levels of GAA activity lead to the accumulation of glycogen in the affected tissues, including the heart, skeletal muscles (including those involved with breathing), liver, and nervous system. This accumulation of glycogen is believed to cause progressive muscle weakness and respiratory insufficiency in individuals with GSD-II. GSD-II can occur in infants, toddlers, or adults, and the prognosis varies according to the time of onset and severity of symptoms. Clinically, GSD-II may manifest with a broad and continuous spectrum of severity ranging from severe (infantile) to milder late-onset adult form. The patients eventually die due to respiratory insufficiency. There is a good correlation between the severity of the disease and the residual acid alpha-glucosidase activity, the activity being 10-20% of normal in late-onset and less than 2% in early-onset forms of the disease. It is estimated that GSD-II affects approximately 5,000 to 10,000 people worldwide.

The most common mutation associated with the adult-onset form of the disease is IVS1-13T>G. Found in over two-thirds of adult-onset GSD-II patients, this mutation may confer a selective advantage in heterozygous individuals or is a very old mutation. The wide ethnic variation of adult-onset GSD-II individuals with this mutation argues against a common founder.

The GAA gene consists of 20 exons spanning some 20 kb. The 3.4 kb mRNA encodes a protein with a molecular weight of approximately 105 kD. The IVS1-13T>G mutation leads to the complete or partial loss of exon 2 (577 bases) which contains the initiation AUG codon.

Treatment for GSD-II has involved drug treatment strategies, dietary manipulations, and bone marrow transplantation without significant success. In recent years, enzyme replacement therapy (ERT) has provided new hope for GSD-II patients. For example, Myozyme®, a recombinant GAA protein drug, received approval for use in patients with GSD-II disease in 2006 in both the U.S. and Europe. Myozyme® depends on mannose-6-phosphates (M6P) on the surface of the GAA protein for delivery to lysosomes. The U.S. Food and Drug Administration has also approved Nexviazyme® (avalglucosidase alfa-ngpt) for the treatment of patients with late-onset Pompe disease. Nexviazyme is an enzyme replacement therapy (ERT) designed to specifically target the M6P receptor.

Antisense technology, used mostly for RNA down-regulation, recently has been adapted to alter the splicing process. Processing the primary gene transcripts (pre-mRNA) of many genes involves the removal of introns and the precise splicing of exons where a donor splice site is joined to an acceptor splice site. Splicing is a precise process, involving the coordinated recognition of donor and acceptor splice sites, and the branch point (upstream of the acceptor pre-site) with a balance of positive exon splice enhancers (predominantly located within the exon) and negative splice motifs (splice silencers are located predominantly in the introns).

Although significant progress has been made in the field of antisense technology, there remains a need in the art for oligonucleotides and peptide-oligonucleotide-conjugates with improved antisense or antigene performance for improved treatment of GSD-II.

### SUMMARY OF THE INVENTION

The present disclosure relates to antisense oligomers and related compositions and methods for inducing exon inclusion as a treatment for glycogen storage disease type II (GSD-II) (also known as Pompe disease, glycogenosis II, acid maltase deficiency (AMD), acid alpha-glucosidase deficiency, and lysosomal alpha-glucosidase deficiency), and more specifically relates to inducing inclusion of exon 2 and thereby restoring levels of enzymatically active acid alpha-glucosidase (GAA) protein encoded by the GAA gene.

Thus, provided herein are antisense oligomers or pharmaceutically acceptable salts thereof, wherein the antisense oligomer is 18-40 subunits in length, comprising a targeting sequence complementary to a target region within intron 1 (SEQ ID NO: 1) of a pre-mRNA of human acid alpha-glucosidase (GAA) gene, wherein:
each subunit of the antisense oligomer comprises a nucleobase or is an abasic subunit;
at least one subunit is an abasic subunit; and
wherein the targeting sequence, except for the abasic subunit or subunits, is at least 80% complementary to the target region.

The antisense oligomers are useful for the treatment for various diseases in a subject in need thereof, including, but not limited to, diseases such as Pompe Disease.

The antisense oligomer can be a phosphorodiamidate morpholino oligomer. The antisense oligomer can further comprise a cell-penetrating peptide. The peptide can be any of the peptides provided herein or known in the art.

In an embodiment, the target region comprises a sequence selected from the group consisting of SEQ ID NO: 2 (GAA-IVS1(-189-167)) and SEQ ID NO: 3 (GAA-IVS1(-80-24)). In another embodiment, the targeting region is selected from GAA-IVS1(-189-167), GAA-IVS1(-72,-48), GAA-IVS1(-71,-47), GAA-IVS1(-70,-46), GAA-IVS1(-69-45), GAA-IVS1(-65,-41), GAA-IVS1(-66,-42). In a further embodiment, the targeting region is GAA-IVS1(-189-167). In another embodiment, the targeting region is GAA-IVS1(-72,-48). In still another embodiment, the targeting region is GAA-IVS1(-71,-47). In yet another embodiment, the targeting region is GAA-IVS1(-70,-46). In an embodiment, the targeting region is GAA-IVS1(-69-45). In another embodiment, the targeting region is GAA-IVS1(-65,-41). In still another embodiment, the targeting region is GAA-IVS1(-66,-42).

In an embodiment, the targeting sequence comprises or consists of any one of the sequences:

| | Targeting Sequence | SEQ |
|---|---|---|
| Name | [5′ to 3′] | ID NO: |
| GAA-IVS1 | CCA GAA GGA AXX XCG AGA AAA GC | 4 |
| (−189,167) | | |
| GAA-IVS1 | CTC ACX XXX CTC TCA AAG CAG CTC T | 11 |
| (−72,−48) | | |
| GAA-IVS1 | ACT CAC XXX XCT CTC AAA GCA GCT C | 12 |
| (−71,−47) | | |
| GAA-IVS1 | CAC TCA CXX XXC TCT CAA AGC AGC T | 13 |
| (−70,−46) | | |
| GAA-IVS1 | GCA CTC ACX XXX CTC TCA AAG CAG C | 14 |
| (−69−45) | | |
| GAA-IVS1 | GCG GCA CTC ACX XXX CTC TCA AAG C | 15 |
| (−65,−41) | | |
| GAA-IVS1 | GGC GGC ACT CAC XXX XCT CTC AAA G | 16 |
| (−66,−42) | | |

wherein each X is independently selected from guanine (G) or is abasic (B), wherein at least one X is B. In instances where X is abasic (B), hydrogen is present in place of nucleobases A, C, T, or G.

In an embodiment, B is H.

In a further embodiment, the targeting sequence comprises or consists of any one of the sequences:

| | Targeting Sequence | SEQ |
|---|---|---|
| Name | [5′ to 3′] | ID NO: |
| GAA-IVS1(−189, 167) | CCA GAA GGA AGG BCG | 5 |
| (−179 Abasic) | AGA AAA GC | |
| GAA-IVS1(−189, 167) | CCA GAA GGA AGB GCG | 6 |
| (−178 Abasic) | AGA AAA GC | |
| GAA-IVS1(−189, 167) | CCA GAA GGA ABG GCG | 7 |
| (−177 Abasic) | AGA AAA GC | |
| GAA-IVS1(−189, 167) | CCA GAA GGA AGB BCG | 8 |
| (−178,−179 Abasic) | AGA AAA GC | |
| GAA-IVS1(−189, 167) | CCA GAA GGA ABB GCG | 9 |
| (−177,−178 Abasic) | AGA AAA GC | |
| GAA-IVS1(−189, 167) | CCA GAA GGA ABG BCG | 10 |
| (−177,−179 Abasic) | AGA AAA GC | |
| GAA-IVS1(−69−45) | GCA CTC ACB GGG CTC | 17 |
| (G53B) | TCA AAG CAG C | |
| GAA-IVS1(−69−45) | GCA CTC ACG BGG CTC | 18 |
| (G54B) | TCA AAG CAG C | |
| GAA-IVS1(−69−45) | GCA CTC ACG GBG CTC | 19 |
| (G55B) | TCA AAG CAG C | |
| GAA-IVS1(−69−45) | GCA CTC ACG GGB CTC | 20 |
| (G56B) | TCA AAG CAG C | |
| GAA-IVS1(−69−45) | GCA CTC ACB BGG CTC | 21 |
| (G53B G54B) | TCA AAG CAG C | |
| GAA-IVS1(−69−45) | GCA CTC ACG BBG CTC | 22 |
| (G54B G55B) | TCA AAG CAG C | |
| GAA-IVS1(−69−45) | GCA CTC ACG GBB CTC | 23 |
| (G55B G56B) | TCA AAG CAG C | |
| GAA-IVS1(−65−41) | GGC GGC ACT CAC GBB | 24. |
| (G54B G55B) | GCT CTC AAA G | |

In an embodiment, B is H.

In some aspects, the nucleobases of the antisense oligomer are linked to morpholino ring structures, wherein the morpholino ring structures are joined by phosphorous-containing intersubunit linkages joining a morpholino nitrogen of one ring structure to a 5′ exocyclic carbon of an adjacent ring structure.

In some aspects, the nucleobases of the antisense oligomer are linked to a peptide nucleic acid (PNA), wherein the phosphate-sugar polynucleotide backbone is replaced by a flexible pseudo-peptide polymer to which the nucleobases are linked.

In some aspects, at least one of the nucleobases of the antisense oligomer is linked to a locked nucleic acid (LNA), wherein the locked nucleic acid structure is a nucleotide analog that is chemically modified where the ribose moiety has an extra bridge connecting the 2′ oxygen and the 4′ carbon.

In some aspects, at least one of the nucleobases of the antisense oligomer is linked to a bridged nucleic acid (BNA), wherein the sugar conformation is restricted or locked by the introduction of an additional bridged structure to the furanose skeleton. In some aspects, at least one of the nucleobases of the antisense oligomer is linked to a 2′-O,4′-C-ethylene-bridged nucleic acid (ENA).

In some aspects, the modified antisense oligomer may contain unlocked nucleic acid (UNA) subunits. UNAs and UNA oligomers are an analogue of RNA in which the C2′-C3′ bond of the subunit has been cleaved.

In some aspects, the modified antisense oligomer contains one or more phosphorothioates (or S-oligos), in which one of the nonbridging oxygens is replaced by a sulfur. In some aspects, the modified antisense oligomer contains one or more 2′ O-Methyl, 2′ O-MOE, MCE, and 2′-F in which the 2′-OH of the ribose is substituted with a methyl, methoxyethyl, 2-(N-methylcarbamoyl)ethyl, or fluoro group, respectively.

In some aspects, the modified antisense oligomer is a tricyclo-DNA (tc-DNA) which is a constrained DNA analog in which each nucleotide is modified by the introduction of a cyclopropane ring to restrict conformational flexibility of the backbone and to optimize the backbone geometry of the torsion angle γ.

In an aspect, the antisense oligomer is a modified antisense oligonucleotide, wherein:
the modified antisense oligonucleotide is 18-40 subunits in length, comprising a targeting sequence complementary to a target region within intron 1 (SEQ ID NO: 1) of a pre-mRNA of human acid alpha-glucosidase (GAA) gene, wherein:
the antisense oligonucleotide comprises a morpholino oligomer;
each subunit of the antisense oligonucleotide comprises a nucleobase or is an abasic subunit, wherein each subunit is taken together in order from the 5′ end of the antisense oligonucleotide to the 3′ end of the antisense oligonucleotide form the targeting sequence;
at least one subunit is an abasic subunit; and
wherein the targeting sequence, except for the abasic subunit or subunits, is at least 80% complementary to the target region.

In an embodiment, the disclosure provides antisense oligomers according to Formula I:

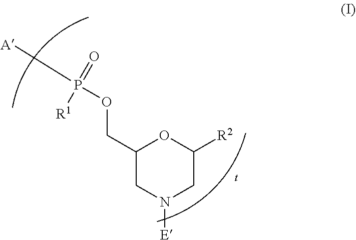

or a pharmaceutically acceptable salt thereof,
wherein:
A′ is selected from —N(H)CH₂C(O)NH₂, —N(C₁₋₆-alkyl)CH₂C(O)NH₂,

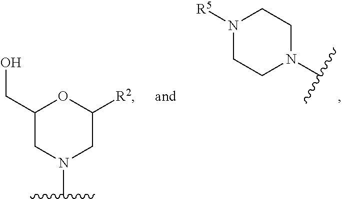

wherein
R⁵ is —C(O)(O-alkyl)ₓ-OH, wherein x is 3-10 and each alkyl group is, independently at each occurrence, C₂₋₆-alkyl,
or R⁵ is selected from H, —C(O)C₁₋₆-alkyl, trityl, monomethoxytrityl, —(C₁₋₆-alkyl)-R⁶, —(C₁₋₆-heteroalkyl)-R⁶, aryl-R⁶, heteroaryl-R⁶, —C(O)O—(C₁₋₆-alkyl)-R⁶, —C(O)O-aryl-R⁶, —C(O)O-heteroaryl-R⁶, and

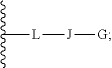

R⁶ is selected from OH, SH, and NH₂, or R⁶ is O, S, or NH, each of which is covalently linked to a solid support;
each R¹ is independently selected from OH and —N(R³)(R⁴), wherein each R³ and R⁴ are, independently at each occurrence, H or —C₁₋₆-alkyl;
each R² is independently, at each occurrence, selected from H (abasic), a nucleobase, and a nucleobase functionalized with a chemical protecting group, wherein the nucleobase, independently at each occurrence, comprises a C₃₋₆-heterocyclic ring selected from pyridine, pyrimidine, purine, and deaza-purine;
t is 8-40;
E′ is selected from H, —C₁₋₆-alkyl, —C(O)C₁₋₆-alkyl, benzoyl, stearoyl, trityl, monomethoxytrityl, dimethoxytrityl, trimethoxytrityl,

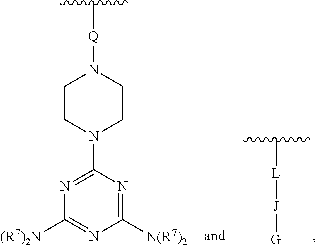

wherein
   Q is —C(O)(CH₂)₆C(O)— or —C(O)(CH₂)₂S₂(CH₂)₂C(O)—;
R⁷ is —(CH₂)₂OC(O)N(R⁸)₂, wherein R⁸ is —(CH₂)₆NHC(═NH)NH₂;
L is selected from glycine, proline, W, W—W, or R⁹, wherein L is covalently linked by an amide bond to the N-terminus or C-terminus of J;
W is —C(O)—(CH₂)ₘ—NH—, wherein m is 2 to 12;
R⁹ is selected from the group consisting of:

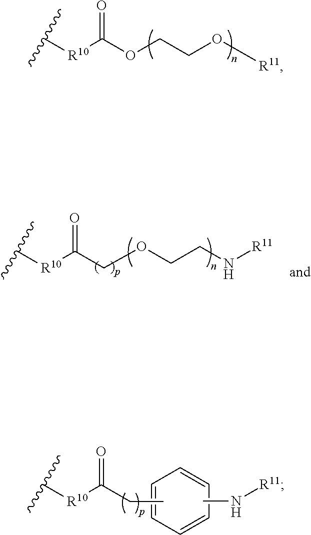

n is 1, 2, 3, 4, 5, 6, 7, 8, 9, or 10;
p is 2, 3, 4, or 5;
R¹⁰ is selected from a bond, glycine, proline, W, or W—W;
R¹¹ is selected from the group consisting of glycine, proline, W, W—W, and

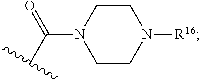

R¹⁶ is selected from a bond, glycine, proline, W, or W—W; wherein R¹⁶ is covalently linked by an amide bond to the N-terminus or C-terminus of J; J is a cell-penetrating peptide; and
G is selected from H, —C(O)C₁₋₆-alkyl, benzoyl, and stearoyl, wherein G is covalently linked to J.

In some embodiments, an antisense oligomer of the disclosure is according to Formula II:

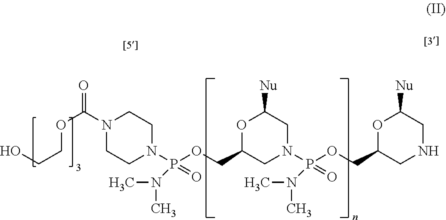

or a pharmaceutically acceptable salt thereof, where each Nu from 1 to n and 5′ to 3′ corresponds to the nucleobases in one of the following:

| | Targeting Sequence | SEQ |
|---|---|---|
| Name | [5′ to 3′] | ID NO: |
| GAA-IVS1 | CCA GAA GGA AXX XCG AGA AAA GC | 4 |
| (−189,167) | | |
| GAA-IVS1 | CTC ACX XXX CTC TCA AAG CAG CTC T | 11 |
| (−72,−48) | | |
| GAA-IVS1 | ACT CAC XXX XCT CTC AAA GCA GCT C | 12 |
| (−71,−47) | | |
| GAA-IVS1 | CAC TCA CXX XXC TCT CAA AGC AGC T | 13 |
| (−70,−46) | | |
| GAA-IVS1 | GCA CTC ACX XXX CTC TCA AAG CAG C | 14 |
| (−69−45) | | |
| GAA-IVS1 | GCG GCA CTC ACX XXX CTC TCA AAG C | 15 |
| (−65,−41) | | |
| GAA-IVS1 | GGC GGC ACT CAC XXX XCT CTC AAA G | 16 |
| (−66,−42) | | |

wherein each X is independently selected from guanine (G) or is abasic (B), wherein at least one X is B. In instances where X is abasic (B), hydrogen is present in place of nucleobases A, C, T, or G.

In an embodiment, B is H.

In an embodiment, the targeting sequence comprises or consists of any one of the sequences:

| | Targeting Sequence | SEQ |
|---|---|---|
| Name | [5′ to 3′] | ID NO: |
| GAA-IVS1(−189, 167) | CCA GAA GGA AGG BCG | 5 |
| (−179 Abasic) | AGA AAA GC | |
| GAA-IVS1(−189, 167) | CCA GAA GGA AGB GCG | 6 |
| (−178 Abasic) | AGA AAA GC | |
| GAA-IVS1(−189, 167) | CCA GAA GGA ABG GCG | 7 |
| (−177 Abasic) | AGA AAA GC | |
| GAA-IVS1(−189, 167) | CCA GAA GGA AGB BCG | 8 |
| (−178,−179 Abasic) | AGA AAA GC | |
| GAA-IVS1(−189, 167) | CCA GAA GGA ABB GCG | 9 |
| (−177,−178 Abasic) | AGA AAA GC | |
| GAA-IVS1(−189, 167) | CCA GAA GGA ABG BCG | 10 |
| (−177,−179 Abasic) | AGA AAA GC | |
| GAA-IVS1(−69−45) | GCA CTC ACB GGG CTC | 17 |
| (G53B) | TCA AAG CAG C | |
| GAA-IVS1(−69−45) | GCA CTC ACG BGG CTC | 18 |
| (G54B) | TCA AAG CAG C | |
| GAA-IVS1(−69−45) | GCA CTC ACG GBG CTC | 19 |
| (G55B) | TCA AAG CAG C | |
| GAA-IVS1(−69−45) | GCA CTC ACG GGB CTC | 20 |
| (G56B) | TCA AAG CAG C | |
| GAA-IVS1(−69−45) | GCA CTC ACB BGG CTC | 21 |
| (G53B G54B) | TCA AAG CAG C | |
| GAA-IVS1(−69−45) | GCA CTC ACG BBG CTC | 22 |
| (G54B G55B) | TCA AAG CAG C | |
| GAA-IVS1(−69−45) | GCA CTC ACG GBB CTC | 23 |
| (G55B G56B) | TCA AAG CAG C | |
| GAA-IVS1(−65−41) | GGC GGC ACT CAC GBB | 24. |
| (G54B G55B) | GCT CTC AAA G | |

In an embodiment, B is H.

In an embodiment, the antisense oligomer is a conjugate comprising a modified antisense oligonucleotide and a cell-penetrating peptide, wherein:
the modified antisense oligonucleotide is 18-40 subunits in length, comprising a targeting sequence complementary to a target region within intron 1 (SEQ ID NO: 1) of a pre-mRNA of human acid alpha-glucosidase (GAA) gene, wherein:
the antisense oligonucleotide comprises a morpholino oligomer;
the antisense oligonucleotide is covalently linked to the cell-penetrating peptide;
each subunit of the antisense oligonucleotide comprises a nucleobase or is an abasic subunit, wherein each subunit is taken together in order from the 5′ end of the antisense oligonucleotide to the 3′ end of the antisense oligonucleotide form the targeting sequence;
at least one subunit is an abasic subunit; and
wherein the targeting sequence, except for the abasic subunit or subunits, is at least 80% complementary to the target region.

Thus, in an embodiment of Formula I, or a salt thereof, A′ is

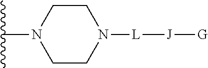

### or E′ is

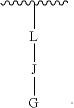

In some embodiments, an antisense oligomer of the disclosure is according to Formula (IIIa):

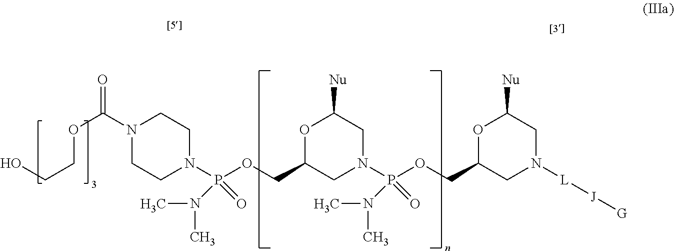

or a pharmaceutically acceptable salt thereof, where each Nu from 1 to n and 5′ to 3′ corresponds to the nucleobases in one of the following:

| | Targeting Sequence | SEQ |
|---|---|---|
| Name | [5′ to 3′] | ID NO: |
| GAA-IVS1 | CCA GAA GGA AXX XCG AGA AAA GC | 4 |
| (−189,167) | | |
| GAA-IVS1 | CTC ACX XXX CTC TCA AAG CAG CTC T | 11 |
| (−72,−48) | | |
| GAA-IVS1 | ACT CAC XXX XCT CTC AAA GCA GCT C | 12 |
| (−71,−47) | | |
| GAA-IVS1 | CAC TCA CXX XXC TCT CAA AGC AGC T | 13 |
| (−70,−46) | | |
| GAA-IVS1 | GCA CTC ACX XXX CTC TCA AAG CAG C | 14 |
| (−69−45) | | |
| GAA-IVS1 | GCG GCA CTC ACX XXX CTC TCA AAG C | 15 |
| (−65,−41) | | |
| GAA-IVS1 | GGC GGC ACT CAC XXX XCT CTC AAA G | 16. |
| (−66,−42) | | |

wherein each X is independently selected from guanine (G) or is abasic (B), wherein at least one X is B. In instances where X is abasic (B), hydrogen is present in place of nucleobases A, C, T, or G.

In an embodiment, B is H.

In an embodiment, the targeting sequence comprises or consists of any one of the sequences:

| | Targeting Sequence | SEQ |
|---|---|---|
| Name | [5′ to 3′] | ID NO: |
| GAA-IVS1(−189, 167) | CCA GAA GGA AGG BCG | 5 |
| (−179 Abasic) | AGA AAA GC | |
| GAA-IVS1(−189, 167) | CCA GAA GGA AGB GCG | 6 |
| (−178 Abasic) | AGA AAA GC | |
| GAA-IVS1(−189, 167) | CCA GAA GGA ABG GCG | 7 |
| (−177 Abasic) | AGA AAA GC | |
| GAA-IVS1(−189, 167) | CCA GAA GGA AGB BCG | 8 |
| (−178,−179 Abasic) | AGA AAA GC | |
| GAA-IVS1(−189, 167) | CCA GAA GGA ABB GCG | 9 |
| (−177,−178 Abasic) | AGA AAA GC | |
| GAA-IVS1(−189, 167) | CCA GAA GGA ABG BCG | 10 |
| (−177,−179 Abasic) | AGA AAA GC | |
| GAA-IVS1(−69−45) | GCA CTC ACB GGG CTC | 17 |
| (G53B) | TCA AAG CAG C | |
| GAA-IVS1(−69−45) | GCA CTC ACG BGG CTC | 18 |
| (G54B) | TCA AAG CAG C | |
| GAA-IVS1(−69−45) | GCA CTC ACG GBG CTC | 19 |
| (G55B) | TCA AAG CAG C | |
| GAA-IVS1(−69−45) | GCA CTC ACG GGB CTC | 20 |
| (G56B) | TCA AAG CAG C | |
| GAA-IVS1(−69−45) | GCA CTC ACB BGG CTC | 21 |
| (G53B G54B) | TCA AAG CAG C | |
| GAA-IVS1(−69−45) | GCA CTC ACG BBG CTC | 22 |
| (G54B G55B) | TCA AAG CAG C | |
| GAA-IVS1(−69−45) | GCA CTC ACG GBB CTC | 23 |
| (G55B G56B) | TCA AAG CAG C | |
| GAA-IVS1(−65−41) | GGC GGC ACT CAC GBB | 24. |
| (G54B G55B) | GCT CTC AAA G | |

In an embodiment, B is H.

In some embodiments, an antisense oligomer of the disclosure is according to Formula (III):

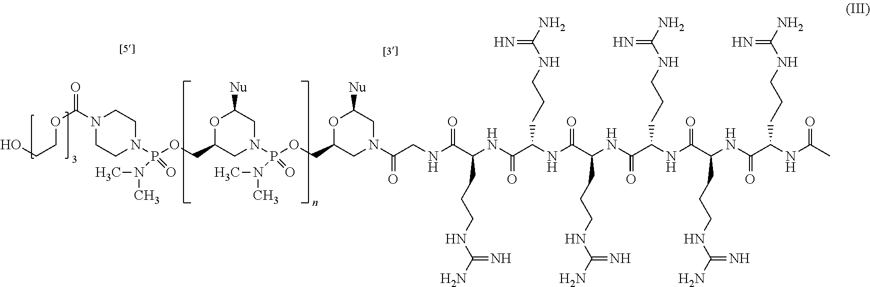

or a pharmaceutically acceptable salt thereof, where each Nu from 1 to n and 5′ to 3′ corresponds to the nucleobases in one of the following:

| | Targeting Sequence | SEQ |
|---|---|---|
| Name | [5′ to 3′] | ID NO: |
| GAA-IVS1 | CCA GAA GGA AXX XCG AGA AAA GC | 4 |
| (−189,167) | | |
| GAA-IVS1 | CTC ACX XXX CTC TCA AAG CAG CTC T | 11 |
| (−72,−48) | | |
| GAA-IVS1 | ACT CAC XXX XCT CTC AAA GCA GCT C | 12 |
| (−71,−47) | | |
| GAA-IVS1 | CAC TCA CXX XXC TCT CAA AGC AGC T | 13 |
| (−70,−46) | | |
| GAA-IVS1 | GCA CTC ACX XXX CTC TCA AAG CAG C | 14 |
| (−69−45) | | |
| GAA-IVS1 | GCG GCA CTC ACX XXX CTC TCA AAG C | 15 |
| (−65,−41) | | |
| GAA-IVS1 | GGC GGC ACT CAC XXX XCT CTC AAA G | 16 |
| (−66,−42) | | |

wherein each X is independently selected from guanine (G) or is abasic (B), wherein at least one X is B. In instances where X is abasic (B), hydrogen is present in place of nucleobases A, C, T, or G.

In an embodiment, B is H.

In an embodiment, the targeting sequence comprises or consists of any one of the sequences:

| | Targeting Sequence | SEQ |
|---|---|---|
| Name | [5′ to 3′] | ID NO: |
| GAA-IVS1(−189, 167) | CCA GAA GGA AGG BCG | 5 |
| (−179 Abasic) | AGA AAA GC | |
| GAA-IVS1(−189, 167) | CCA GAA GGA AGB GCG | 6 |
| (−178 Abasic) | AGA AAA GC | |
| GAA-IVS1(−189, 167) | CCA GAA GGA ABG GCG | 7 |
| (−177 Abasic) | AGA AAA GC | |
| GAA-IVS1(−189, 167) | CCA GAA GGA AGB BCG | 8 |
| (−178,−179 Abasic) | AGA AAA GC | |
| GAA-IVS1(−189, 167) | CCA GAA GGA ABB GCG | 9 |
| (−177,−178 Abasic) | AGA AAA GC | |
| GAA-IVS1(−189, 167) | CCA GAA GGA ABG BCG | 10 |
| (−177,−179 Abasic) | AGA AAA GC | |
| GAA-IVS1(−69−45) | GCA CTC ACB GGG CTC | 17 |
| (G53B) | TCA AAG CAG C | |
| GAA-IVS1(−69−45) | GCA CTC ACG BGG CTC | 18 |
| (G54B) | TCA AAG CAG C | |
| GAA-IVS1(−69−45) | GCA CTC ACG GBG CTC | 19 |
| (G55B) | TCA AAG CAG C | |
| GAA-IVS1(−69−45) | GCA CTC ACG GGB CTC | 20 |
| (G56B) | TCA AAG CAG C | |
| GAA-IVS1(−69−45) | GCA CTC ACB BGG CTC | 21 |
| (G53B G54B) | TCA AAG CAG C | |
| GAA-IVS1(−69−45) | GCA CTC ACG BBG CTC | 22 |
| (G54B G55B) | TCA AAG CAG C | |
| GAA-IVS1(−69−45) | GCA CTC ACG GBB CTC | 23 |
| (G55B G56B) | TCA AAG CAG C | |
| GAA-IVS1(−65−41) | GGC GGC ACT CAC GBB | 24. |
| (G54B G55B) | GCT CTC AAA G | |

In an embodiment, B is H.

The antisense oligomer can promote retention of exon 2 in the GAA mRNA upon binding of the targeting sequence to the target region. The antisense oligomer retains the potency of GAA enzyme activity compared to a second antisense oligonucleotide that is fully complementary to the target region within SEQ ID NO: 1. In another aspect, provided herein is a pharmaceutical composition comprising an antisense oligomer provided herein and a pharmaceutically acceptable carrier.

Also provided herein is a method of treating a disease comprising administering to a subject a therapeutically effective amount of the antisense oligomer provided herein.

In some embodiments, the antisense oligomer as described herein can be used for treating Pompe disease.

### BRIEF DESCRIPTION OF THE FIGURES

FIG. 1 shows a bar graph depicting GAA enzyme activity (Enzyme Assay) found for various PMO compounds during screening. The Y-axis represents the fold increase in GAA enzyme activity relative to untreated control. Individual compounds were dosed at 10 μM.

FIG. 2A shows a bar graph depicting GAA enzyme activity (Enzyme Assay) found for various PMO compounds during screening. The Y-axis represents the enzyme activity (mmol/mg hr) and fold increase in GAA enzyme activity relative to untreated control (UT).

FIG. 2B shows a bar graph depicting GAA enzyme activity (Enzyme Assay) found for various PMO compounds during screening. The Y-axis represents the fold increase in GAA enzyme activity relative to untreated control. Individual compounds were dosed at 20 μM.

FIG. 3 shows bar graphs depicting antisense microwalk data at the −65 region of intron 1 of a pre-mRNA of human acid alpha-glucosidase (GAA) gene. Individual compounds were dosed at 20 μM.

FIG. 4A shows a bar graph depicting GAA enzyme activity (Enzyme Assay) found for various PPMO compounds during screening. The X-axis represents the fold increase in GAA enzyme activity relative to non-targeting control. Individual compounds were dosed at 20 μM.

FIG. 4B shows a bar graph depicting GAA mRNA transcript levels (qPCR assay) found for various PPMO compounds during screening. The X-axis represents the fold increase in GAA mRNA transcript as measured at two locations within the GAA mRNA relative to non-targeting and untreated controls. Individual compounds were dosed at 30 μM.

FIG. 5 shows bar graphs depicting antisense microwalk data at the −169 region of intron 1 of a pre-mRNA of GAA gene. Individual compounds were dosed at 10 μM.

FIG. 6 shows a graph depicting dose dependent increases of GAA enzyme activity in patient fibroblasts after gymnotic treatment with PPMOs #33, 34, 5, and 7.

FIG. 7 shows a bar graph depicting GAA mRNA transcript levels (qPCR assay) found for PPMO compounds during screening. The Y-axis represents the fold increase in GAA mRNA transcript relative to non-targeting and untreated controls. Individual compounds were dosed at 1, 2.5, 5, 10, 20, and 30 μM.

FIG. 8 shows a graph depicting dose dependent increases of GAA expression measured across the Exon 1-2 junction in patient iPSC-derived myotubes after gymnotic treatment with select PPMOs #34, 5, and 7.

FIG. 9 shows digital gel images and graphs depicting increases in the amount of GAA protein normalized to the total protein in patient iPSC-derived myotubes after treatment with PPMOs #5, 7, and 34.

FIG. 10 shows a digital gel image and graph depicting increases in the amount of GAA protein normalized to the total protein in patient iPSC-derived myotubes after treatment with PPMOs #12 and 15.

FIG. 11 shows a graph depicting increases in the amount of GAA enzyme activity in patient iPSC-derived myotubes after treatment with PPMOs #7, 5, and 12.

FIG. 12 shows graphs depicting the aggreatation potential of selected PPMO compounds. Results are plotted as size/intensity distribution.

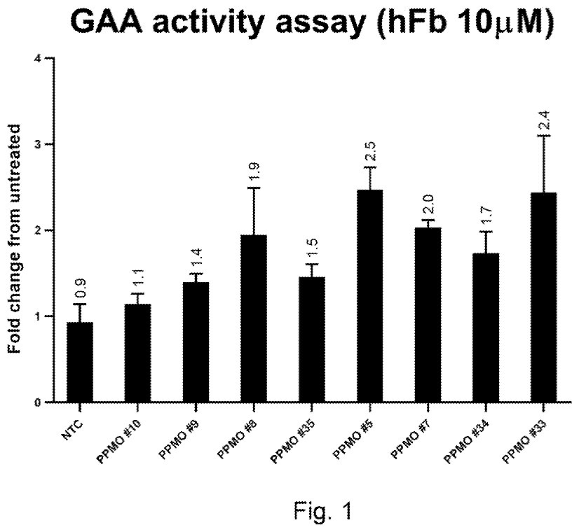

### DETAILED DESCRIPTION OF THE INVENTION

Provided herein are antisense oligomers or pharmaceutically acceptable salts thereof, wherein the antisense oligomer is 18-40 subunits in length, comprising a targeting sequence complementary to a target region within intron 1 (SEQ ID NO: 1) of a pre-mRNA of human acid alpha-glucosidase (GAA) gene, wherein at least one subunit is an abasic subunit. The antisense oligomers are useful for the treatment of various diseases in a subject in need thereof, including, but not limited to, Pompe disease.

Certain embodiments relate to methods for enhancing the level of exon 2-containing GAA-coding mRNA relative to exon-2 deleted GAA mRNA in a cell, comprising contacting the cell with an antisense oligomer of sufficient length and complementarity to specifically hybridize to a region within the GAA gene, such that the level of exon 2-containing GAA mRNA relative to exon-2 deleted GAA mRNA in the cell is enhanced. In some embodiments, the cell is in a subject, and the method comprises administering the antisense oligomer to the subject.

In an embodiment, provided herein are antisense oligomers comprising a cell-penetrating peptide, wherein the antisense oligomer comprises a targeting sequence complementary to a target region within intron 1 (SEQ ID NO: 1) of a pre-mRNA of human acid alpha-glucosidase (GAA) gene, and at least one subunit is an abasic subunit.

Also provided herein is a method for treating Pompe disease.

### I. Definitions

Unless defined otherwise, all technical and scientific terms used herein have the same meaning as commonly understood by those of ordinary skill in the art to which the disclosure belongs. Although any methods and materials similar or equivalent to those described herein can be used in the practice or testing of the subject matter of the present disclosure, preferred methods and materials are described. For the purposes of the present disclosure, the following terms are defined below.

The term “about” will be understood by persons of ordinary skill in the art and will vary to some extent on the context in which it is used. As used herein when referring to a measurable value such as an amount, a temporal duration, and the like, the term “about” is meant to encompass variations of ±10%, including ±5%, ±1%, and ±0.1% from the specified value, as such variations are appropriate to perform the disclosed methods.

The term “alkyl” refers to saturated, straight- or branched-chain hydrocarbon moieties containing, in certain embodiments, between one and six, or one and eight carbon atoms, respectively. Examples of C₁₋₆-alkyl moieties include, but are not limited to, methyl, ethyl, propyl, isopropyl, n-butyl, tert-butyl, neopentyl, n-hexyl moieties; and examples of C₁₋₈-alkyl moieties include, but are not limited to, methyl, ethyl, propyl, isopropyl, n-butyl, tert-butyl, neopentyl, n-hexyl, heptyl, and octyl moieties.

The number of carbon atoms in an alkyl substituent can be indicated by the prefix “C_{x-y},” where x is the minimum and y is the maximum number of carbon atoms in the substituent. Likewise, a Cₓ chain means an alkyl chain containing x carbon atoms.

The term “heteroalkyl” by itself or in combination with another term means, unless otherwise stated, a stable straight or branched chain alkyl group consisting of the stated number of carbon atoms and one or two heteroatoms selected from the group consisting of O, N, and S, and wherein the nitrogen and sulfur atoms may be optionally oxidized and the nitrogen heteroatom may be optionally quaternized. The heteroatom(s) may be placed at any position of the heteroalkyl group, including between the rest of the heteroalkyl group and the fragment to which it is attached, as well as attached to the most distal carbon atom in the heteroalkyl group. Examples include: —O—CH₂—CH₂—CH₃, —CH₂—CH₂—CH₂—OH, —CH₂—CH₂—NH—CH₃, —CH₂—S—CH₂—CH₃, and —CH₂—CH₂—S(═O)—CH₃. Up to two heteroatoms may be consecutive, such as, for example, —CH₂—NH—OCH₃, or —CH₂—CH₂—S—S—CH₃.

The term “aryl,” employed alone or in combination with other terms, means, unless otherwise stated, a carbocyclic aromatic system containing one or more rings (typically one, two, or three rings), wherein such rings may be attached together in a pendent manner, such as a biphenyl, or may be fused, such as naphthalene. Examples of aryl groups include phenyl, anthracyl, and naphthyl. In various embodiments, examples of an aryl group may include phenyl (e.g., C₆-aryl) and biphenyl (e.g., C₁₂-aryl). In some embodiments, aryl groups have from six to sixteen carbon atoms. In some embodiments, aryl groups have from six to twelve carbon atoms (e.g., C₆₋₁₂-aryl). In some embodiments, aryl groups have six carbon atoms (e.g., C₆-aryl).

As used herein, the term “heteroaryl” or “heteroaromatic” refers to a heterocycle having aromatic character. Heteroaryl substituents may be defined by the number of carbon atoms, e.g., C₁₋₉-heteroaryl indicates the number of carbon atoms contained in the heteroaryl group without including the number of heteroatoms. For example, a C₁₋₉-heteroaryl will include an additional one to four heteroatoms. A polycyclic heteroaryl may include one or more rings that are partially saturated. Non-limiting examples of heteroaryls include pyridyl, pyrazinyl, pyrimidinyl (including, e.g., 2- and 4-pyrimidinyl), pyridazinyl, thienyl, furyl, pyrrolyl (including, e.g., 2-pyrrolyl), imidazolyl, thiazolyl, oxazolyl, pyrazolyl (including, e.g., 3- and 5-pyrazolyl), isothiazolyl, 1,2,3-triazolyl, 1,2,4-triazolyl, 1,3,4-triazolyl, tetrazolyl, 1,2,3-thiadiazolyl, 1,2,3-oxadiazolyl, 1,3,4-thiadiazolyl and 1,3,4-oxadiazolyl.

Non-limiting examples of polycyclic heterocycles and heteroaryls include indolyl (including, e.g., 3-, 4-, 5-, 6- and 7-indolyl), indolinyl, quinolyl, tetrahydroquinolyl, isoquinolyl (including, e.g., 1- and 5-isoquinolyl), 1,2,3,4-tetrahydroisoquinolyl, cinnolinyl, quinoxalinyl (including, e.g., 2- and 5-quinoxalinyl), quinazolinyl, phthalazinyl, 1,8-naphthyridinyl, 1,4-benzodioxanyl, coumarin, dihydrocoumarin, 1,5-naphthyridinyl, benzofuryl (including, e.g., 3-, 4-, 5-, 6- and 7-benzofuryl), 2,3-dihydrobenzofuryl, 1,2-benzisoxazolyl, benzothienyl (including, e.g., 3-, 4-, 5-, 6-, and 7-benzothienyl), benzoxazolyl, benzothiazolyl (including, e.g., 2-benzothiazolyl and 5-benzothiazolyl), purinyl, benzimidazolyl (including, e.g., 2-benzimidazolyl), benzotriazolyl, thioxanthinyl, carbazolyl, carbolinyl, acridinyl, pyrrolizidinyl, and quinolizidinyl.

The term “protecting group” or “chemical protecting group” refers to chemical moieties that block some or all reactive moieties of a compound and prevent such moieties from participating in chemical reactions until the protective group is removed, for example, those moieties listed and described in T. W. Greene, P. G. M. Wuts, Protective Groups in Organic Synthesis, 3rd ed. John Wiley & Sons (1999). It may be advantageous, where different protecting groups are employed, that each (different) protective group be removable by a different means. Protective groups that are cleaved under totally disparate reaction conditions allow differential removal of such protecting groups. For example, protective groups can be removed by acid, base, and hydrogenolysis. Groups such as trityl, monomethoxytrityl, dimethoxytrityl, acetal and tert-butyldimethylsilyl are acid labile and may be used to protect carboxy and hydroxy reactive moieties in the presence of amino groups protected with Cbz groups, which are removable by hydrogenolysis, and Fmoc groups, which are base labile. Carboxylic acid moieties may be blocked with base labile groups such as, without limitation, methyl, or ethyl, and hydroxy reactive moieties may be blocked with base labile groups such as acetyl in the presence of amines blocked with acid labile groups such as tert-butyl carbamate or with carbamates that are both acid and base stable but hydrolytically removable.

Carboxylic acid and hydroxyl reactive moieties may also be blocked with hydrolytically removable protective groups such as the benzyl group, while amine groups may be blocked with base labile groups such as Fmoc. A particularly useful amine protecting group for the synthesis of compounds of Formula I and Formula IV is trifluoroacetamide. Carboxylic acid reactive moieties may be blocked with oxidatively-removable protective groups such as 2,4-dimethoxybenzyl, while coexisting amino groups may be blocked with fluoride labile silyl carbamates.

Allyl blocking groups are useful in the presence of acid- and base-protecting groups since the former are stable and can be subsequently removed by metal or pi-acid catalysts. For example, an allyl-blocked carboxylic acid can be deprotected with a palladium(0)-catalyzed reaction in the presence of acid labile t-butyl carbamate or base-labile acetate amine protecting groups. Yet another form of protecting group is a resin to which a compound or intermediate may be attached. As long as the residue is attached to the resin, that functional group is blocked and cannot react. Once released from the resin, the functional group is available to react.

The term “nucleobase,” “base pairing moiety,” “nucleobase-pairing moiety,” or “base” refers to the heterocyclic ring portion of a nucleoside, nucleotide, and/or morpholino subunit. Nucleobases may be naturally occurring (e.g., uracil, thymine, adenine, cytosine, and guanine), or may be modified or analogs of these naturally occurring nucleobases, e.g., one or more nitrogen atoms of the nucleobase may be independently at each occurrence replaced by carbon. Exemplary analogs include hypoxanthine (the base component of the nucleoside inosine); 2, 6-diaminopurine; 5-methyl cytosine; C5-propynyl-modified pyrimidines; 10-(9-(aminoethoxy)phenoxazinyl) (G-clamp) and the like.

Further examples of base pairing moieties include, but are not limited to, uracil, thymine, adenine, cytosine, guanine and hypoxanthine having their respective amino groups protected by acyl protecting groups, 2-fluorouracil, 2-fluorocytosine, 5-bromouracil, 5-iodouracil, 2,6-diaminopurine, azacytosine, pyrimidine analogs such as pseudoisocytosine and pseudouracil and other modified nucleobases such as 8-substituted purines, xanthine, or hypoxanthine (the latter two being the natural degradation products). The modified nucleobases disclosed in Chiu and Rana (2003) RNA 9:1034-1048, Limbach et al. (1994) Nucleic Acids Res. 22:2183-2196 and Revankar and Rao, Comprehensive Natural Products Chemistry, vol. 7, 313, are also contemplated, the contents of which are incorporated herein by reference.

Further examples of base pairing moieties include, but are not limited to, expanded-size nucleobases in which one or more benzene rings has been added. Nucleic base replacements described in the Glen Research catalog (www.glenresearch.com); Krueger AT et al. (2007) Acc. Chem. Res. 40:141-150; Kool E T (2002) Acc. Chem. Res. 35:936-943; Benner S A et al. (2005) Nat. Rev. Genet. 6:553-543; Romesberg F E et al. (2003) Curr. Opin. Chem. Biol. 7:723-733; Hirao, I (2006) Curr. Opin. Chem. Biol. 10:622-627, the contents of which are incorporated herein by reference, are contemplated as useful for the synthesis of the oligomers described herein. Examples of expanded-size nucleobases are shown below:

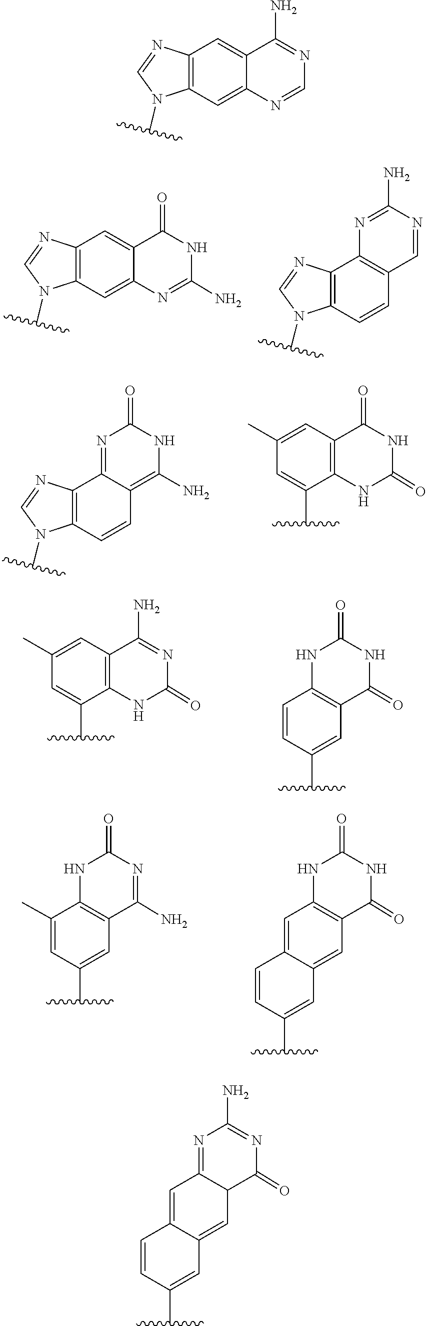

The terms “oligonucleotide” or “oligomer” refer to a compound comprising a plurality of linked nucleosides, nucleotides, or a combination of both nucleosides and nucleotides. In specific embodiments provided herein, an oligonucleotide is a morpholino oligonucleotide.

As used herein, the terms “antisense oligomer” or “antisense compound” are used interchangeably and refer to a sequence of subunits, each having a base carried on a backbone subunit composed of ribose or other pentose sugar or morpholino group, and where the backbone groups are linked by intersubunit linkages that allow the bases in the compound to hybridize to a target sequence in a nucleic acid (typically an RNA) by Watson-Crick base pairing, to form a nucleic acid:oligomer heteroduplex within the target sequence. The oligomer may have exact sequence complementarity to the target sequence or nearly exact complementarity. Such antisense oligomers are designed to block or inhibit translation of the mRNA containing the target sequence, and may be said to be “directed to” a sequence with which it hybridizes.

Also contemplated herein as types of “antisense oligomer” or “antisense compound” are phosphorothioate-modified oligomers, peptide nucleic acids (PNAs), locked nucleic acids (LNAs), 2′-fluoro-modified oligomers, 2′-0,4′-C-ethylene-bridged nucleic acids (ENAs), tricyclo-DNAs, tricylo-DNA phosphorothioate-modified oligomers, 2′-O-[2-(N-methylcarbamoyl) ethyl] modified oligomers, 2′-O-methyl phosphorothioate modified oligomers, 2′-O-methoxyethyl (2′-O-MOE) modified oligomers, and 2′-O-Methyl oligonucleotides, or combinations thereof, as well as other antisense agents known in the art.

An antisense oligomer “specifically hybridizes” to a target polynucleotide if the oligomer hybridizes to the target under physiological conditions, with a Tm greater than 37° C., greater than 45° C., preferably at least 50° C., and typically 60° C.-80° C. or higher. The “Tm” of an oligomer is the temperature at which 50% hybridizes to a complementary polynucleotide. Tm is determined under standard conditions in physiological saline, as described, for example, in Miyada et al. (1987) Methods Enzymol. 154:94-107. Such hybridization may occur with “near” or “substantial” complementarity of the antisense oligomer to the target sequence, as well as with exact complementarity.

The terms “complementary” and “complementarity” refer to oligonucleotides (i.e., a sequence of nucleotides) related by base-pairing rules. For example, the sequence “T-G-A (5-3′)” is complementary to the sequence “T-C-A (5-3′).” Complementarity may be “partial,” in which only some of the nucleic acids' bases are matched according to base pairing rules. Or, there may be “complete,” “total,” or “perfect” (100%) complementarity between the nucleic acids. The degree of complementarity between nucleic acid strands has significant effects on the efficiency and strength of hybridization between nucleic acid strands. While perfect complementarity is often desired, some embodiments can include one or more but preferably 6, 5, 4, 3, 2, or 1 mismatches with respect to the target RNA. Such hybridization may occur with “near” or “substantial” complementarity of the antisense oligomer to the target sequence, as well as with exact complementarity. In some embodiments, an oligomer may hybridize to a target sequence at about 50%, 55%, 60%, 65%, 70%, 75%, 80%, 85%, 90%, 95%, 99% or 100% complementarity. Variations at any location within the oligomer are included. In certain embodiments, variations in sequence near the termini of an oligomer are generally preferable to variations in the interior, and if present are typically within about 6, 5, 4, 3, 2, or 1 nucleotides of the 5-terminus, 3′-terminus, or both termini.

The terms “TEG,” “EG3,” or “triethylene glycol tail” refer to triethylene glycol moieties conjugated to the oligomer, e.g., at its 3′- or 5′-end. For example, in some embodiments, “TEG” includes, for example, wherein A′ of the conjugate of Formula I or Formula IV is of the formula:

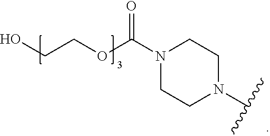

Naturally occurring nucleotide bases include adenine, guanine, cytosine, thymine, and uracil, which have the symbols A, G, C, T, and U, respectively. Nucleotide bases can also encompass analogs of naturally occurring nucleotide bases. Base pairing typically occurs between purine A and pyrimidine T or U, and between purine G and pyrimidine C.

Oligonucleotides may also include nucleobase (often referred to in the art simply as “base”) modifications or substitutions. Oligonucleotides containing a modified or substituted base include oligonucleotides in which one or more purine or pyrimidine bases most commonly found in nucleic acids are replaced with less common or non-natural bases. In some embodiments, the nucleobase is covalently linked at the N9 atom of the purine base, or at the N1 atom of the pyrimidine base, to the morpholine ring of a nucleotide or nucleoside.

Purine bases comprise a pyrimidine ring fused to an imidazole ring, as described by the general formula:

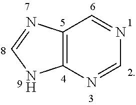

Adenine and guanine are the two purine nucleobases most commonly found in nucleic acids. These may be substituted with other naturally occurring purines, including but not limited to N6-methyladenine, N2-methylguanine, hypoxanthine, and 7-methylguanine.

Pyrimidine bases comprise a six-membered pyrimidine ring as described by the general formula:

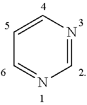

Cytosine, uracil, and thymine are the pyrimidine bases most commonly found in nucleic acids. These may be substituted with other naturally occurring pyrimidines, including but not limited to 5-methylcytosine, 5-hydroxymethylcytosine, pseudouracil, and 4-thiouracil. In one embodiment, the oligonucleotides described herein contain thymine bases in place of uracil.

Other modified or substituted bases include, but are not limited to, 2,6-diaminopurine, orotic acid, agmatidine, lysidine, 2-thiopyrimidine (e.g. 2-thiouracil, 2-thiothymine), G-clamp and its derivatives, 5-substituted pyrimidine (e.g. 5-halouracil, 5-propynyluracil, 5-propynylcytosine, 5-aminomethyluracil, 5-hydroxymethyluracil, 5-aminomethylcytosine, 5-hydroxymethylcytosine, Super T), 7-deazaguanine, 7-deazaadenine, 7-aza-2,6-diaminopurine, 8-aza-7-deazaguanine, 8-aza-7-deazaadenine, 8-aza-7-deaza-2,6-diaminopurine, Super G, Super A, and N4-ethylcytosine, or derivatives thereof; N2-cyclopentylguanine (cPent-G), N2-cyclopentyl-2-aminopurine (cPent-AP), and N2-propyl-2-aminopurine (Pr-AP), pseudouracil or derivatives thereof; and degenerate or universal bases, like 2,6-difluorotoluene or absent bases like a basic sites (e.g. 1-deoxyribose, 1,2-dideoxyribose, I-deoxy-2-O-methylribose; or pyrrolidine derivatives in which the ring oxygen has been replaced with nitrogen (azaribose)). Pseudouracil is a naturally occurring isomerized version of uracil, with a C-glycoside rather than the regular N-glycoside as in uridine.

Certain modified or substituted nucleobases are particularly useful for increasing the binding affinity of the antisense oligonucleotides of the disclosure. These include 5-substituted pyrimidines, 6-azapyrimidines and N-2, N-6, and 0-6 substituted purines, including 2-aminopropyladenine, 5-propynyluracil and 5-propynylcytosine. In various embodiments, nucleobases may include 5-methylcytosine substitutions, which have been shown to increase nucleic acid duplex stability by 0.6-1.2° C.

In some embodiments, modified or substituted nucleobases are useful for facilitating the purification of antisense oligonucleotides. For example, in certain embodiments, antisense oligonucleotides may contain three or more (e.g., 3, 4, 5, 6, or more) consecutive guanine bases. In certain antisense oligonucleotides, a string of three or more consecutive guanine bases can result in aggregation of the oligonucleotides, complicating purification. In such antisense oligonucleotides, one or more of the consecutive guanines can be substituted with hypoxanthine. The substitution of hypoxanthine for one or more guanines in a string of three or more consecutive guanine bases can reduce aggregation of the antisense oligonucleotide, thereby facilitating purification.

The term “abasic subunit” refers to a purine and pyrimidine-free subunit in an antisense oligomer. In an embodiment, an “abasic subuinit” is hydrogen. The abasic subunits incorporated herein retain the antisense backbone but do not contain purine or pyrimidine bases. A non-limiting example of an antisense oligomer comprising an abasic subunit is depicted below.

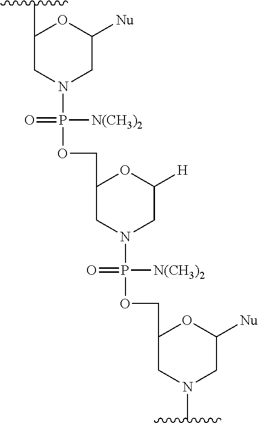

The oligonucleotides provided herein are synthesized and do not include antisense compositions of biological origin. The molecules of the disclosure may also be mixed, encapsulated, conjugated, or otherwise associated with other molecules, molecule structures, or mixtures of compounds, as for example, liposomes, receptor-targeted molecules, oral, rectal, topical, or other formulations, for assisting in uptake, distribution, or absorption, or a combination thereof.

As used herein, a “nucleic acid analog” refers to a non-naturally occurring nucleic acid molecule. A nucleic acid is a polymer of nucleotide subunits linked together into a linear structure. Each nucleotide consists of a nitrogen-containing aromatic base attached to a pentose (five-carbon) sugar, which is in turn attached to a phosphate group. Successive phosphate groups are linked together through phosphodiester bonds to form the polymer. The two common forms of naturally occurring nucleic acids are deoxyribonucleic acid (DNA) and ribonucleic acid (RNA). One end of the chain carries a free phosphate group attached to the 5-carbon atom of a sugar moiety; this is called the 5′ end of the molecule. The other end has a free hydroxyl (—OH) group at the 3′-carbon of a sugar moiety and is called the 3′ end of the molecule. A nucleic acid analog can include one or more non-naturally occurring nucleobases, sugars, and/or internucleotide linkages, for example, a phosphorodiamidate morpholino oligomer (PMO). As disclosed herein, in certain embodiments, a “nucleic acid analog” is a PMO, and in certain embodiments, a “nucleic acid analog” is a positively charged cationic PMO.

A “morpholino oligomer” or “PMO” refers to a polymeric molecule having a backbone that supports bases capable of hydrogen bonding to typical polynucleotides, wherein the polymer lacks a pentose sugar backbone moiety, and more specifically a ribose backbone linked by phosphodiester bonds which is typical of nucleotides and nucleosides, but instead contains a ring nitrogen with coupling through the ring nitrogen. An exemplary “morpholino” oligomer comprises morpholino subunit structures linked together by phosphoramidate or phosphorodiamidate linkages, joining the morpholino nitrogen of one subunit to the 5′ exocyclic carbon of an adjacent subunit, each subunit comprising a purine or pyrimidine base-pairing moiety effective to bind, by base-specific hydrogen bonding, to a base in a polynucleotide. Morpholino oligomers (including antisense oligomers) are detailed, for example, in U.S. Pat. Nos. 5,034,506; 5,142,047; 5,166,315; 5,185,444; 5,217,866; 5,506,337; 5,521,063; 5,698,685; 8,076,476; and 8,299,206; and PCT publication number WO 2009/064471, all of which are incorporated herein by reference in their entirety.

A preferred morpholino oligomer is a phosphorodiamidate-linked morpholino oligomer, referred to herein as a PMO. Such oligomers are composed of morpholino subunit structures such as those shown below:

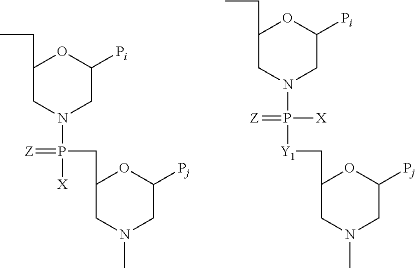

where X is NH₂, NHR, or NR₂ (where R is lower alkyl, preferably methyl), Y₁ is O, and Z is O, and Pᵢ and Pⱼ are purine or pyrimidine base-pairing moieties effective to bind, by base-specific hydrogen bonding, to a base in a polynucleotide. Also preferred are structures having an alternate phosphorodiamidate linkage, where X is lower alkoxy, such as methoxy or ethoxy, Y₁ is NH or NR, where R is lower alkyl, and Z is O.

Representative PMOs include PMOs wherein the intersubunit linkages are linkage (A1). See Table 1.

TABLE 1
Representative Intersubunit Linkages

| No. | Name | Structure |
|---|---|---|
| A1 | PMO | 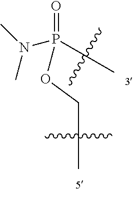 [SMILES: *COP(*)(=O)N(C)C] |
| A2 | PMO⁺ (unprotonated from depicted) | 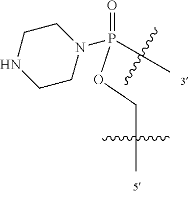 [SMILES: *COP(*)(=O)N1CCNCC1] |
| A3 | PMO⁺ (+) | 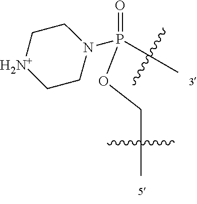 [SMILES: *COP(*)(=O)N1CC[NH2+]CC1] |

A phosphoramidate group comprises phosphorus having three attached oxygen atoms and one attached nitrogen atom, while a “phosphorodiamidate” group comprises phosphorus having two attached oxygen atoms and two attached nitrogen atoms. A representative phosphorodiamidate example is below:

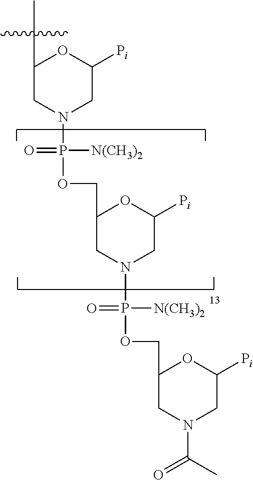

each Pᵢ is independently selected from H, a nucleobase, and a nucleobase functionalized with a chemical protecting-group, wherein the nucleobase independently at each occurrence comprises a C₃₋₆ heterocyclic ring selected from pyridine, pyrimidine, triazinane, purine, and deaza-purine; and n is an integer of 6-38. The ring nitrogen of the subunit at the 3′ terminus of a PMO may be capped with a capping group such as acetyl or may be uncapped with a free hydrogen.

In the uncharged or the modified intersubunit linkages of the oligomers described herein, one nitrogen is always pendant to the backbone chain. The second nitrogen, in a phosphorodiamidate linkage, is typically the ring nitrogen in a morpholino ring structure.

PMOs are water-soluble, uncharged, or substantially uncharged antisense molecules that inhibit gene expression by preventing binding or progression of splicing or translational machinery components. PMOs have also been shown to inhibit or block viral replication (Stein, Skilling et al. 2001; McCaffrey, Meuse et al. 2003). They are highly resistant to enzymatic digestion (Hudziak, Barofsky et al. 1996). PMOs have demonstrated high antisense specificity and efficacy in vitro in cell-free and cell culture models (Stein, Foster et al. 1997; Summerton and Weller 1997), and in vivo in zebrafish, frog, and sea urchin embryos (Heasman, Kofron et al. 2000; Nasevicius and Ekker 2000), as well as in adult animal models, such as rats, mice, rabbits, dogs, and pigs (see e.g. Arora and Iversen 2000; Qin, Taylor et al. 2000; Iversen 2001; Kipshidze, Keane et al. 2001; Devi 2002; Devi, Oldenkamp et al. 2002; Kipshidze, Kim et al. 2002; Ricker, Mata et al. 2002).

Antisense PMO oligomers have been shown to be taken up into cells and to be more consistently effective in vivo, with fewer nonspecific effects, than other widely used antisense oligonucleotides (see e.g. P. Iversen, “Phosphoramidite Morpholino Oligomers,” in Antisense Drug Technology, S. T. Crooke, ed., Marcel Dekker, Inc., New York, 2001). Conjugation of PMOs to arginine-rich peptides has been shown to increase their cellular uptake (see e.g., U.S. Pat. No. 7,468,418, incorporated herein by reference in its entirety).

“Charged,” “uncharged,” “cationic,” and “anionic” as used herein refer to the predominant state of a chemical moiety at near-neutral pH, e.g., about 6 to 8. For example, the term may refer to the predominant state of the chemical moiety at physiological pH, that is, about 7.4.

A “cationic PMO” or “PMO+” refers to a phosphorodiamidate morpholino oligomer comprising any number of (1-piperazino)phosphinylideneoxy, (1-(4-(ω-guanidino-alkanoyl))-piperazino)phosphinylideneoxy linkages (A2 and A3; see Table 1) that have been described previously (see e.g., PCT publication WO 2008/036127 which is incorporated herein by reference in its entirety).

The “backbone” of an oligonucleotide analog (e.g., an uncharged oligonucleotide analogue) refers to the structure supporting the base-pairing moieties; e.g., for a morpholino oligomer, as described herein, the “backbone” includes morpholino ring structures connected by intersubunit linkages (e.g., phosphorus-containing linkages). A “substantially uncharged backbone” refers to the backbone of an oligonucleotide analogue wherein less than 50% of the intersubunit linkages are charged at near-neutral pH. For example, a substantially uncharged backbone may comprise less than 50%, less than 40%, less than 30%, less than 20%, less than 10%, less than 5% or even 0% intersubunit linkages which are charged at near neutral pH. In some embodiments, the substantially uncharged backbone comprises at most one charged (at physiological pH) intersubunit linkage for every four uncharged (at physiological pH) linkages, at most one for every eight or at most one for every sixteen uncharged linkages. In some embodiments, the nucleic acid analogs described herein are fully uncharged.

The term “targeting base sequence” or simply “targeting sequence” is the sequence in the nucleic acid analog that is complementary (meaning, in addition, substantially complementary) to a target sequence, e.g., a target sequence in the RNA genome of humans. The entire sequence, or only a portion, of the analog compound may be complementary to the target sequence. For example, in an analog having 20 bases, only 12-14 may be targeting sequences. Typically, the targeting sequence is formed of contiguous bases in the analog, but may alternatively be formed of non-contiguous sequences that when placed together, e.g., from opposite ends of the analog, constitute a sequence that spans the target sequence.

The term “peptide” refers to a compound comprising a plurality of linked amino acids. The peptides provided herein can be considered to be cell-penetrating peptides.

As used herein, a “cell-penetrating peptide” (CPP) or “carrier peptide” is a relatively short peptide capable of promoting the uptake of PMOs by cells, thereby delivering the PMOs to the interior (cytoplasm) of the cells. The CPP or carrier peptide typically is about 12 to about 40 amino acids long. The length of the carrier peptide is not particularly limited and varies in different embodiments. In some embodiments, the carrier peptide comprises from 4 to 40 amino acid subunits. In other embodiments, the carrier peptide comprises from 6 to 30, from 6 to 20, from 8 to 25, or from 10 to 20 amino acid subunits. In various embodiments, a CPP embodiment of the disclosure may include an arginine-rich peptide as described further below.

As used herein, a “peptide-conjugated phosphorodiamidate-linked morpholino oligomer” or “PPMO” refers to a PMO covalently linked to a peptide, such as a cell-penetrating peptide (CPP) or carrier peptide. The cell-penetrating peptide promotes the uptake of the PMO by cells, thereby delivering the PMO to the interior (cytoplasm) of the cells. Depending on its amino acid sequence, a CPP can be generally effective or it can be specifically or selectively effective for PMO delivery to a particular type or particular types of cells. PMOs and CPPs are typically linked at their ends, e.g., the C-terminal end of the CPP can be linked to the 5′ end of the PMO, or the 3′ end of the PMO can be linked to the N-terminal end of the CPP. PPMOs can include uncharged PMOs, charged (e.g., cationic) PMOs, and mixtures thereof. In an embodiment, the linking moiety of the conjugates described herein may be cleaved to release a PPMO.

The carrier peptide may be linked to the nucleic acid analog either directly or via an optional linker, e.g., one or more additional naturally occurring amino acids, e.g., cysteine (C), glycine (G), or proline (P), or additional amino acid analogs, e.g., 6-aminohexanoic acid (X), beta-alanine (B), or XB. Other linking moieties known in the art may also be employed.

An “amino acid subunit” is generally an α-amino acid residue (—CO—CHR—NH—); but may also be a β- or other amino acid residue (e.g., —CO—CH₂CHR—NH—), where R is an amino acid side chain.

The term “naturally occurring amino acid” refers to an amino acid present in proteins found in nature; examples include Alanine (A), Cysteine (C), Aspartic acid (D), Glutamic acid (E), Phenyalanine (F), Glycine (G), Histidine (H), Isoleucine (I), Lysine (K), Leucine (L). Methionine (M), Asparagine (N), Proline (P), Glutamine (Q), Arginine (R), Serine (S), Threonine (T), Valine (V), Tryptophan (W), and Tyrosine (Y). The term “non-natural amino acids” refers to those amino acids not present in proteins found in nature; examples include beta-alanine (β-Ala) and 6-aminohexanoic acid (Ahx).

An agent is “actively taken up by mammalian cells” when the agent can enter the cell by a mechanism other than passive diffusion across the cell membrane. The agent may be transported, for example, by “active transport,” referring to transport of agents across a mammalian cell membrane by, e.g., an ATP-dependent transport mechanism, or by “facilitated transport,” referring to transport of antisense agents across the cell membrane by a transport mechanism that requires binding of the agent to a transport protein, which then facilitates passage of the bound agent across the membrane.

As used herein, an “effective amount” refers to any amount of a substance that is sufficient to achieve a desired biological result. A “therapeutically effective amount” refers to any amount of a substance that is sufficient to achieve a desired therapeutic result.

As used herein, a “subject” is a mammal, which can include a mouse, rat, hamster, guinea pig, rabbit, goat, sheep, cat, dog, pig, cow, horse, monkey, non-human primate, or human. In certain embodiments, a subject is a human.

“Treatment” of an individual (e.g., a mammal, such as a human) or a cell is any type of intervention used to alter the natural course of the individual or cell. Treatment includes, but is not limited to, administration of a pharmaceutical composition, and may be performed either prophylactically or subsequent to the initiation of a pathologic event or contact with an etiologic agent.

### II. Peptide-Oligonucleotides

In some embodiments provided herein, is an antisense oligomer comprising a modified antisense oligonucleotide, wherein:
the modified antisense oligonucleotide is 18-40 subunits in length, comprising a targeting sequence complementary to a target region within intron 1 (SEQ ID NO: 1) of a pre-mRNA of human acid alpha-glucosidase (GAA) gene, wherein:
the antisense oligonucleotide comprises a morpholino oligomer;
each subunit of the antisense oligonucleotide comprises a nucleobase or is an abasic subunit, wherein each subunit is taken together in order from the 5′ end of the antisense oligonucleotide to the 3′ end of the antisense oligonucleotide form the targeting sequence;
at least one subunit is an abasic subunit; and
wherein the targeting sequence, except for the abasic subunit or subunits, is at least 80% complementary to the target region.

In an embodiment, the abasic subunit is internal to the targeting sequence.

In an embodiment, the modified antisense oligonucleotide is 20-40 subunits in length. In another embodiment, the modified antisense oligonucleotide is 19-29 subunits in length. In another embodiment, the modified antisense oligonucleotide is 18-40, 19-30, 19-29, 20-40, 20-30, 20-25, 21-40, 21-30, 21-25, 22-40, 22-30, 22-25, 23-40, 23-30, or 23-25 subunits in length. In still another embodiment, the modified antisense oligonucleotide is 19, 20, 21, 22, 23, 24, 25, 26, 27, 28, 29, 30, 31, 32, 33, 34, 35, 36, 37, 38, 39, or 40 subunits in length.

In an embodiment, the modified antisense oligonucleotide is an antisense oligomer of Formula I:

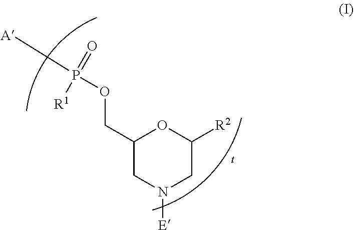

or a pharmaceutically acceptable salt thereof,
wherein:
A′ is selected from —N(H)CH₂C(O)NH₂, —N(C₁₋₆-alkyl)CH₂C(O)NH₂,

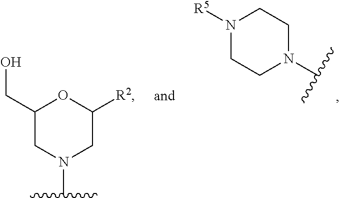

wherein
R⁵ is —C(O)(O-alkyl)ₓ-OH, wherein x is 3-10 and each alkyl group is, independently at each occurrence, C₂₋₆-alkyl,
or R⁵ is selected from H, —C(O)C₁₋₆-alkyl, trityl, monomethoxytrityl, —(C₁₋₆-alkyl)-R⁶, —(C₁₋₆-heteroalkyl)-R⁶, aryl-R⁶, heteroaryl-R⁶, —C(O)O—(C₁₋₆-alkyl)-R⁶, —C(O)O-aryl-R⁶, —C(O)O-heteroaryl-R⁶, and

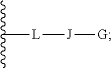

R⁶ is selected from OH, SH, and NH₂, or R⁶ is O, S, or NH, each of which is covalently linked to a solid support;
each R¹ is independently selected from OH and —N(R³)(R⁴), wherein each R³ and R⁴ are, independently at each occurrence, H or —C₁₋₆-alkyl;
each R² is independently, at each occurrence, selected from H (abasic), a nucleobase, and a nucleobase functionalized with a chemical protecting group, wherein the nucleobase, independently at each occurrence, comprises a C₃₋₆-heterocyclic ring selected from pyridine, pyrimidine, purine, and deaza-purine;
t is 8-40;
E′ is selected from H, —C₁₋₆-alkyl, —C(O)C₁₋₆-alkyl, benzoyl, stearoyl, trityl, monomethoxytrityl, dimethoxytrityl, trimethoxytrityl,

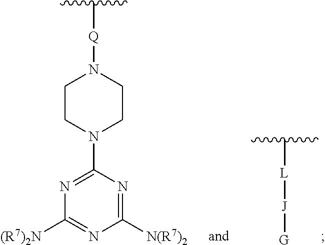

wherein
   Q is —C(O)(CH₂)₆C(O)— or —C(O)(CH₂)₂S₂(CH₂)₂C(O)—;
R⁷ is —(CH₂)₂OC(O)N(R⁸)₂, wherein R⁸ is —(CH₂)₆NHC(═NH)NH₂;
L is selected from glycine, proline, W, W—W, or R⁹, wherein L is covalently linked by an amide bond to the N-terminus or C-terminus of J;
W is —C(O)—(CH₂)ₘ—NH—, wherein m is 2 to 12;
R⁹ is selected from the group consisting of:

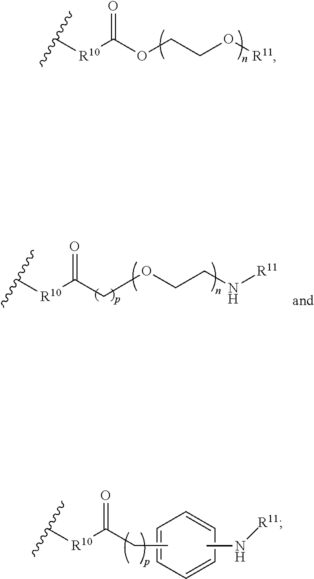

n is 1, 2, 3, 4, 5, 6, 7, 8, 9, or 10;
p is 2, 3, 4, or 5;
R¹⁰ is selected from a bond, glycine, proline, W, or W—W;
R¹¹ is selected from the group consisting of glycine, proline, W, W—W, and

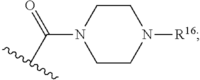

R¹⁶ is selected from a bond, glycine, proline, W, or W—W; wherein R¹⁶ is covalently linked by an amide bond to the N-terminus or C-terminus of J; J is a cell-penetrating peptide; and
G is selected from H, —C(O)C₁₋₆-alkyl, benzoyl, and stearoyl, wherein G is covalently linked to J.

In an aspect, disclosed herein is an antisense oligomer, wherein the antisense oligomer is a conjugate comprising a modified antisense oligonucleotide and a cell-penetrating peptide, wherein:
the modified antisense oligonucleotide is 18-40 subunits in length, comprising a targeting sequence complementary to a target region within intron 1 (SEQ ID NO: 1) of a pre-mRNA of human acid alpha-glucosidase (GAA) gene, wherein
the antisense oligonucleotide comprises a morpholino oligomer;
the antisense oligonucleotide is covalently linked to the cell-penetrating peptide;
each subunit of the antisense oligonucleotide comprises a nucleobase or is an abasic subunit, wherein each subunit is taken together in order from the 5′ end of the antisense oligonucleotide to the 3′ end of the antisense oligonucleotide form the targeting sequence;
at least one subunit is an abasic subunit; and
wherein the targeting sequence, except for the abasic subunit or subunits, is at least 80% complementary to the target region.

In an embodiment, the modified antisense oligonucleotide is 20-40 subunits in length. In another embodiment, the modified antisense oligonucleotide is 19-29 subunits in length.

In an embodiment, the target region comprises a sequence selected from the group consisting of SEQ ID NO: 2 (GAA-IVS1(-189-167)) and SEQ ID NO: 3 (GAA-IVS1(-80-24)). In a further embodiment, the target region comprises the sequence set forth as SEQ ID NO: 2. In another embodiment, the target region comprises the sequence set forth as SEQ ID NO: 3.

In an embodiment, target region is selected from GAA-IVS1(-189-167), GAA-IVS1(-80-56), GAA-IVS1(-76-52), GAA-IVS1(-74-55), GAA-IVS1(-72-48), GAA-IVS1(-71-47), GAA-IVS1(-70-46), GAA-IVS1(-69-45), GAA-IVS1(-66-42), GAA-IVS1(-65-41), and GAA-IVS1(-49-24). In a further embodiment, the target region is GAA-IVS1(-189-167). In another embodiment, the targeting region is GAA-IVS1(-72,-48). In still another embodiment, the targeting region is GAA-IVS1(-71,-47). In yet another embodiment, the targeting region is GAA-IVS1(-70,-46). In an embodiment, the targeting region is GAA-IVS1(-69-45). In another embodiment, the targeting region is GAA-IVS1(-65,-41). In still another embodiment, the targeting region is GAA-IVS1(-66,-42).

In an embodiment, the targeting sequence comprises the sequence CCA GAA GGA AXX XCG AGA AAA GC (SEQ ID NO: 4), wherein each X is independently selected from guanine (G) or is abasic (B), wherein at least one X is B. In another embodiment, the targeting sequence comprises a sequence selected from the group consisting of:

i)
SEQ ID NO: 5
(CCA GAA GGA AGG BCG AGA AAA GC);
ii)
SEQ ID NO: 6
(CCA GAA GGA AGB GCG AGA AAA GC);
iii)
SEQ ID NO: 7
(CCA GAA GGA ABG GCG AGA AAA GC);
iv)
SEQ ID NO: 8
(CCA GAA GGA AGB BCG AGA AAA GC);
v)
SEQ ID NO: 9
(CCA GAA GGA ABB GCG AGA AAA GC);
and
vi)
SEQ ID NO: 10
(CCA GAA GGA ABG BCG AGA AAA GC).

In an embodiment, B is H.

In an embodiment, the targeting sequence comprises SEQ ID NO: 5 (CCA GAA GGA AGG BCG AGA AAA GC). In another embodiment, the targeting sequence comprises SEQ ID NO: 6 (CCA GAA GGA AGB GCG AGA AAA GC). In still another embodiment, the targeting sequence comprises SEQ ID NO: 7 (CCA GAA GGA ABG GCG AGA AAA GC). In yet another embodiment, the targeting sequence comprises SEQ ID NO: 8 (CCA GAA GGA AGB BCG AGA AAA GC). In an embodiment, the targeting sequence comprises SEQ ID NO: 9 (CCA GAA GGA ABB GCG AGA AAA GC). In another embodiment, the targeting sequence comprises SEQ ID NO: 10 (CCA GAA GGA ABG BCG AGA AAA GC).

In an embodiment, the targeting sequence consists of the sequence CCA GAA GGA AXX XCG AGA AAA GC (SEQ ID NO: 4). In another embodiment, the targeting sequence consists of SEQ ID NO: 5 (CCA GAA GGA AGG BCG AGA AAA GC). In still another embodiment, the targeting sequence consists of SEQ ID NO: 6 (CCA GAA GGA AGB GCG AGA AAA GC). In yet another embodiment, the targeting sequence consists of SEQ ID NO: 7 (CCA GAA GGA ABG GCG AGA AAA GC). In an embodiment, the targeting sequence consists of SEQ ID NO: 8 (CCA GAA GGA AGB BCG AGA AAA GC). In another embodiment, the targeting sequence consists of SEQ ID NO: 9 (CCA GAA GGA ABB GCG AGA AAA GC). In still another embodiment, the targeting sequence consists of SEQ ID NO: 10 (CCA GAA GGA ABG BCG AGA AAA GC).

In an embodiment, the target region is selected from the group consisting of GAA-IVS1(-80-56), GAA-IVS1(-76-52), GAA-IVS1(-74-55), GAA-IVS1(-72-48), GAA-IVS1(-71-47), GAA-IVS1(-70-46), GAA-IVS1(-69-45), GAA-IVS1(-66-42), GAA-IVS1(-65-41), and GAA-IVS1(-49-24). In another embodiment, the target region is selected from the group consisting of GAA-IVS1(-72-48), GAA-IVS1(-71-47), GAA-IVS1(-70-46), GAA-IVS1(-69-45), GAA-IVS1(-66-42), and GAA-IVS1(-65-41).

In an embodiment, the targeting sequence comprises a sequence selected from the group consisting of:

i)
SEQ ID NO: 11
(CTC ACX XXX CTC TCA AAG CAG CTC T);
ii)
SEQ ID NO: 12
(ACT CAC XXX XCT CTC AAA GCA GCT C);
iii)
SEQ ID NO: 13
(CAC TCA CXX XXC TCT CAA AGC AGC T);
iv)
SEQ ID NO: 14
(GCA CTC ACX XXX CTC TCA AAG CAG C);
v)
SEQ ID NO: 15
(GCG GCA CTC ACX XXX CTC TCA AAG C);
vi)
SEQ ID NO: 16
(GGC GGC ACT CAC XXX XCT CTC AAA G);

wherein each X is independently selected from guanine (G) or is abasic (B), wherein at least one X is B. In an embodiment, the targeting sequence is selected from the group consisting of:

i)
SEQ ID NO: 17
(GCA CTC ACB GGG CTC TCA AAG CAG C);
ii)
SEQ ID NO: 18
(GCA CTC ACG BGG CTC TCA AAG CAG C);
iii)
SEQ ID NO: 19
(GCA CTC ACG GBG CTC TCA AAG CAG C);
iv)
SEQ ID NO: 20
(GCA CTC ACG GGB CTC TCA AAG CAG C);
v)
SEQ ID NO: 21
(GCA CTC ACB BGG CTC TCA AAG CAG C);
vi)
SEQ ID NO: 22
(GCA CTC ACG BBG CTC TCA AAG CAG C);
vii)
SEQ ID NO: 23
(GCA CTC ACG GBB CTC TCA AAG CAG C);
and
viii)
SEQ ID NO: 24
(GGC GGC ACT CAC GBB GCT CTC AAA G).

In an embodiment, B is H.

In an embodiment, the targeting sequence comprises SEQ ID NO: 11 (CTC ACX XXX CTC TCA AAG CAG CTC T). In another embodiment, the targeting sequence comprises SEQ ID NO: 12 (ACT CAC XXX XCT CTC AAA GCA GCT C). In still another embodiment, the targeting sequence comprises SEQ ID NO: 13 (CAC TCA CXX XXC TCT CAA AGC AGC T). In yet another embodiment, the targeting sequence comprises SEQ ID NO: 14 (GCA CTC ACX XXX CTC TCA AAG CAG C). In an embodiment, the targeting sequence comprises SEQ ID NO: 15 (GCG GCA CTC ACX XXX CTC TCA AAG C). In another embodiment, the targeting sequence comprises SEQ ID NO: 16 (GGC GGC ACT CAC XXX XCT CTC AAA G). In still another embodiment, the targeting sequence comprises SEQ ID NO: 17 (GCA CTC ACB GGG CTC TCA AAG CAG C). In yet another embodiment, the targeting sequence comprises SEQ ID NO: 18 (GCA CTC ACG BGG CTC TCA AAG CAG C). In an embodiment, the targeting sequence comprises SEQ ID NO: 19 (GCA CTC ACG GBG CTC TCA AAG CAG C). In another embodiment, the targeting sequence comprises SEQ ID NO: 20 (GCA CTC ACG GGB CTC TCA AAG CAG C). In still another embodiment, the targeting sequence comprises SEQ ID NO: 21 (GCA CTC ACB BGG CTC TCA AAG CAG C). In yet another embodiment, the targeting sequence comprises SEQ ID NO: 22 (GCA CTC ACG BBG CTC TCA AAG CAG C). In an embodiment, the targeting sequence comprises SEQ ID NO: 23 (GCA CTC ACG GBB CTC TCA AAG CAG C). In another embodiment, the targeting sequence comprises SEQ ID NO: 24 (GGC GGC ACT CAC GBB GCT CTC AAA G).

In an embodiment, the targeting sequence consists of SEQ ID NO: 11 (CTC ACX XXX CTC TCA AAG CAG CTC T). In another embodiment, the targeting sequence consists of SEQ ID NO: 12 (ACT CAC XXX XCT CTC AAA GCA GCT C). In still another embodiment, the targeting sequence consists of SEQ ID NO: 13 (CAC TCA CXX XXC TCT CAA AGC AGC T). In yet another embodiment, the targeting sequence consists of SEQ ID NO: 14 (GCA CTC ACX XXX CTC TCA AAG CAG C). In an embodiment, the targeting sequence consists of SEQ ID NO: 15 (GCG GCA CTC ACX XXX CTC TCA AAG C). In another embodiment, the targeting sequence consists of SEQ ID NO: 16 (GGC GGC ACT CAC XXX XCT CTC AAA G). In still another embodiment, the targeting sequence consists of SEQ ID NO: 17 (GCA CTC ACB GGG CTC TCA AAG CAG C). In yet another embodiment, the targeting sequence consists of SEQ ID NO: 18 (GCA CTC ACG BGG CTC TCA AAG CAG C). In an embodiment, the targeting sequence consists of SEQ ID NO: 19 (GCA CTC ACG GBG CTC TCA AAG CAG C). In another embodiment, the targeting sequence consists of SEQ ID NO: 20 (GCA CTC ACG GGB CTC TCA AAG CAG C). In still another embodiment, the targeting sequence consists of SEQ ID NO: 21 (GCA CTC ACB BGG CTC TCA AAG CAG C). In yet another embodiment, the targeting sequence consists of SEQ ID NO: 22 (GCA CTC ACG BBG CTC TCA AAG CAG C). In an embodiment, the targeting sequence consists of SEQ ID NO: 23 (GCA CTC ACG GBB CTC TCA AAG CAG C). In another embodiment, the targeting sequence consists of SEQ ID NO: 24 (GGC GGC ACT CAC GBB GCT CTC AAA G).

In one embodiment, the targeting sequence, except for the abasic subunit or subunits, is at least 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99 percent complementary to the target region. In another embodiment, the targeting sequence, except for the abasic subunit or subunits, is at least 84%, at least 88%, or at least 92% complementary to the target region. In still another embodiment, the targeting sequence, except for the abasic subunit or subunits, is at least 90% complementary to the target region. In yet another embodiment, the targeting sequence, except for the abasic subunit or subunits, is at least 95% complementary to the target region. In still another embodiment, the targeting sequence, except for the abasic subunit or subunits, is 100% complementary to the target region.

In an embodiment, each abasic subunit is at least 8 subunits from the 5′ or 3′ end of the targeting sequence.

The antisense oligonucleotide can comprise 1 to 5 abasic subunits. In an embodiment, the antisense oligonucleotide comprises 1, 2, 3, or 4 abasic subunits.

In another embodiment, the antisense oligomer is an antisense-oligomer-conjugate having the Formula IV:

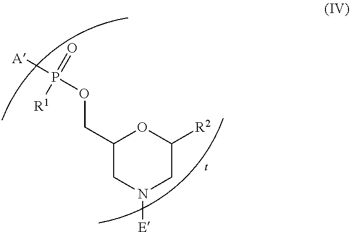

or a pharmaceutically acceptable salt thereof,
wherein:
A′ is selected from —N(H)CH₂C(O)NH₂, —N(C₁₋₆-alkyl)CH₂C(O)NH₂,

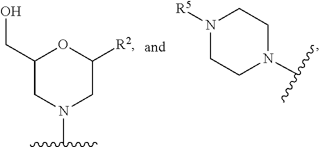

wherein
R⁵ is —C(O)(O-alkyl)ₓ-OH, wherein x is 3-10 and each alkyl group is, independently at each occurrence, C₂₋₆-alkyl,
or R⁵ is selected from H, —C(O)C₁₋₆-alkyl, trityl, monomethoxytrityl, —(C₁₋₆-alkyl)-R⁶, —(C₁₋₆-heteroalkyl)-R⁶, aryl-R⁶, heteroaryl-R⁶, —C(O)O—(C₁₋₆-alkyl)-R⁶, —C(O)O-aryl-R⁶, —C(O)O-heteroaryl-R⁶, and

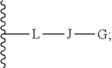

R⁶ is selected from OH, SH, and NH₂, or R⁶ is O, S, or NH, each of which is covalently linked to a solid support;
each R¹ is independently selected from OH and —N(R³)(R⁴), wherein each R³ and R⁴ are, independently at each occurrence, H or —C₁₋₆-alkyl;
each R² is independently, at each occurrence, selected from H (abasic), a nucleobase, and a nucleobase functionalized with a chemical protecting group, wherein the nucleobase, independently at each occurrence, comprises a C₃₋₆-heterocyclic ring selected from pyridine, pyrimidine, purine, and deaza-purine;
t is 8-40;
E′ is selected from H, —C₁₋₆-alkyl, —C(O)C₁₋₆-alkyl, benzoyl, stearoyl, trityl, monomethoxytrityl, dimethoxytrityl, trimethoxytrityl,

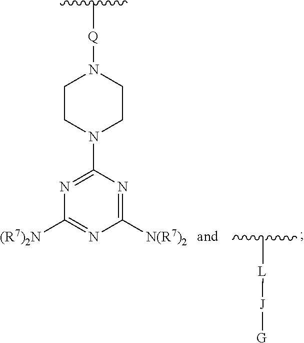

wherein
   Q is —C(O)(CH₂)₆C(O)— or —C(O)(CH₂)₂S₂(CH₂)₂C(O)—;
R⁷ is —(CH₂)₂OC(O)N(R⁸)₂, wherein R⁸ is —(CH₂)₆NHC(═NH)NH₂;
L is selected from glycine, proline, W, W—W, or R⁹, wherein L is covalently linked by an amide bond to the N-terminus or C-terminus of J;
W is —C(O)—(CH₂)ₘ—NH—, wherein m is 2 to 12;
R⁹ is selected from the group consisting of:

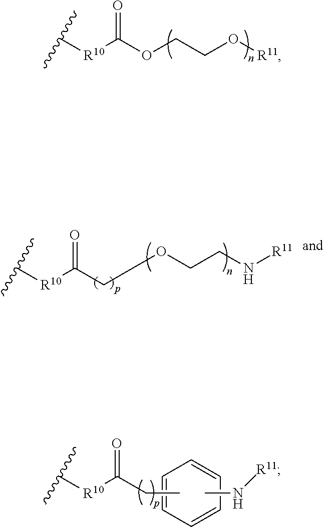

n is 1, 2, 3, 4, 5, 6, 7, 8, 9, or 10;
p is 2, 3, 4, or 5;
R¹⁰ is selected from a bond, glycine, proline, W, or W—W;
R¹¹ is selected from the group consisting of glycine, proline, W, W—W, and

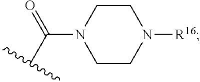

R¹⁶ is selected from a bond, glycine, proline, W, or W—W; wherein R¹⁶ is covalently linked by an amide bond to the N-terminus or C-terminus of J; J is a cell-penetrating peptide; and
G is selected from H, —C(O)C₁₋₆-alkyl, benzoyl, and stearoyl, wherein G is covalently linked to J;
provided that

A′ is

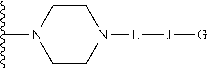

### or E′ is

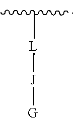

In an embodiment, E′ is selected from H, —C₁₋₆-alkyl, —C(O)C₁₋₆-alkyl, benzoyl, stearoyl, trityl, monomethoxytrityl, dimethoxytrityl, trimethoxytrityl, and

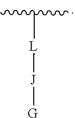

In an embodiment, A′ is selected from —N(C₁₋₆-alkyl)CH₂C(O)NH₂,

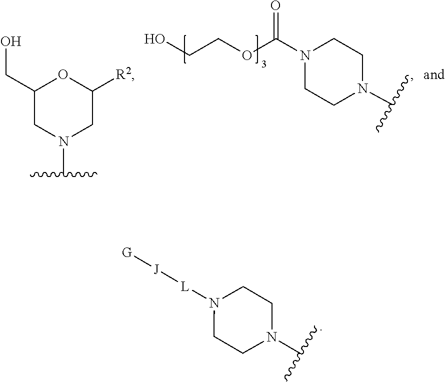

In an embodiment, E′ is selected from H, —C(O)CH₃, benzoyl, stearoyl, trityl, 4-methoxytrityl, and

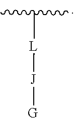

In an embodiment, A′ is selected from —N(C₁₋₆-alkyl)CH₂C(O)NH₂,

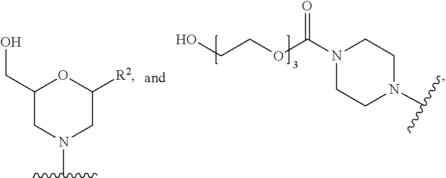

and
E′ is

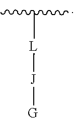

In an embodiment, A′ is

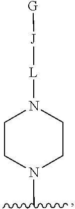

and

E′ is selected from H, —C(O)CH₃, trityl, 4-methoxytrityl, benzoyl, and stearoyl.

In an embodiment, the conjugate of Formula IV is a conjugate selected from:

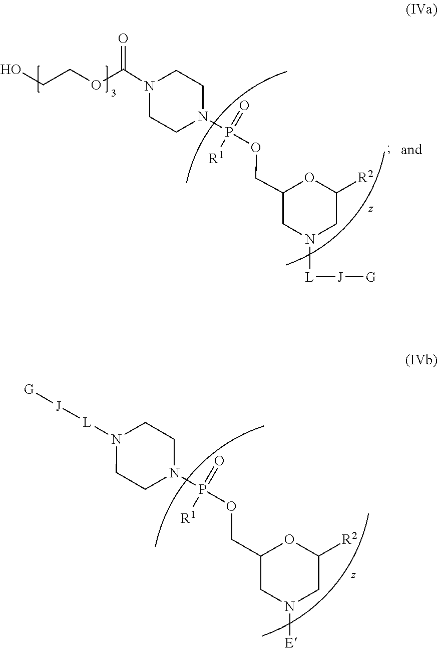

wherein E′ is selected from H, C₁₋₆-alkyl, —C(O)CH₃, benzoyl, and stearoyl.

In an embodiment, the conjugate is of the Formula (IVa). In an embodiment, the conjugate is of the Formula (IVb).

In an embodiment, each R¹ is —N(CH₃)₂.

In an embodiment, each nucleobase, independently at each occurrence, is selected from adenine, guanine, cytosine, 5-methyl-cytosine, thymine, uracil, and hypoxanthine. In an embodiment, L is glycine. In an embodiment, L is proline. In an embodiment, L is —C(O)—(CH₂)₅—NH—. In an embodiment, L is —C(O)—(CH₂)₂—NH—. In an embodiment, L is —C(O)—(CH₂)₂—NH—C(O)—(CH₂)₅—NH—.

In an embodiment, L is

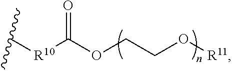

wherein R¹⁰ is a bond, and R¹¹ is selected from: glycine and

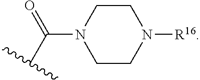

In an embodiment, L is

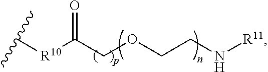

wherein R¹⁰ is a bond; and R¹¹ is selected from: glycine and

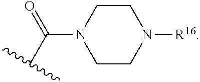

In an embodiment, L is

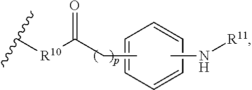

wherein R¹⁰ is a bond; and R¹¹ is selected from: glycine and

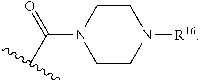

In an embodiment, J is selected from rTAT, TAT, R₉F₂, R₅F₂R₄, R₄, R₅, R₆, R₇, Ra, R₉, (RXR)₄, (RXR)₅, (RXRRBR)₂, (RAR)₄F₂, (RGR)₄F₂.

In an embodiment, G is selected from H, C(O)CH₃, benzoyl, and stearoyl. In an embodiment, G is H or —C(O)CH₃. In an embodiment, G is H. In an embodiment, G is —C(O)CH₃.

In an embodiment, the targeting sequence complementary to a target region within intron 1 (SEQ ID NO: 1) of a pre-mRNA of human acid alpha-glucosidase (GAA) gene, wherein at least one subunit is an abasic subunit. In another embodiment, the target region comprises a sequence selected from the group consisting of SEQ ID NO: 2 (GAA-IVS1(-189-167)) and SEQ ID NO: 3 (GAA-IVS1(-80-24)).

In an embodiment, the targeting sequence comprises the sequences:

i)
SEQ ID NO: 4
(CCA GAA GGA AXX XCG AGA AAA GC);
ii)
SEQ ID NO: 11
(CTC ACX XXX CTC TCA AAG CAG CTC T);
iii)
SEQ ID NO: 12
(ACT CAC XXX XCT CTC AAA GCA GCT C);
iv)
SEQ ID NO: 13
(CAC TCA CXX XXC TCT CAA AGC AGC T);
v)
SEQ ID NO: 14
(GCA CTC ACX XXX CTC TCA AAG CAG C);
vi)
SEQ ID NO: 15
(GCG GCA CTC ACX XXX CTC TCA AAG C);
vii)
SEQ ID NO: 16
(GGC GGC ACT CAC XXX XCT CTC AAA G);

wherein each X is independently selected from guanine (G) or is abasic (B), wherein at least one X is B.

In an embodiment, B is H.

In a further embodiment, the targeting sequence comprises a sequence selected from the group consisting of:

i)
SEQ ID NO: 5
(CCA GAA GGA AGG BCG AGA AAA GC);
ii)
SEQ ID NO: 6
(CCA GAA GGA AGB GCG AGA AAA GC);
iii)
SEQ ID NO: 7
(CCA GAA GGA ABG GCG AGA AAA GC);
iv)
SEQ ID NO: 8
(CCA GAA GGA AGB BCG AGA AAA GC);
v)
SEQ ID NO: 9
(CCA GAA GGA ABB GCG AGA AAA GC);
vi)
SEQ ID NO: 10
(CCA GAA GGA ABG BCG AGA AAA GC);
vii)
SEQ ID NO: 17
(GCA CTC ACB GGG CTC TCA AAG CAG C);
viii)
SEQ ID NO: 18
(GCA CTC ACG BGG CTC TCA AAG CAG C);
ix)
SEQ ID NO: 19
(GCA CTC ACG GBG CTC TCA AAG CAG C);
x)
SEQ ID NO: 20
(GCA CTC ACG GGB CTC TCA AAG CAG C);
xi)
SEQ ID NO: 21
(GCA CTC ACB BGG CTC TCA AAG CAG C);
xii)
SEQ ID NO: 22
(GCA CTC ACG BBG CTC TCA AAG CAG C);
xiii)
SEQ ID NO: 23
(GCA CTC ACG GBB CTC TCA AAG CAG C);
and
xiv)
SEQ ID NO: 24
(GGC GGC ACT CAC GBB GCT CTC AAA G).

In an embodiment, B is H.

In an embodiment, the conjugate is a pharmaceutically acceptable salt thereof, and at least one pharmaceutically acceptable carrier.

In an embodiment, provided herein is a method of treating a disease in a subject in need thereof, the method comprising administering a therapeutically effective amount of the conjugate or the pharmaceutical composition to the subject.

In an embodiment, the disease is Pompe disease. In an embodiment, the subject is a human. In a further embodiment, the human is a child. In another embodiment, the human is an adult.

### III. Oligomer Chemistry Features

Also provided herein are antisense oligomers, wherein the antisense oligomer is a modified antisense oligomer. Examples of modified antisense oligomers include, without limitation, morpholino oligomers, phosphorothioate modified oligomers, 2′-O-methyl modified oligomers, peptide nucleic acid (PNA), locked nucleic acid (LNA), phosphorothioate oligomers, 2′-O-MOE modified oligomers, 2′-fluoro-modified oligomer, 2′-0,4′-C-ethylene-bridged nucleic acids (ENAs), tricyclo-DNAs, tricyclo-DNA phosphorothioate subunits, 2′-O-[2-(N-methylcarbamoyl)ethyl] modified oligomers, including combinations of any of the foregoing. Phosphorothioate and 2′-O-Me-modified chemistries can be combined to generate a 2′-O-Me-phosphorothioate backbone. See, e.g., PCT Publication Nos. WO/2013/112053 and WO/2009/008725, which are hereby incorporated by reference in their entireties.

In some embodiments, the nucleobases of the modified antisense oligomer are linked to morpholino ring structures, wherein the morpholino ring structures are joined by phosphorous-containing intersubunit linkages joining a morpholino nitrogen of one ring structure to a 5′ exocyclic carbon of an adjacent ring structure.

In some embodiments, the nucleobases of the antisense oligomer are linked to a peptide nucleic acid (PNA), wherein the phosphate-sugar polynucleotide backbone is replaced by a flexible pseudo-peptide polymer to which the nucleobases are linked. In some aspects, at least one of the nucleobases of the antisense oligomer is linked to a locked nucleic acid (LNA), wherein the locked nucleic acid structure is a nucleotide analog that is chemically modified where the ribose moiety has an extra bridge connecting the 2′ oxygen and the 4′ carbon.

In some embodiments, at least one of the nucleobases of the antisense oligomer is linked to a bridged nucleic acid (BNA), wherein the sugar conformation is restricted or locked by the introduction of an additional bridged structure to the furanose skeleton. In some aspects, at least one of the nucleobases of the antisense oligomer is linked to a 2′-O,4′-C-ethylene-bridged nucleic acid (ENA).

In some embodiments, the modified antisense oligomer may contain unlocked nucleic acid (UNA) subunits. UNAs and UNA oligomers are an analogue of RNA in which the C2′-C3′ bond of the subunit has been cleaved.

In some embodiments, the modified antisense oligomer contains one or more phosphorothioates (or S-oligos), in which one of the nonbridging oxygens is replaced by a sulfur. In some aspects, the modified antisense oligomer contains one or more 2′ O-Methyl, 2′ O-MOE, MCE, and 2′-F in which the 2′-OH of the ribose is substituted with a methyl, methoxy ethyl, 2-(N-methylcarbamoyl)ethyl, or fluoro group, respectively.

In some embodiments, the modified antisense oligomer is a tricyclo-DNA (tc-DNA) which is a constrained DNA analog in which each nucleotide is modified by the introduction of a cyclopropane ring to restrict conformational flexibility of the backbone and to optimize the backbone geometry of the torsion angle g.

In some embodiments, at least one of the nucleobases of the antisense oligomer is linked to a bridged nucleic acid (BNA), wherein the sugar conformation is restricted or locked by the introduction of an additional bridged structure to the furanose skeleton. In some aspects, at least one of the nucleobases of the antisense oligomer is linked to a 2′-0,4′-C-ethylene-bridged nucleic acid (ENA). In such aspects, each nucleobase which is linked to a BNA or ENA comprises a 5-methyl group. Exemplary embodiments of oligomer chemistries of the disclosure are further described below.

### 1. Peptide Nucleic Acids (PNAs)

Peptide nucleic acids (PNAs) are analogs of DNA in which the backbone is structurally homomorphous with a deoxyribose backbone, consisting of N-(2-aminoethyl) glycine units to which pyrimidine or purine bases are attached. PNAs containing natural pyrimidine and purine bases hybridize to complementary oligomers obeying Watson-Crick base-pairing rules, and mimic DNA in terms of base-pair recognition. The backbone of PNAs is formed by peptide bonds rather than phosphodiester bonds, making them well-suited for antisense applications (see structure below). The backbone is uncharged, resulting in PNA/DNA or PNA/RNA duplexes that exhibit greater than normal thermal stability. PNAs are not recognized by nucleases or proteases. A non-limiting example of a PNA is depicted below.

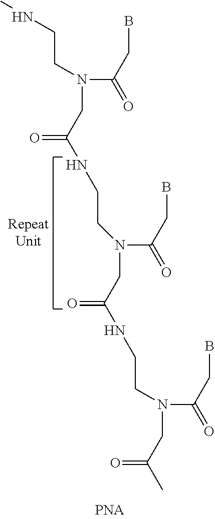

Despite a radical structural change to the natural structure, PNAs are capable of sequence-specific binding in a helix form to DNA or RNA. Characteristics of PNAs include a high binding affinity to complementary DNA or RNA, a destabilizing effect caused by single-base mismatch, resistance to nucleases and proteases, hybridization with DNA or RNA independent of salt concentration, and triplex formation with homopurine DNA. PANAGENE™ has developed its proprietary Bts PNA monomers (Bts; benzothiazole-2-sulfonyl group) and proprietary oligomerization process. The PNA oligomerization using Bts PNA monomers is composed of repetitive cycles of deprotection, coupling, and capping. PNAs can be produced synthetically using any technique known in the art. See, e.g., U.S. Pat. Nos. 6,969,766; 7,211,668; 7,022,851; 7,125,994; 7,145,006; and 7,179,896. See also U.S. Pat. Nos. 5,539,082; 5,714,331; and 5,719,262 for the preparation of PNAs. Further teaching of PNA compounds can be found in Nielsen et al., Science, 254:1497-1500, 1991. Each of the foregoing is incorporated by reference in its entirety.

### 2. Locked Nucleic Acids (LNAs)

Antisense oligomers may also contain “locked nucleic acid” subunits (LNAs). “LNAs” are a member of a class of modifications called bridged nucleic acid (BNA). BNA is characterized by a covalent linkage that locks the conformation of the ribose ring in a C30-endo (northern) sugar pucker. For LNA, the bridge is composed of a methylene between the 2′-0 and the 4′-C positions. LNA enhances backbone preorganization and base stacking to increase hybridization and thermal stability.

The structures of LNAs can be found, for example, in Wengel, et al., Chemical Communications (1998) 455; Koshkin et al., Tetrahedron (1998) 54:3607; Jesper Wengel, Accounts of Chem. Research (1999) 32:301; Obika, et al., Tetrahedron Letters (1997) 38:8735; Obika, et al., Tetrahedron Letters (1998) 39:5401; and Obika, et al., Bioorganic Medicinal Chemistry (2008) 16:9230, which are hereby incorporated by reference in their entirety. A non-limiting example of an LNA is depicted below.

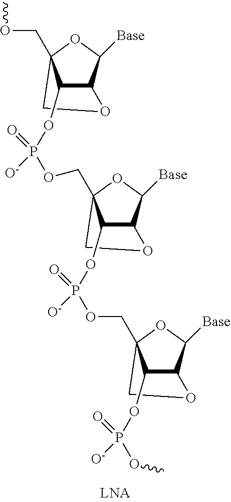

Antisense oligomers of the disclosure may incorporate one or more LNAs; in some cases, the antisense oligomers may be entirely composed of LNAs. Methods for the synthesis of individual LNA nucleoside subunits and their incorporation into oligomers are described, for example, in U.S. Pat. Nos. 7,572,582; 7,569,575; 7,084,125; 7,060,809; 7,053,207; 7,034,133; 6,794,499; and 6,670,461; each of which is incorporated by reference in its entirety. Typical intersubunit linkers include phosphodiester and phosphorothioate moieties; alternatively, non-phosphorous-containing linkers may be employed. Further embodiments include an LNA containing antisense oligomer where each LNA subunit is separated by a DNA subunit. Certain antisense oligomers are composed of alternating LNA and DNA subunits where the intersubunit linker is phosphorothioate.

### 3. Ethylene-Bridged Nucleic Acids (ENAs)

2′O,4′C-ethylene-bridged nucleic acids (ENAs) are another member of the class of BNAs. A non-limiting example is depicted below.

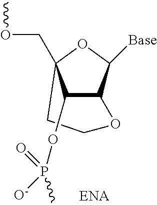

ENA oligomers and their preparation are described in Obika et al., Tetrahedron Lett (1997) 38 (50): 8735, which is hereby incorporated by reference in its entirety. Antisense oligomers of the disclosure may incorporate one or more ENA subunits.

### 4. Unlocked nucleic acid (UNA)

Antisense oligomers may also contain unlocked nucleic acid (UNA) subunits. UNAs and UNA oligomers are an analogue of RNA in which the C2′-C3′ bond of the subunit has been cleaved. Whereas LNA is conformationally restricted (relative to DNA and RNA), UNA is very flexible. UNAs are disclosed, for example, in WO 2016/070166. A non-limiting example of an UNA is depicted below.

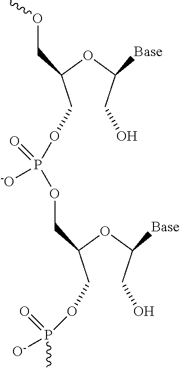

Typical intersubunit linkers include phosphodiester and phosphorothioate moieties, alternatively, non-phosphorous containing linkers may be employed.

### 5. Phosphorothioates

“Phosphorothioates” (or S-oligos) are a variant of normal DNA in which one of the nonbridging oxygens is replaced by a sulfur. A non-limiting example of a phosphorothioate is depicted below.

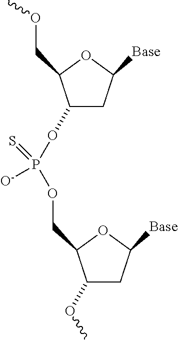

The sulfurization of the internucleotide bond reduces the action of endo- and exonucleases including 5′ to 3′ and 3′ to 5′ DNA POL 1 exonuclease, nucleases S1 and P1, RNases, serum nucleases, and snake venom phosphodiesterase. Phosphorothioates are made by two principal routes: by the action of a solution of elemental sulfur in carbon disulfide on a hydrogen phosphonate, or by the method of sulfurizing phosphite triesters with either tetraethylthiuram disulfide (TETD) or 3H-1, 2-benzodithiol-3-one 1,1-dioxide (BDTD) (see, e.g., lyer et al., J. Org. Chem. 55, 4693-4699, 1990, which is hereby incorporated by reference in its entirety). The latter methods avoid the problem of elemental sulfur's insolubility in most organic solvents and the toxicity of carbon disulfide. The TETD and BDTD methods also yield higher purity phosphorothioates.

### 6. Tricyclo-DNAs and Tricyclo-Phosphorothioate Subunits

Tricyclo-DNAs (tc-DNA) are a class of constrained DNA analogs in which each nucleotide is modified by the introduction of a cyclopropane ring to restrict conformational flexibility of the backbone and to optimize the backbone geometry of the torsion angle γ. Homobasic adenine- and thymine-containing tc-DNAs form extraordinarily stable A-T base pairs with complementary RNAs. Tricyclo-DNAs and their synthesis are described in International Patent Application Publication No. WO 2010/115993, which is hereby incorporated by reference in its entirety. Antisense oligomers of the disclosure may incorporate one or more tricycle-DNA subunits; in some cases, the antisense oligomers may be entirely composed of tricycle-DNA subunits.

Tricyclo-phosphorothioate subunits are tricyclo-DNA subunits with phosphorothioate intersubunit linkages. Tricyclo-phosphorothioate subunits and their synthesis are described in International Patent Application Publication No. WO 2013/053928, which is hereby incorporated by reference in its entirety. Antisense oligomers of the disclosure may incorporate one or more tricycle-DNA subunits; in some cases, the antisense oligomers may be entirely composed of tricycle-DNA subunits. A non-limiting example of a tricycle-DNA/tricycle-phosphorothioate subunit is depicted below.

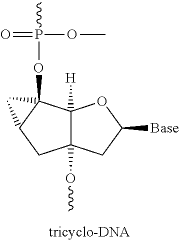

### 7. 2′-O-Methyl, 2′-O-MOE, and 2′-F Oligomers

“2′-O-Me oligomer” molecules carry a methyl group at the 2′-OH residue of the ribose molecule. 2′-O-Me-RNAs show the same (or similar) behavior as DNA but are protected against nuclease degradation. 2′-O-Me-RNAs can also be combined with phosphorothioate oligomers (PTOs) for further stabilization. 2′O-Me oligomers (phosphodiester or phosphorothioate) can be synthesized according to routine techniques in the art (see, e.g., Yoo et al., Nucleic Acids Res. 32:2008-16, 2004, which is hereby incorporated by reference in its entirety). A non-limiting example of a 2′-O-Me oligomer is depicted below.

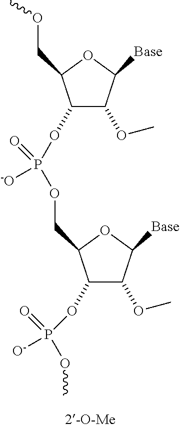

2′-O-Methoxyethyl Oligomers (2′-O-MOE) carry a methoxyethyl group at the 2′-OH residue of the ribose molecule and are discussed in Martin et al., Helv. Chim. Acta, 78, 486-504, 1995, which is hereby incorporated by reference in its entirety. A non-limiting example of a 2′-O-MOE subunit is depicted below.

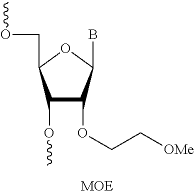

2′-Fluoro (2′-F) oligomers have a fluoro radical in at the 2′ position in place of the 2′-OH. A non-limiting example of a 2′-F oligomer is depicted below.

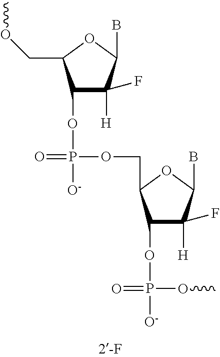

2′-fluoro oligomers are further described in WO 2004/043977, which is hereby incorporated by reference in its entirety.

2′-O-Methyl, 2′-O-MOE, and 2′-F oligomers may also comprise one or more phosphorothioate (PS) linkages as depicted below.

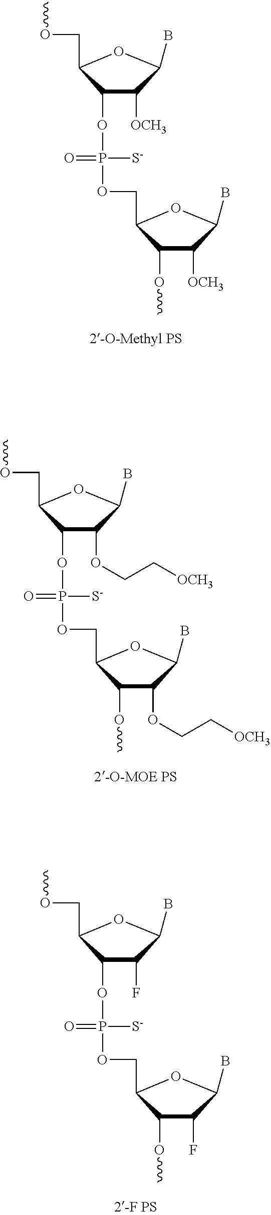

Additionally, 2′-O-Methyl, 2′-O-MOE, and 2′-F oligomers may comprise PS intersubunit linkages throughout the oligomer, for example, as in the 2′-O-methyl PS oligomer drisapersen depicted below.

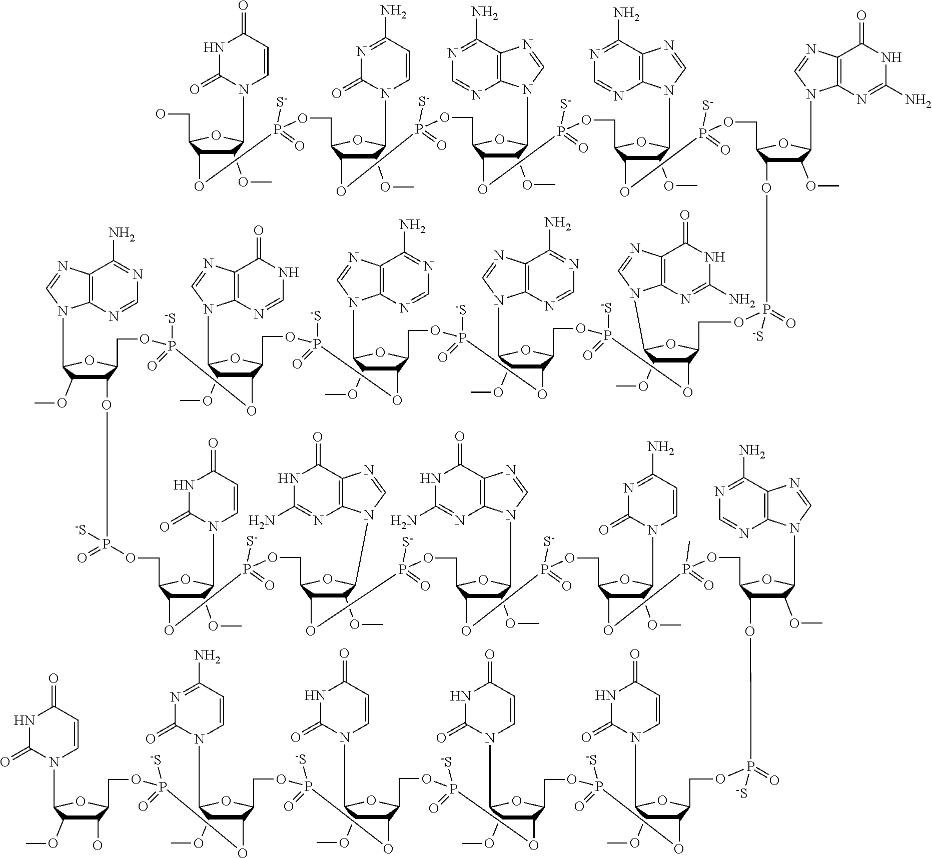

Alternatively, 2′-O-Methyl, 2′-O-MOE, and/or 2′-F oligomers may comprise PS linkages at the ends of the oligomer, as depicted below:

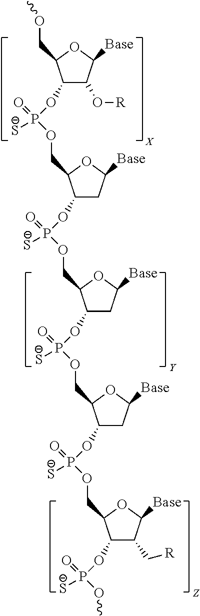

where:
R is CH₂CH₂OCH₃ (methoxyethyl or MOE), and
X, Y, and Z denote the number of nucleotides contained within each of the designated 5′-wing, central gap, and 3′-wing regions, respectively.

Antisense oligomers of the disclosure may incorporate one or more 2′-O-Methyl, 2′-O-MOE, and 2′-F subunits and may utilize any of the intersubunit linkages described here. In some instances, an antisense oligomer of the disclosure may be composed of entirely 2′-O-Methyl, 2′-O-MOE, or 2′-F subunits. One embodiment of the antisense oligomers of the disclosure is composed entirely of 2′-O-methyl subunits.

### 8. 2′-O-[2-(N-methylcarbamoyl)ethyl] Oligomers (MCEs)

MCEs are another example of 2′-0 modified ribonucleosides useful in the antisense oligomers of the disclosure. Here, the 2′-OH is derivatized to a 2-(N-methylcarbamoyl)ethyl moiety to increase nuclease resistance. A non-limiting example of an MCE oligomer is depicted below.

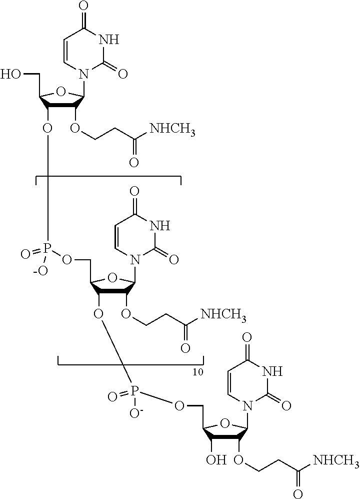

MCEs and their synthesis are described in Yamada et al., J. Org. Chem. (2011) 76(9):3042-53, which is hereby incorporated by reference in its entirety. Antisense oligomers of the disclosure may incorporate one or more MCE subunits.

### 9. Stereo-Specific Oligomers

Stereo-specific oligomers are those in which the stereo chemistry of each phosphorous-containing linkage is fixed by the method of synthesis such that a substantially stereo-pure oligomer is produced. A non-limiting example of a stereo-specific oligomer is depicted below.

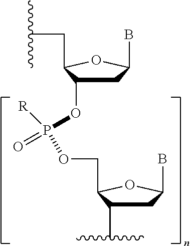

In the above example, each phosphorous of the oligomer has the same stereo configuration. Additional examples include the oligomers described herein. For example, LNAs, ENAs, Tricyclo-DNAs, MCEs, 2′-O-Methyl, 2′-O-MOE, 2′-F, and morpholino-based oligomers can be prepared with stereo-specific phosphorous-containing internucleoside linkages such as, for example, phosphorothioate, phosphodiester, phosphoramidate, phosphorodiamidate, or other phosphorous-containing internucleoside linkages. Stereo specific oligomers, methods of preparation, chiral controlled synthesis, chiral design, and chiral auxiliaries for use in the preparation of such oligomers are detailed, for example, in WO2017192664, WO2017192679, WO2017062862, WO2017015575, WO2017015555, WO2015107425, WO2015108048, WO2015108046, WO2015108047, WO2012039448, WO2010064146, WO2011034072, WO2014010250, WO2014012081, WO20130127858, and WO2011005761, each of which is hereby incorporated by reference in its entirety.

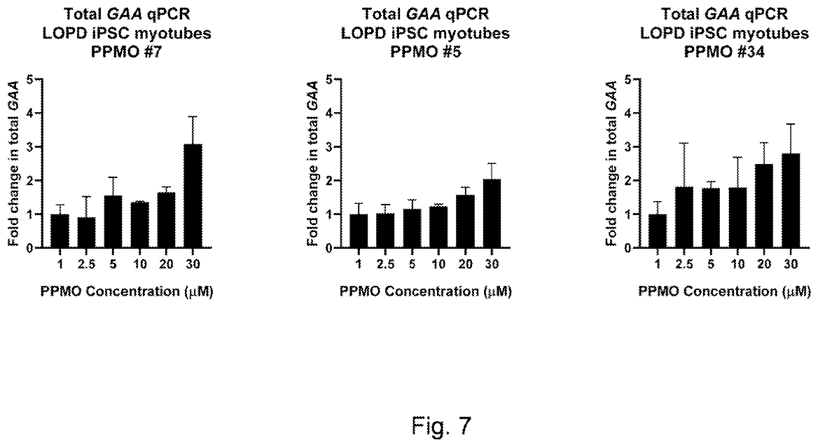

Stereo-specific oligomers can have phosphorous-containing internucleoside linkages in an R_{P} or S_{P} configuration. Chiral phosphorous-containing linkages in which the stereo configuration of the linkages is controlled is referred to as “stereopure,” while chiral phosphorous-containing linkages in which the stereo configuration of the linkages is uncontrolled is referred to as “stereorandom.” In certain embodiments, the oligomers of the disclosure comprise a plurality of stereopure and stereorandom linkages, such that the resulting oligomer has stereopure subunits at pre-specified positions of the oligomer. An example of the location of the stereopure subunits is provided in international patent application publication number WO 2017/062862 A2 in FIGS. 7A and 7B. In an embodiment, all the chiral phosphorous-containing linkages in an oligomer are stereorandom. In an embodiment, all the chiral phosphorous-containing linkages in an oligomer are stereopure.

In an embodiment of an oligomer with n chiral phosphorous-containing linkages (where n is an integer of 1 or greater), all n of the chiral phosphorous-containing linkages in the oligomer are stereorandom. In an embodiment of an oligomer with n chiral phosphorous-containing linkages (where n is an integer of 1 or greater), all n of the chiral phosphorous-containing linkages in the oligomer are stereopure. In an embodiment of an oligomer with n chiral phosphorous-containing linkages (where n is an integer of 1 or greater), at least 10% (to the nearest integer) of the n phosphorous-containing linkages in the oligomer are stereopure. In an embodiment of an oligomer with n chiral phosphorous-containing linkages (where n is an integer of 1 or greater), at least 20% (to the nearest integer) of the n phosphorous-containing linkages in the oligomer are stereopure. In an embodiment of an oligomer with n chiral phosphorous-containing linkages (where n is an integer of 1 or greater), at least 30% (to the nearest integer) of the n phosphorous-containing linkages in the oligomer are stereopure. In an embodiment of an oligomer with n chiral phosphorous-containing linkages (where n is an integer of 1 or greater), at least 40% (to the nearest integer) of the n phosphorous-containing linkages in the oligomer are stereopure. In an embodiment of an oligomer with n chiral phosphorous-containing linkages (where n is an integer of 1 or greater), at least 50% (to the nearest integer) of the n phosphorous-containing linkages in the oligomer are stereopure. In an embodiment of an oligomer with n chiral phosphorous-containing linkages (where n is an integer of 1 or greater), at least 60% (to the nearest integer) of the n phosphorous-containing linkages in the oligomer are stereopure. In an embodiment of an oligomer with n chiral phosphorous-containing linkages (where n is an integer of 1 or greater), at least 70% (to the nearest integer) of the n phosphorous-containing linkages in the oligomer are stereopure. In an embodiment of an oligomer with n chiral phosphorous-containing linkages (where n is an integer of 1 or greater), at least 80% (to the nearest integer) of the n phosphorous-containing linkages in the oligomer are stereopure. In an embodiment of an oligomer with n chiral phosphorous-containing linkages (where n is an integer of 1 or greater), at least 90% (to the nearest integer) of the n phosphorous-containing linkages in the oligomer are stereopure.

In an embodiment of an oligomer with n chiral phosphorous-containing linkages (where n is an integer of 1 or greater), the oligomer contains at least 2 contiguous stereopure phosphorous-containing linkages of the same stereo orientation (i.e. either S_{P} or R_{P}). In an embodiment of an oligomer with n chiral phosphorous-containing linkages (where n is an integer of 1 or greater), the oligomer contains at least 3 contiguous stereopure phosphorous-containing linkages of the same stereo orientation (i.e. either S_{P} or R_{P}). In an embodiment of an oligomer with n chiral phosphorous-containing linkages (where n is an integer of 1 or greater), the oligomer contains at least 4 contiguous stereopure phosphorous-containing linkages of the same stereo orientation (i.e. either S_{P} or R_{P}). In an embodiment of an oligomer with n chiral phosphorous-containing linkages (where n is an integer of 1 or greater), the oligomer contains at least 5 contiguous stereopure phosphorous-containing linkages of the same stereo orientation (i.e. either S_{P} or R_{P}). In an embodiment of an oligomer with n chiral phosphorous-containing linkages (where n is an integer of 1 or greater), the oligomer contains at least 6 contiguous stereopure phosphorous-containing linkages of the same stereo orientation (i.e. either S_{P} or R_{P}). In an embodiment of an oligomer with n chiral phosphorous-containing linkages (where n is an integer of 1 or greater), the oligomer contains at least 7 contiguous stereopure phosphorous-containing linkages of the same stereo orientation (i.e. either S_{P} or R_{P}). In an embodiment of an oligomer with n chiral phosphorous-containing linkages (where n is an integer of 1 or greater), the oligomer contains at least 8 contiguous stereopure phosphorous-containing linkages of the same stereo orientation (i.e. either S_{P} or R_{P}). In an embodiment of an oligomer with n chiral phosphorous-containing linkages (where n is an integer of 1 or greater), the oligomer contains at least 9 contiguous stereopure phosphorous-containing linkages of the same stereo orientation (i.e. either S_{P} or R_{P}). In an embodiment of an oligomer with n chiral phosphorous-containing linkages (where n is an integer of 1 or greater), the oligomer contains at least 10 contiguous stereopure phosphorous-containing linkages of the same stereo orientation (i.e. either S_{P} or R_{P}). In an embodiment of an oligomer with n chiral phosphorous-containing linkages (where n is an integer of 1 or greater), the oligomer contains at least 11 contiguous stereopure phosphorous-containing linkages of the same stereo orientation (i.e. either S_{P} or R_{P}). In an embodiment of an oligomer with n chiral phosphorous-containing linkages (where n is an integer of 1 or greater), the oligomer contains at least 12 contiguous stereopure phosphorous-containing linkages of the same stereo orientation (i.e. either S_{P} or R_{P}). In an embodiment of an oligomer with n chiral phosphorous-containing linkages (where n is an integer of 1 or greater), the oligomer contains at least 13 contiguous stereopure phosphorous-containing linkages of the same stereo orientation (i.e. either S_{P} or R_{P}). In an embodiment of an oligomer with n chiral phosphorous-containing linkages (where n is an integer of 1 or greater), the oligomer contains at least 14 contiguous stereopure phosphorous-containing linkages of the same stereo orientation (i.e. either S_{P} or R_{P}). In an embodiment of an oligomer with n chiral phosphorous-containing linkages (where n is an integer of 1 or greater), the oligomer contains at least 15 contiguous stereopure phosphorous-containing linkages of the same stereo orientation (i.e. either S_{P} or R_{P}). In an embodiment of an oligomer with n chiral phosphorous-containing linkages (where n is an integer of 1 or greater), the oligomer contains at least 16 contiguous stereopure phosphorous-containing linkages of the same stereo orientation (i.e. either S_{P} or R_{P}). In an embodiment of an oligomer with n chiral phosphorous-containing linkages (where n is an integer of 1 or greater), the oligomer contains at least 17 contiguous stereopure phosphorous-containing linkages of the same stereo orientation (i.e. either S_{P} or R_{P}). In an embodiment of an oligomer with n chiral phosphorous-containing linkages (where n is an integer of 1 or greater), the oligomer contains at least 18 contiguous stereopure phosphorous-containing linkages of the same stereo orientation (i.e. either S_{P} or R_{P}). In an embodiment of an oligomer with n chiral phosphorous-containing linkages (where n is an integer of 1 or greater), the oligomer contains at least 19 contiguous stereopure phosphorous-containing linkages of the same stereo orientation (i.e. either S_{P} or R_{P}). In an embodiment of an oligomer with n chiral phosphorous-containing linkages (where n is an integer of 1 or greater), the oligomer contains at least 20 contiguous stereopure phosphorous-containing linkages of the same stereo orientation (i.e. either S_{P} or R_{P}).

In an embodiment of an oligomer with n chiral phosphorous-containing linkages (where n is an integer of 1 or greater), the oligomer contains at least 2 contiguous stereopure phosphorous-containing linkages of the same stereo orientation (i.e. either S_{P} or R_{P}) and at least 2 contiguous stereopure phosphorous-containing linkages of the other stereo orientation. For example, the oligomer can contain at least 2 contiguous stereopure phosphorous-containing linkages of the S_{P} orientation and at least 2 contiguous stereopure phosphorous-containing linkages of the R_{P} orientation.

In an embodiment of an oligomer with n chiral phosphorous-containing linkages (where n is an integer of 1 or greater), the oligomer contains at least 2 contiguous stereopure phosphorous-containing linkages of the same stereo orientation in an alternating pattern. For example, the oligomer can contain the following in order: 2 or more R_{P}, 2 or more S_{P}, and 2 or more R_{P}, etc.

### 10. Morpholino Oligomers

Exemplary embodiments of the disclosure relate to phosphorodiamidate morpholino oligomers of the following general structure:

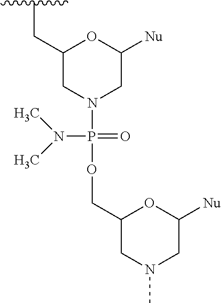

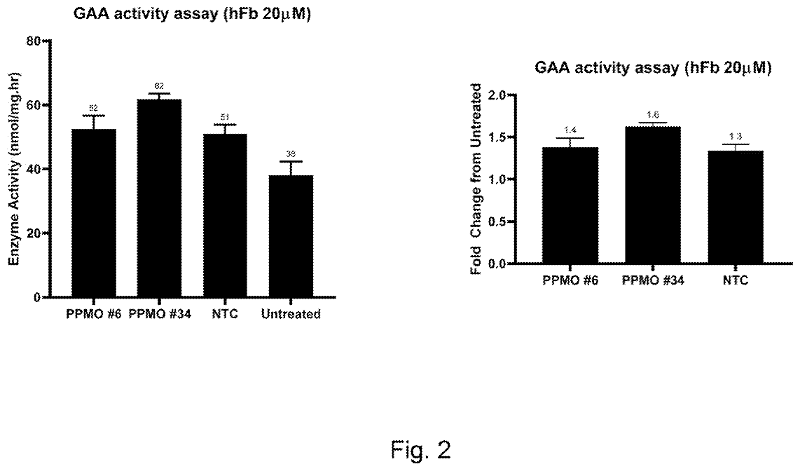

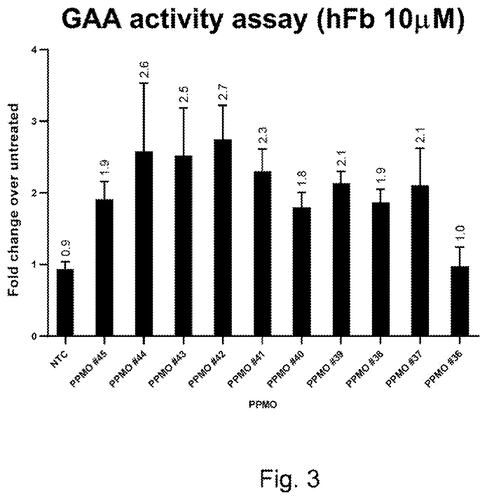

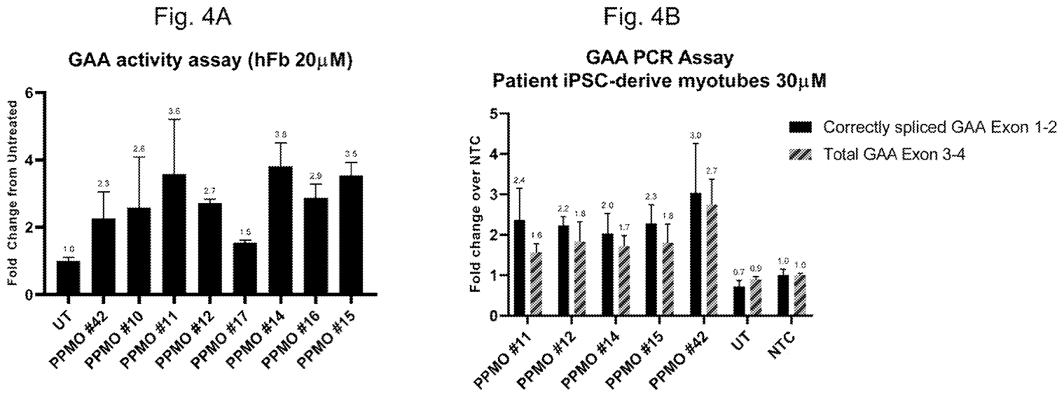

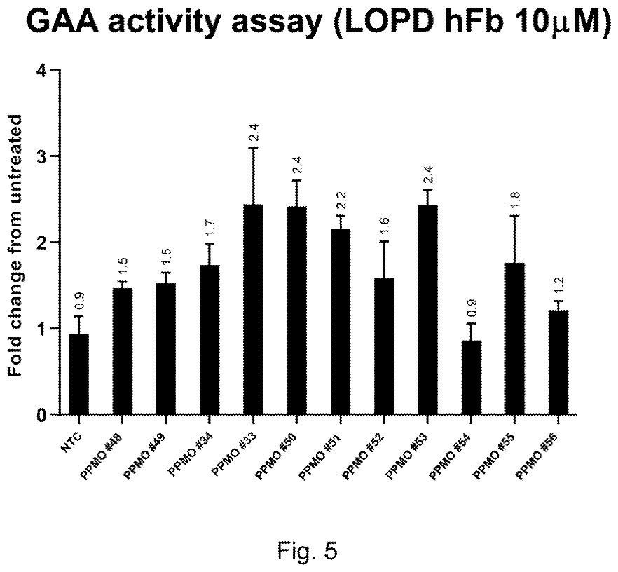

and as described in FIG. 2 of Summerton, J., et al., Antisense & Nucleic Acid Drug Development, 7: 187-195 (1997). Morpholinos as described herein are intended to cover all stereoisomers and tautomers of the foregoing general structure. The synthesis, structures, and binding characteristics of morpholino oligomers are detailed in U.S. Pat. Nos. 5,698,685; 5,217,866; 5,142,047; 5,034,506; 5,166,315; 5,521,063; 5,506,337; 8,076,476; and 8,299,206, all of which are incorporated herein by reference.

In certain embodiments, a morpholino is conjugated at the 5′ or 3′ end of the oligomer with a “tail” moiety to increase its stability and/or solubility. Exemplary tails include:

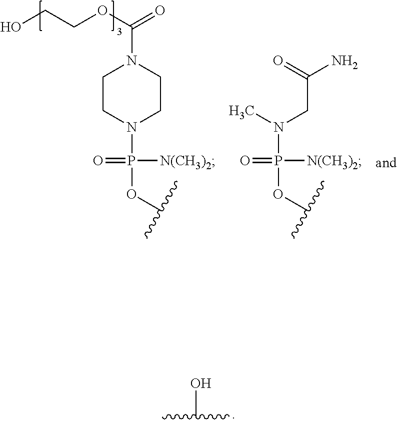

In various aspects, the disclosure provides antisense oligomers according to Formula (IV), or a pharmaceutically acceptable salt thereof.

In an embodiment, the targeting sequence complementary to a target region within intron 1 (SEQ ID NO: 1) of a pre-mRNA of human acid alpha glucosidase (GAA) gene, wherein at least one subunit is an abasic subunit. In another embodiment, the target region comprises a sequence selected from the group consisting of SEQ ID NO: 2 (GAA-IVS1(-189-167)) and SEQ ID NO: 3 (GAA-IVS1(-80-24)). In a further embodiment, the target region comprises the sequence set forth as SEQ ID NO: 2. In another embodiment, the target region comprises the sequence set forth as SEQ ID NO: 3.

In an embodiment, target region is selected from GAA-IVS1(-189-167), GAA-IVS1(-80-56), GAA-IVS1(-76-52), GAA-IVS1(-74-55), GAA-IVS1(-72-48), GAA-IVS1(-71-47), GAA-IVS1(-70-46), GAA-IVS1(-69-45), GAA-IVS1(-66-42), GAA-IVS1(-65-41), and GAA-IVS1(-49-24). In a further embodiment, the target region is GAA-IVS1(-189-167). In another embodiment, the targeting region is GAA-IVS1(-72,-48). In still another embodiment, the targeting region is GAA-IVS1(-71,-47). In yet another embodiment, the targeting region is GAA-IVS1(-70,-46). In an embodiment, the targeting region is GAA-IVS1(-69-45). In another embodiment, the targeting region is GAA-IVS1(-65,-41). In still another embodiment, the targeting region is GAA-IVS1(-66,-42).

In an embodiment, the targeting sequence comprises the sequence CCA GAA GGA AXX XCG AGA AAA GC (SEQ ID NO: 4), wherein each X is independently selected from guanine (G) or is abasic (B), wherein at least one X is B. In another embodiment, the targeting sequence comprises a sequence selected from the group consisting of:

i)
SEQ ID NO: 5
(CCA GAA GGA AGG BCG AGA AAA GC);
ii)
SEQ ID NO: 6
(CCA GAA GGA AGB GCG AGA AAA GC);
iii)
SEQ ID NO: 7
(CCA GAA GGA ABG GCG AGA AAA GC);
iv)
SEQ ID NO: 8
(CCA GAA GGA AGB BCG AGA AAA GC);
v)
SEQ ID NO: 9
(CCA GAA GGA ABB GCG AGA AAA GC);
and
vi)
SEQ ID NO: 10
(CCA GAA GGA ABG BCG AGA AAA GC).

In an embodiment, B is H.

In an embodiment, the targeting sequence comprises SEQ ID NO: 5 (CCA GAA GGA AGG BCG AGA AAA GC). In another embodiment, the targeting sequence comprises SEQ ID NO: 6 (CCA GAA GGA AGB GCG AGA AAA GC). In still another embodiment, the targeting sequence comprises SEQ ID NO: 7 (CCA GAA GGA ABG GCG AGA AAA GC). In yet another embodiment, the targeting sequence comprises SEQ ID NO: 8 (CCA GAA GGA AGB BCG AGA AAA GC). In an embodiment, the targeting sequence comprises SEQ ID NO: 9 (CCA GAA GGA ABB GCG AGA AAA GC). In another embodiment, the targeting sequence comprises SEQ ID NO: 10 (CCA GAA GGA ABG BCG AGA AAA GC).

In an embodiment, the targeting sequence consists of the sequence CCA GAA GGA AXX XCG AGA AAA GC (SEQ ID NO: 4). In another embodiment, the targeting sequence consists of SEQ ID NO: 5 (CCA GAA GGA AGG BCG AGA AAA GC). In still another embodiment, the targeting sequence consists of SEQ ID NO: 6 (CCA GAA GGA AGB GCG AGA AAA GC). In yet another embodiment, the targeting sequence consists of SEQ ID NO: 7 (CCA GAA GGA ABG GCG AGA AAA GC). In an embodiment, the targeting sequence consists of SEQ ID NO: 8 (CCA GAA GGA AGB BCG AGA AAA GC). In another embodiment, the targeting sequence consists of SEQ ID NO: 9 (CCA GAA GGA ABB GCG AGA AAA GC). In still another embodiment, the targeting sequence consists of SEQ ID NO: 10 (CCA GAA GGA ABG BCG AGA AAA GC).

In an embodiment, the target region is selected from the group consisting of GAA-IVS1(-80-56), GAA-IVS1(-76-52), GAA-IVS1(-74-55), GAA-IVS1(-72-48), GAA-IVS1(-71-47), GAA-IVS1(-70-46), GAA-IVS1(-69-45), GAA-IVS1(-66-42), GAA-IVS1(-65-41), and GAA-IVS1(-49-24). In another embodiment, the target region is selected from the group consisting of GAA-IVS1(-72-48), GAA-IVS1(-71-47), GAA-IVS1(-70-46), GAA-IVS1(-69-45), GAA-IVS1(-66-42), and GAA-IVS1(-65-41).

In an embodiment, the targeting sequence comprises a sequence selected from the group consisting of:

i)
SEQ ID NO: 11
(CTC ACX XXX CTC TCA AAG CAG CTC T);
ii)
SEQ ID NO: 12
(ACT CAC XXX XCT CTC AAA GCA GCT C);
iii)
SEQ ID NO: 13
(CAC TCA CXX XXC TCT CAA AGC AGC T);
iv)
SEQ ID NO: 14
(GCA CTC ACX XXX CTC TCA AAG CAG C);
v)
SEQ ID NO: 15
(GCG GCA CTC ACX XXX CTC TCA AAG C);
vi)
SEQ ID NO: 16
(GGC GGC ACT CAC XXX XCT CTC AAA G);

wherein each X is independently selected from guanine (G) or is abasic (B), wherein at least one X is B. In an embodiment, the targeting sequence is selected from the group consisting of:

i)
SEQ ID NO: 17
(GCA CTC ACB GGG CTC TCA AAG CAG C);
ii)
SEQ ID NO: 18
(GCA CTC ACG BGG CTC TCA AAG CAG C);
iii)
SEQ ID NO: 19
(GCA CTC ACG GBG CTC TCA AAG CAG C);
iv)
SEQ ID NO: 20
(GCA CTC ACG GGB CTC TCA AAG CAG C);
v)
SEQ ID NO: 21
(GCA CTC ACB BGG CTC TCA AAG CAG C);
vi)
SEQ ID NO: 22
(GCA CTC ACG BBG CTC TCA AAG CAG C);
vii)
SEQ ID NO: 23
(GCA CTC ACG GBB CTC TCA AAG CAG C);
and
viii)
SEQ ID NO: 24
(GGC GGC ACT CAC GBB GCT CTC AAA G).

In an embodiment, B is H.

In an embodiment, the targeting sequence comprises SEQ ID NO: 11 (CTC ACX XXX CTC TCA AAG CAG CTC T). In another embodiment, the targeting sequence comprises SEQ ID NO: 12 (ACT CAC XXX XCT CTC AAA GCA GCT C). In still another embodiment, the targeting sequence comprises SEQ ID NO: 13 (CAC TCA CXX XXC TCT CAA AGC AGC T). In yet another embodiment, the targeting sequence comprises SEQ ID NO: 14 (GCA CTC ACX XXX CTC TCA AAG CAG C). In an embodiment, the targeting sequence comprises SEQ ID NO: 15 (GCG GCA CTC ACX XXX CTC TCA AAG C). In another embodiment, the targeting sequence comprises SEQ ID NO: 16 (GGC GGC ACT CAC XXX XCT CTC AAA G). In still another embodiment, the targeting sequence comprises SEQ ID NO: 17 (GCA CTC ACB GGG CTC TCA AAG CAG C). In yet another embodiment, the targeting sequence comprises SEQ ID NO: 18 (GCA CTC ACG BGG CTC TCA AAG CAG C). In an embodiment, the targeting sequence comprises SEQ ID NO: 19 (GCA CTC ACG GBG CTC TCA AAG CAG C). In another embodiment, the targeting sequence comprises SEQ ID NO: 20 (GCA CTC ACG GGB CTC TCA AAG CAG C). In still another embodiment, the targeting sequence comprises SEQ ID NO: 21 (GCA CTC ACB BGG CTC TCA AAG CAG C). In yet another embodiment, the targeting sequence comprises SEQ ID NO: 22 (GCA CTC ACG BBG CTC TCA AAG CAG C). In an embodiment, the targeting sequence comprises SEQ ID NO: 23 (GCA CTC ACG GBB CTC TCA AAG CAG C). In another embodiment, the targeting sequence comprises SEQ ID NO: 24 (GGC GGC ACT CAC GBB GCT CTC AAA G).

In an embodiment, the targeting sequence consists of SEQ ID NO: 11 (CTC ACX XXX CTC TCA AAG CAG CTC T). In another embodiment, the targeting sequence consists of SEQ ID NO: 12 (ACT CAC XXX XCT CTC AAA GCA GCT C). In still another embodiment, the targeting sequence consists of SEQ ID NO: 13 (CAC TCA CXX XXC TCT CAA AGC AGC T). In yet another embodiment, the targeting sequence consists of SEQ ID NO: 14 (GCA CTC ACX XXX CTC TCA AAG CAG C). In an embodiment, the targeting sequence consists of SEQ ID NO: 15 (GCG GCA CTC ACX XXX CTC TCA AAG C). In another embodiment, the targeting sequence consists of SEQ ID NO: 16 (GGC GGC ACT CAC XXX XCT CTC AAA G). In still another embodiment, the targeting sequence consists of SEQ ID NO: 17 (GCA CTC ACB GGG CTC TCA AAG CAG C). In yet another embodiment, the targeting sequence consists of SEQ ID NO: 18 (GCA CTC ACG BGG CTC TCA AAG CAG C). In an embodiment, the targeting sequence consists of SEQ ID NO: 19 (GCA CTC ACG GBG CTC TCA AAG CAG C). In another embodiment, the targeting sequence consists of SEQ ID NO: 20 (GCA CTC ACG GGB CTC TCA AAG CAG C). In still another embodiment, the targeting sequence consists of SEQ ID NO: 21 (GCA CTC ACB BGG CTC TCA AAG CAG C). In yet another embodiment, the targeting sequence consists of SEQ ID NO: 22 (GCA CTC ACG BBG CTC TCA AAG CAG C). In an embodiment, the targeting sequence consists of SEQ ID NO: 23 (GCA CTC ACG GBB CTC TCA AAG CAG C). In another embodiment, the targeting sequence consists of SEQ ID NO: 24 (GGC GGC ACT CAC GBB GCT CTC AAA G).

In some embodiments, an antisense oligomer of the disclosure is according to Formula II:

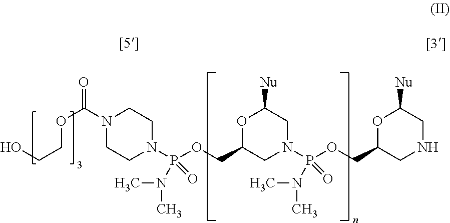

or a pharmaceutically acceptable salt thereof, where each Nu from 1 to n and 5′ to 3′ corresponds to the nucleobases in one of the following:

| | | SEQ |
|---|---|---|
| | | ID |
| Name | Targeting Sequence [5′ to 3′] | NO: |
| GAA-IVS1 | CCA GAA GGA AXX XCG AGA AAA GC | 4 |
| (−189, 167) | | |
| GAA-IVS1 | CTC ACX XXX CTC TCA AAG CAG CTC T | 11 |
| (−72, −48) | | |
| GAA-IVS1 | ACT CAC XXX XCT CTC AAA GCA GCT C | 12 |
| (−71, −47) | | |
| GAA-IVS1 | CAC TCA CXX XXC TCT CAA AGC AGC T | 13 |
| (−70, −46) | | |
| GAA-IVS1 | GCA CTC ACX XXX CTC TCA AAG CAG C | 14 |
| (−69-45) | | |
| GAA-IVS1 | GCG GCA CTC ACX XXX CTC TCA AAG C | 15 |
| (−65, −41) | | |
| GAA-IVS1 | GGC GGC ACT CAC XXX XCT CTC AAA G | 16 |
| (−66, −42) | | |

wherein each X is independently selected from guanine (G) or is abasic (B), wherein at least one X is B. In instances where X is abasic (B), hydrogen is present in place of nucleobases A, C, T, or G.

In an embodiment, B is H.

In an embodiment, the targeting sequence comprises or consists of any one of the sequences:

| | | SEQ |
|---|---|---|
| | | ID |
| Name | Targeting Sequence [5′ to 3′] | NO: |
| GAA-IVS1 | CCA GAA GGA AGG BCG AGA AAA GC | 5 |
| (−189, 167) | | |
| (−179 Abasic) | | |
| GAA-IVS1 | CCA GAA GGA AGB GCG AGA AAA GC | 6 |
| (−189, 167) | | |
| (−178 Abasic) | | |
| GAA-IVS1 | CCA GAA GGA ABG GCG AGA AAA GC | 7 |
| (−189, 167) | | |
| (−177 Abasic) | | |
| GAA-IVS1 | CCA GAA GGA AGB BCG AGA AAA GC | 8 |
| (−189, 167) | | |
| (−178, −179 | | |
| Abasic) | | |
| GAA-IVS1 | CCA GAA GGA ABB GCG AGA AAA GC | 9 |
| (−189, 167) | | |
| (−177, −178 | | |
| Abasic) | | |
| GAA-IVS1 | CCA GAA GGA ABG BCG AGA AAA GC | 10 |
| (−189, 167) | | |
| (−177, −179 | | |
| Abasic) | | |
| GAA-IVS1 | GCA CTC ACB GGG CTC TCA AAG | 17 |
| (-69-45) | CAG C | |
| (G53B) | | |
| GAA-IVS1 | GCA CTC ACG BGG CTC TCA AAG | 18 |
| (−69-45) | CAG C | |
| (G54B) | | |
| GAA-IVS1 | GCA CTC ACG GBG CTC TCA AAG | 19 |
| (−69-45) | CAG C | |
| (G55B) | | |
| GAA-IVS1 | GCA CTC ACG GGB CTC TCA AAG | 20 |
| (−69-45) | CAG C | |
| (G56B) | | |
| GAA-IVS1 | GCA CTC ACB BGG CTC TCA AAG | 21 |
| (−69-45) | CAG C | |
| (G53B G54B) | | |
| GAA-IVS1 | GCA CTC ACG BBG CTC TCA AAG | 22 |
| (−69-45) | CAG C | |
| (G54B G55B) | | |
| GAA-IVS1 | GCA CTC ACG GBB CTC TCA AAG | 23 |
| (−69-45) | CAG C | |
| (G55B G56B) | | |
| GAA-IVS1 | GGC GGC ACT CAC GBB GCT CTC | 24. |
| (−65-41) | AAA G | |
| (G54B G55B) | | |

In an embodiment, B is H.

In some embodiments, an antisense oligomer of Formula (II) is in free base form. In some embodiments, an antisense oligomer of Formula (II) is a pharmaceutically acceptable salt form thereof. In some embodiments, an antisense oligomer of Formula (II) is an HCl (hydrochloric acid) salt thereof. In certain embodiments, the HCl salt is a 1 HCl, 2 HCl, 3 HCl, 4 HCl, 5 HCl, or 6 HCl salt. In certain embodiments, the HCl salt is a 6 HCl salt.

In some embodiments, an antisense oligomer of the disclosure is according to Formula (IIIa):

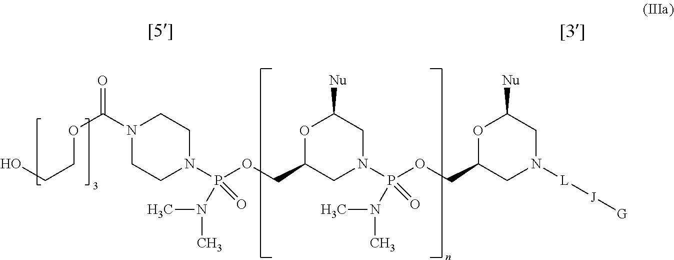

or a pharmaceutically acceptable salt thereof, where each Nu from 1 to n and 5′ to 3′ corresponds to the nucleobases in one of the following:

| | | SEQ |
|---|---|---|
| | | ID |
| Name | Targeting Sequence [5′ to 3′] | NO: |
| GAA-IVS1 | CCA GAA GGA AXX XCG AGA AAA GC | 4 |
| (−189, 167) | | |
| GAA-IVS1 | CTC ACX XXX CTC TCA AAG CAG CTC T | 11 |
| (−72, −48) | | |
| GAA-IVS1 | ACT CAC XXX XCT CTC AAA GCA GCT C | 12 |
| (−71, −47) | | |
| GAA-IVS1 | CAC TCA CXX XXC TCT CAA AGC AGC T | 13 |
| (−70, −46) | | |
| GAA-IVS1 | GCA CTC ACX XXX CTC TCA AAG CAG C | 14 |
| (−69-45) | | |
| GAA-IVS1 | GCG GCA CTC ACX XXX CTC TCA AAG C | 15 |
| (−65, −41) | | |
| GAA-IVS1 | GGC GGC ACT CAC XXX XCT CTC AAA G | 16 |
| (−66, −42) | | |

wherein each X is independently selected from guanine (G) or is abasic (B), wherein at least one X is B. In instances where X is abasic (B), hydrogen is present in place of nucleobases A, C, T, or G.

In an embodiment, B is H.

In an embodiment, the targeting sequence comprises or consists of any one of the sequences:

| | | SEQ |
|---|---|---|
| | | ID |
| Name | Targeting Sequence [5′ to 3′] | NO: |
| GAA-IVS1 | CCA GAA GGA AGG BCG AGA AAA GC | 5 |
| (−189, 167) | | |
| (−179 Abasic) | | |
| GAA-IVS1 | CCA GAA GGA AGB GCG AGA AAA GC | 6 |
| (−189, 167) | | |
| (-178 Abasic) | | |
| GAA-IVS1 | CCA GAA GGA ABG GCG AGA AAA GC | 7 |
| (−189, 167) | | |
| (−177 Abasic) | | |
| GAA-IVS1 | CCA GAA GGA AGB BCG AGA AAA GC | 8 |
| (−189, 167) | | |
| (−178, −179 | | |
| Abasic) | | |
| GAA-IVS1 | CCA GAA GGA ABB GCG AGA AAA GC | 9 |
| (-189, 167) | | |
| (−177, −178 | | |
| Abasic) | | |
| GAA-IVS1 | CCA GAA GGA ABG BCG AGA AAA GC | 10 |
| (−189, 167) | | |
| (−177, −179 | | |
| Abasic) | | |
| GAA-IVS1 | GCA CTC ACB GGG CTC TCA AAG CAG C | 17 |
| (−69-45) | | |
| (G53B) | | |
| GAA-IVS1 | GCA CTC ACG BGG CTC TCA AAG CAG C | 18 |
| (−69-45) | | |
| (G54B) | | |
| GAA-IVS1 | GCA CTC ACG GBG CTC TCA AAG CAG C | 19 |
| (−69-45) | | |
| (G55B) | | |
| GAA-IVS1 | GCA CTC ACG GGB CTC TCA AAG CAG C | 20 |
| (−69-45) | | |
| (G56B) | | |
| GAA-IVS1 | GCA CTC ACB BGG CTC TCA AAG CAG C | 21 |
| (−69-45) | | |
| (G53B G54B) | | |
| GAA-IVS1 | GCA CTC ACG BBG CTC TCA AAG CAG C | 22 |
| (−69-45) | | |
| (G54B G55B) | | |
| GAA-IVS1 | GCA CTC ACG GBB CTC TCA AAG CAG C | 23 |
| (−69-45) | | |
| (G55B G56B) | | |
| GAA-IVS1 | GGC GGC ACT CAC GBB GCT CTC AAA G | 24. |
| (−65-41) | | |
| (G54B G55B) | | |

In an embodiment, B is H.

In some embodiments, an antisense oligomer of Formula (IIIa) is in free base form. In some embodiments, an antisense oligomer of Formula (IIIa) is a pharmaceutically acceptable salt thereof. In some embodiments, an antisense oligomer of Formula (IIIa) is an HCl (hydrochloric acid) salt thereof. In certain embodiments, the HCl salt is a 5 HCl salt. In certain embodiments, the HCl salt is a 6 HCl salt.

In some embodiments, an antisense oligomer of the disclosure is according to Formula (III):

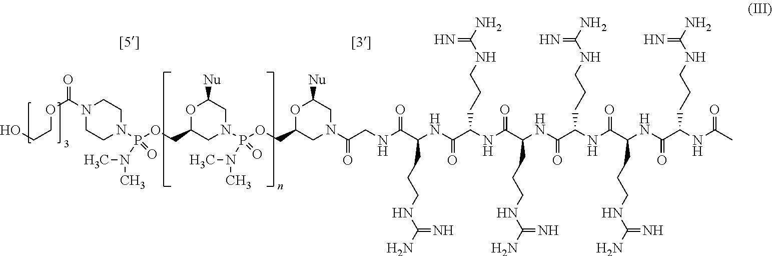

or a pharmaceutically acceptable salt thereof, where each Nu from 1 to n and 5′ to 3′ corresponds to the nucleobases in one of the following:

| | | SEQ |
|---|---|---|
| | | ID |
| Name | Targeting Sequence [5′ to 3′] | NO: |
| GAA-IVS1 | CCA GAA GGA AXX XCG AGA AAA GC | 4 |
| (−189, 167) | | |
| GAA-IVS1 | CTC ACX XXX CTC TCA AAG CAG CTC T | 11 |
| (−72, −48) | | |
| GAA-IVS1 | ACT CAC XXX XCT CTC AAA GCA GCT C | 12 |
| (−71, −47) | | |
| GAA-IVS1 | CAC TCA CXX XXC TCT CAA AGC AGC T | 13 |
| (−70, −46) | | |
| GAA-IVS1 | GCA CTC ACX XXX CTC TCA AAG CAG C | 14 |
| (−69-45) | | |
| GAA-IVS1 | GCG GCA CTC ACX XXX CTC TCA AAG C | 15 |
| (−65, −41) | | |
| GAA-IVS1 | GGC GGC ACT CAC XXX XCT CTC AAA G | 16 |
| (−66, −42) | | |

wherein each X is independently selected from guanine (G) or is abasic (B), wherein at least one X is B. In instances where X is abasic (B), hydrogen is present in place of nucleobases A, C, T, or G.

In an embodiment, B is H.

In an embodiment, the targeting sequence comprises or consists of any one of the sequences:

| | | SEQ |
|---|---|---|
| | | ID |
| Name | Targeting Sequence [5′ to 3′] | NO: |
| GAA-IVS1 | CCA GAA GGA AGG BCG AGA AAA GC | 5 |
| (-189, 167) | | |
| (-179 Abasic) | | |
| GAA-IVS1 | CCA GAA GGA AGB GCG AGA AAA GC | 6 |
| (-189, 167) | | |
| (-178 Abasic) | | |
| GAA-IVS1 | CCA GAA GGA ABG GCG AGA AAA GC | 7 |
| (-189, 167) | | |
| (-177 Abasic) | | |
| GAA-IVS1 | CCA GAA GGA AGB BCG AGA AAA GC | 8 |
| (-189, 167) | | |
| (-178,-179 | | |
| Abasic) | | |
| GAA-IVS1 | CCA GAA GGA ABB GCG AGA AAA GC | 9 |
| (-189, 167) | | |
| (-177,-178 | | |
| Abasic) | | |
| GAA-IVS1 | CCA GAA GGA ABG BCG AGA AAA GC | 10 |
| (-189, 167) | | |
| (-177,-179 | | |
| Abasic) | | |
| GAA-IVS1 | GCA CTC ACB GGG CTC TCA AAG | 17 |
| (-69-45) | CAG C | |
| (G53B) | | |
| GAA-IVS1 | GCA CTC ACG BGG CTC TCA AAG | 18 |
| (-69-45) | CAG C | |
| (G54B) | | |
| GAA-IVS1 | GCA CTC ACG GBG CTC TCA AAG | 19 |
| (-69-45) | CAG C | |
| (G55B) | | |
| GAA-IVS1 | GCA CTC ACG GGB CTC TCA AAG | 20 |
| (-69-45) | CAG C | |
| (G56B) | | |
| GAA-IVS1 | GCA CTC ACB BGG CTC TCA AAG | 21 |
| (-69-45) | CAG C | |
| (G53B G54B) | | |
| GAA-IVS1 | GCA CTC ACG BBG CTC TCA AAG | 22 |
| (-69-45) | CAG C | |
| (G54B G55B) | | |
| GAA-IVS1 | GCA CTC ACG GBB CTC TCA AAG | 23 |
| (-69-45) | CAG C | |
| (G55B G56B) | | |
| GAA-IVS1 | GGC GGC ACT CAC GBB GCT CTC | 24. |
| (-65-41) | AAA G | |
| (G54B G55B) | | |

In an embodiment, B is H.

In some embodiments, an antisense oligomer of Formula (III) is in free base form. In some embodiments, an antisense oligomer of Formula (III) is a pharmaceutically acceptable salt thereof. In some embodiments, an antisense oligomer of Formula (III) is an HCl (hydrochloric acid) salt thereof. In certain embodiments, the HCl salt is a 5 HCl salt. In certain embodiments, the HCl salt is a 6 HCl salt.

In some embodiments, an antisense oligomer of the disclosure is according to Formula (V):

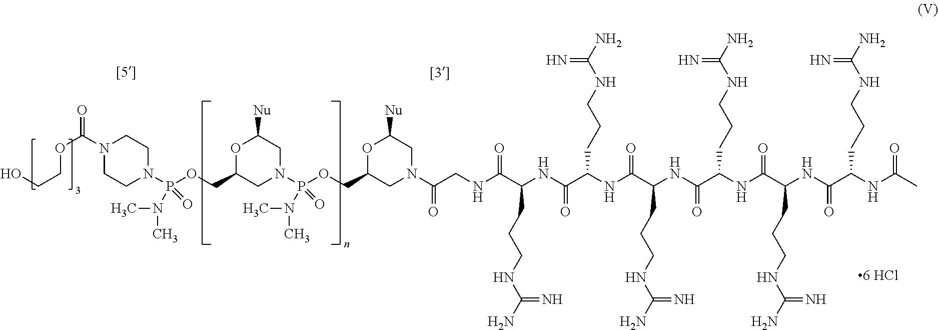

where each Nu from 1 to n and 5′ to 3′ corresponds to the nucleobases in one of the following:

| | | SEQ |
|---|---|---|
| | | ID |
| Name | Targeting Sequence [5′ to 3′] | NO: |
| GAA-IVS1 | CCA GAA GGA AXX XCG AGA AAA GC | 4 |
| (-189, 167) | | |
| GAA-IVS1 | CTC ACX XXX CTC TCA AAG CAG CTC T | 11 |
| (-72, -48) | | |
| GAA-IVS1 | ACT CAC XXX XCT CTC AAA GCA GCT C | 12 |
| (-71, -47) | | |
| GAA-IVS1 | CAC TCA CXX XXC TCT CAA AGC AGC T | 13 |
| (-70, -46) | | |
| GAA-IVS1 | GCA CTC ACX XXX CTC TCA AAG CAG C | 14 |
| (-69-45) | | |
| GAA-IVS1 | GCG GCA CTC ACX XXX CTC TCA AAG C | 15 |
| (-65, -41) | | |
| GAA-IVS1 | GGC GGC ACT CAC XXX XCT CTC AAA G | 16 |
| (-66, -42) | | |

wherein each X is independently selected from guanine (G) or is abasic (B), wherein at least one X is B. In instances where X is abasic (B), hydrogen is present in place of nucleobases A, C, T, or G.

In an embodiment, B is H.

In some aspects of Formula (V), one instance of X is abasic and the other instances of X are each G. In certain aspects, two instances of X are abasic and one instance is G. In some aspects of Formula (V), the first instance of X from 5′ to 3′ is abasic and the other two instances of X are G. In some aspects of Formula (V), the second instance of X from 5′ to 3′ is abasic and the first and third instance of X are G. In certain aspects of Formula of (V), the third instance of X from 5 to 3 is abasic and the first and second instance of X are G.

In some aspects of Formula (V), two instances of X are abasic and the other instance of X is G. In some aspects of Formula (V), the first and second instances of X from 5′ to 3′ are abasic and the third instances of X is G. In some aspects of Formula of (V), the first and third instances of X from 5 to 3 are abasic and the second instances of X is G. In some aspects of Formula of (V), the second and third instances of X from 5′ to 3′ are abasic and the first instances of X is G.

In an embodiment, the targeting sequence comprises or consists of any one of the sequences:

| | | SEQ |
|---|---|---|
| | | ID |
| Name | Targeting Sequence [5′ to 3′] | NO: |
| GAA-IVS1 | CCA GAA GGA AGG BCG AGA AAA GC | 5 |
| (-189, 167) | | |
| (-179 Abasic) | | |
| GAA-IVS1 | CCA GAA GGA AGB GCG AGA AAA GC | 6 |
| (-189, 167) | | |
| (-178 Abasic) | | |
| GAA-IVS1 | CCA GAA GGA ABG GCG AGA AAA GC | 7 |
| (-189, 167) | | |
| (-177 Abasic) | | |
| GAA-IVS1 | CCA GAA GGA AGB BCG AGA AAA GC | 8 |
| (-189, 167) | | |
| (-178, -179 | | |
| Abasic) | | |
| GAA-IVS1 | CCA GAA GGA ABB GCG AGA AAA GC | 9 |
| (-189, 167) | | |
| (-177, -178 | | |
| Abasic) | | |
| GAA-IVS1 | CCA GAA GGA ABG BCG AGA AAA GC | 10 |
| (-189, 167) | | |
| (-177, -179 | | |
| Abasic) | | |
| GAA-IVS1 | GCA CTC ACB GGG CTC TCA AAG | 17 |
| (-69-45) | CAG C | |
| (G53B) | | |
| GAA-IVS1 | GCA CTC ACG BGG CTC TCA AAG | 18 |
| (-69-45) | CAG C | |
| (G54B) | | |
| GAA-IVS1 | GCA CTC ACG GBG CTC TCA AAG | 19 |
| (-69-45) | CAG C | |
| (G55B) | | |
| GAA-IVS1 | GCA CTC ACG GGB CTC TCA AAG | 20 |
| (-69-45) | CAG C | |
| (G56B) | | |
| GAA-IVS1 | GCA CTC ACB BGG CTC TCA AAG | 21 |
| (-69-45) | CAG C | |
| (G53B G54B) | | |
| GAA-IVS1 | GCA CTC ACG BBG CTC TCA AAG | 22 |
| (-69-45) | CAG C | |
| (G54B G55B) | | |
| GAA-IVS1 | GCA CTC ACG GBB CTC TCA AAG | 23 |
| (-69-45) | CAG C | |
| (G55B G56B) | | |
| GAA-IVS1 | GGC GGC ACT CAC GBB GCT CTC | 24. |
| (-65-41) | AAA G | |
| (G54B G55B) | | |

In an embodiment, B is H.

In some embodiments, including, for example, some embodiments of Formula (V), the antisense oligomer is according to Formula (Va):

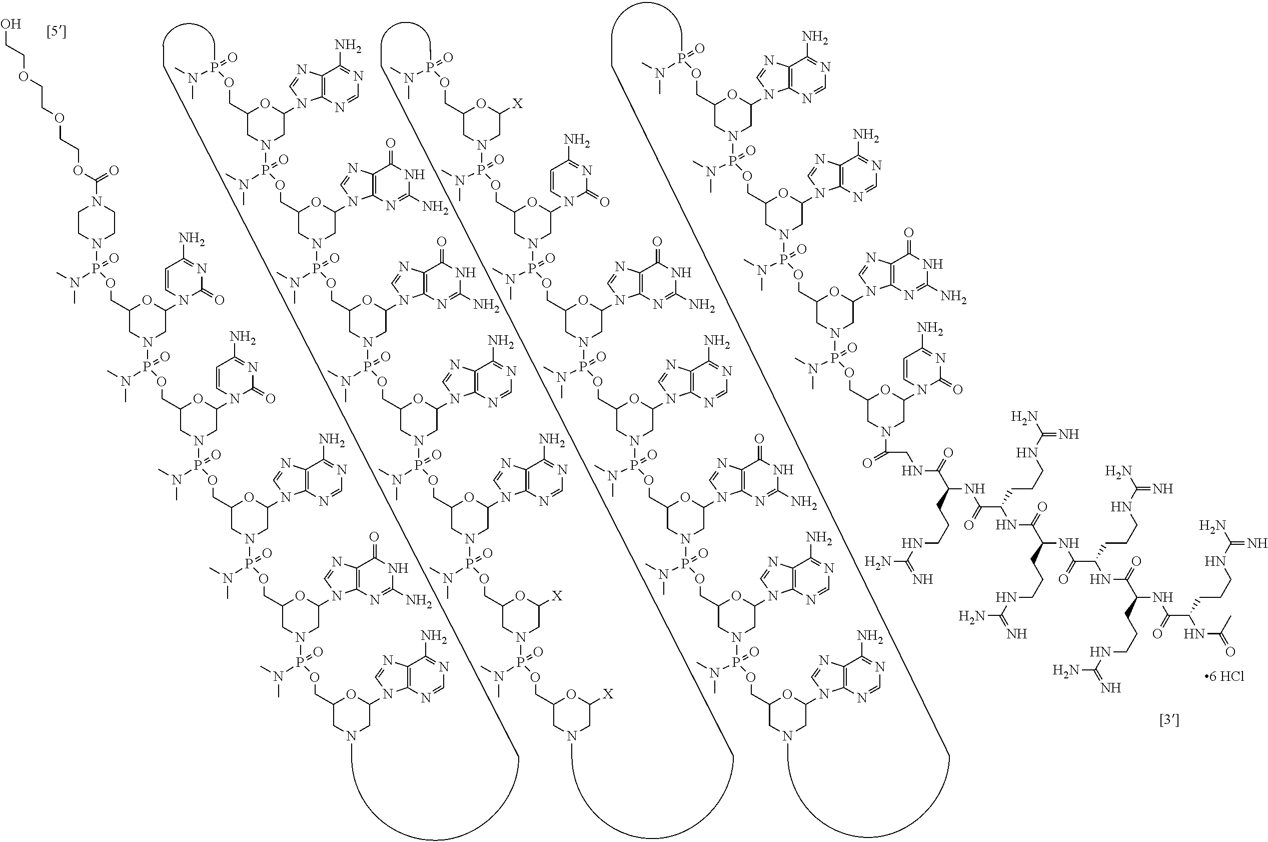

wherein each X is independently selected from guanine (G) or is abasic (B), wherein at least one X is B.

In an embodiment, B is H.

In some aspects of Formula (Va), one instance of X is abasic and the other instances of X are each G. In certain aspects of Formula (Va), two instances of X are abasic and one instance is G. In some aspects of Formula (Va), the first instance of X from 5′ to 3′ is abasic and the other two instances of X are G. In some aspects of Formula (Va), the second instance of X from 5′ to 3′ is abasic and the first and third instance of X are G. In certain aspects of Formula (Va), the third instance of X from 5′ to 3′ is abasic and the first and second instance of X are G.

In some aspects of Formula (Va), two instances of X are abasic and the other instance of X is G. In some aspects of Formula (Va), the first and second instances of X from 5′ to 3′ are abasic and the third instances of X is G. In some aspects of Formula (Va), the first and third instances of X from 5′ to 3′ are abasic and the second instances of X is G. In some aspects of Formula (Va), the second and third instances of X from 5′ to 3′ are abasic and the first instances of X is G.

In some embodiments, including, for example, some embodiments of Formula (V), the antisense oligomer is according to Formula (Vb):

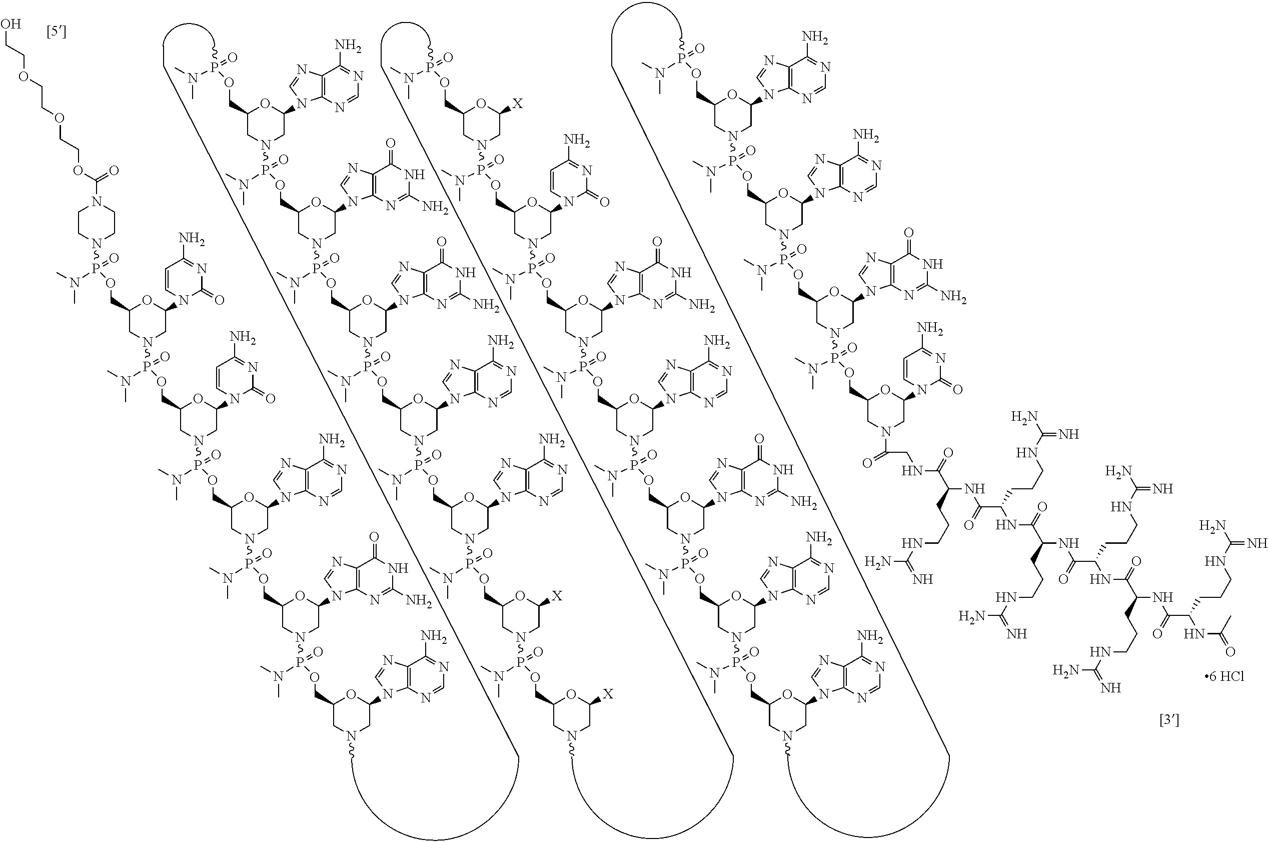

wherein each X is independently selected from guanine (G) or is abasic (B), wherein at least one X is B.

In an embodiment, B is H.

In some aspects of Formula (Vb), one instance of X is abasic and the other instances of X are each G. In certain aspects, two instances of X are abasic and one instance is G. In some aspects of Formula (Vb), the first instance of X from 5′ to 3′ is abasic and the other two instances of X are G. In some aspects of Formula (Vb), the second instance of X from 5′ to 3′ is abasic and the first and third instance of X are G. In certain aspects of Formula of (Vb), the third instance of X from 5′ to 3′ is abasic and the first and second instance of X are G.

In some aspects of Formula (Vb), two instances of X are abasic and the other instance of X is G. In some aspects of Formula (Vb), the first and second instances of X from 5′ to 3′ are abasic and the third instances of X is G. In some aspects of Formula of (Vb), the first and third instances of X from 5′ to 3′ are abasic and the second instances of X is G. In some aspects of Formula of (Vb), the second and third instances of X from 5′ to 3′ are abasic and the first instances of X is G.

In some embodiments, including, for example, some embodiments of Formula (V), the antisense oligomer is according to Formula (VII):

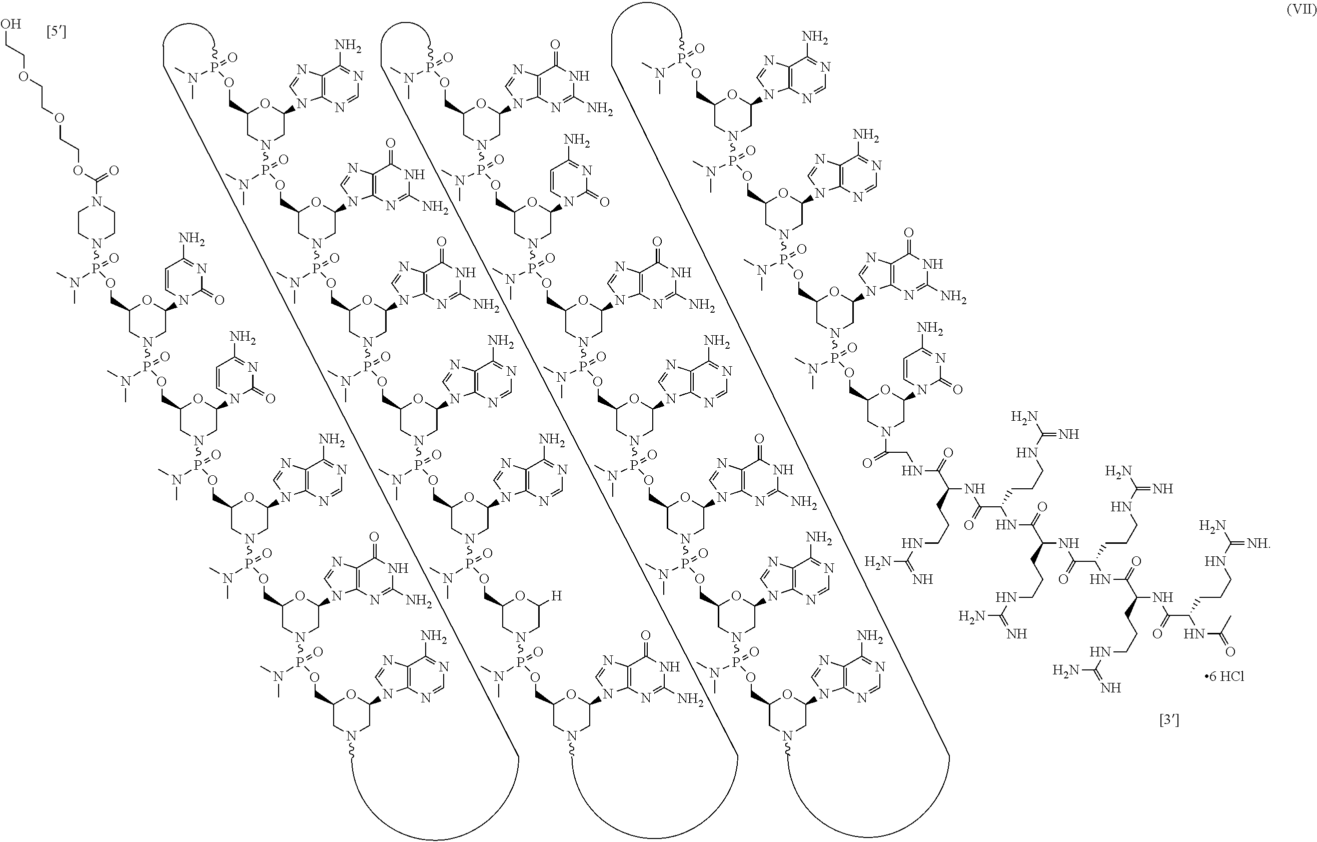

In some embodiments, including, for example, some embodiments of Formula (V), the antisense oligomer is according to Formula (VIII):

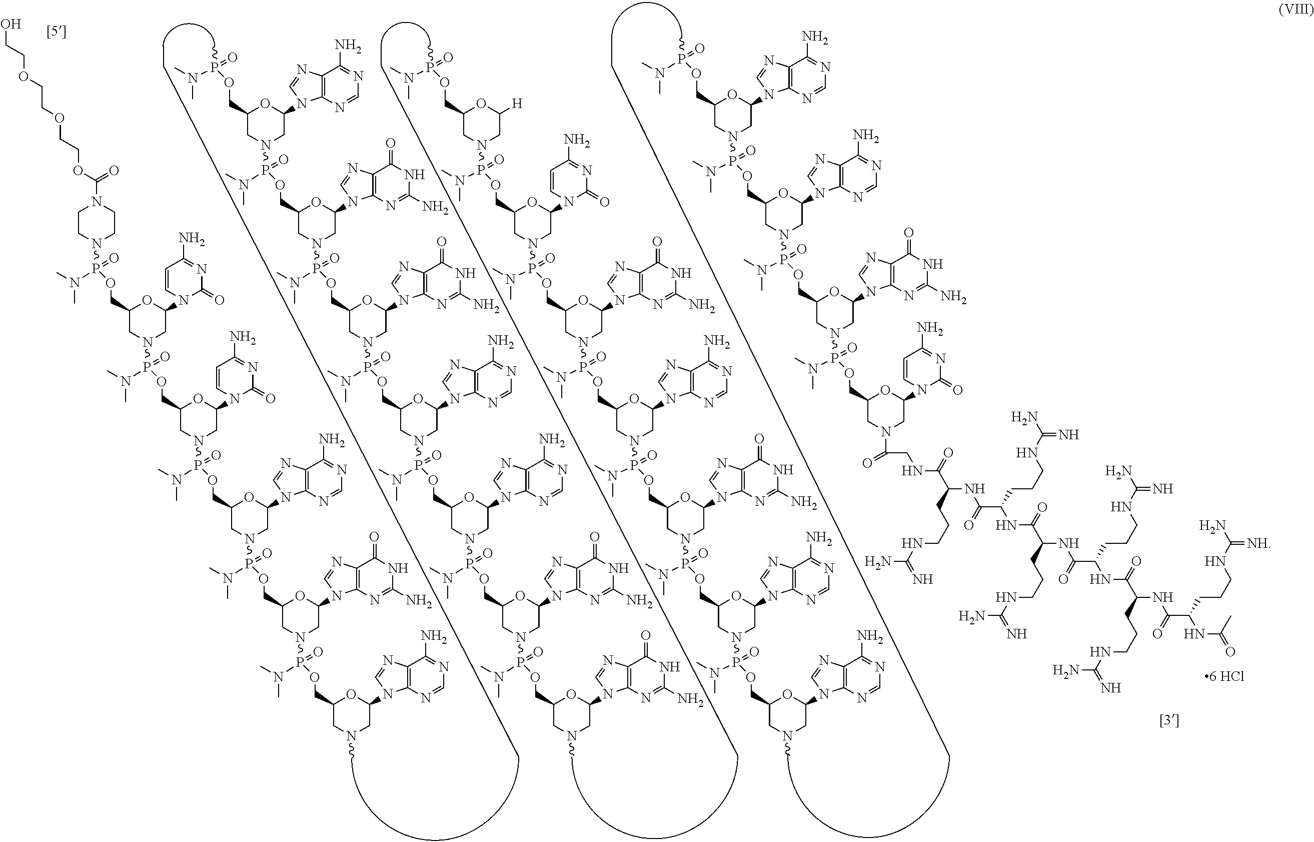

In some embodiments, including, for example, some embodiments of Formula (V), the antisense oligomer is according to Formula (Vc):

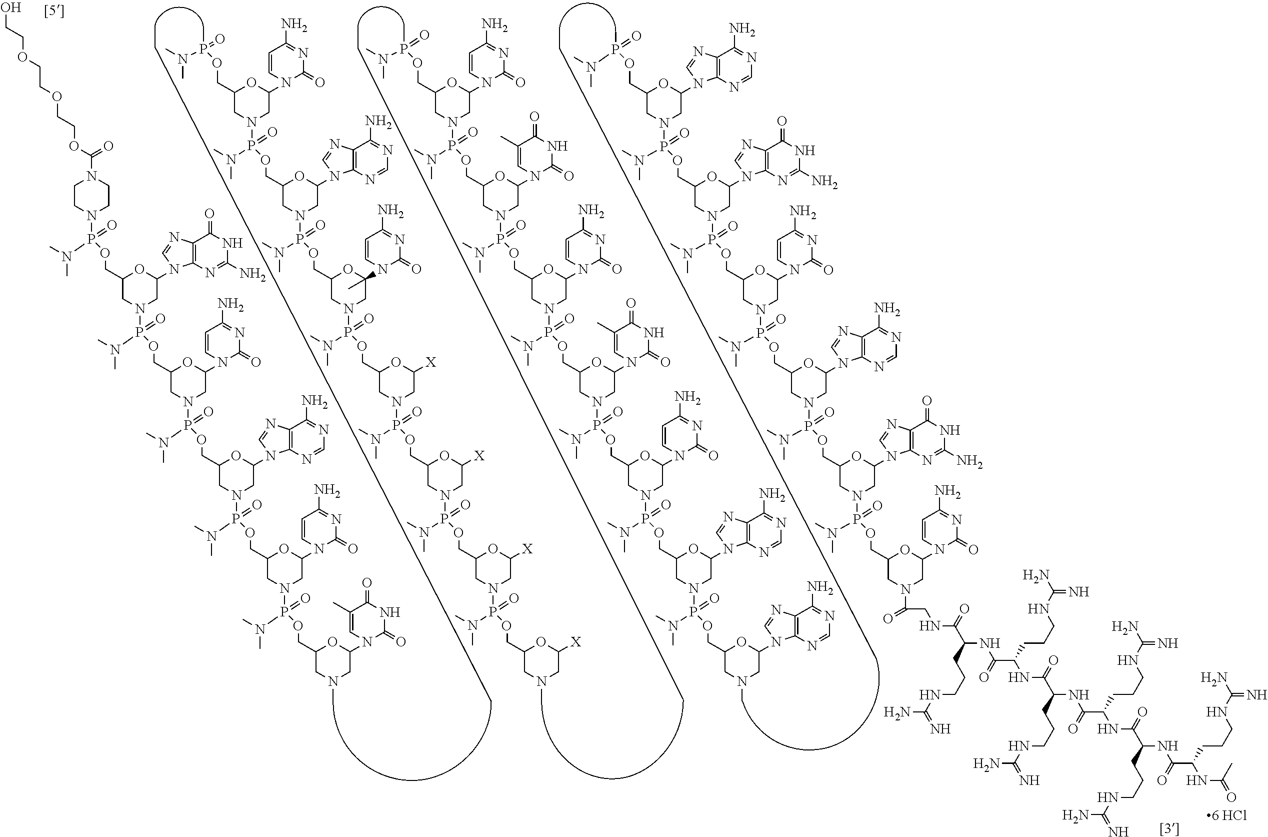

wherein each X is independently selected from guanine (G) or is abasic (B), wherein at least one X is B.

In an embodiment, B is H.

In some aspects of Formula (Vc), one instance of X is abasic and the other instances of X are each G. In certain aspects of Formula (Vc), two instances of X are abasic and one instance is G. In some aspects of Formula (Vc), the first instance of X from 5 to 3 is abasic and the other two instances of X are G. In some aspects of Formula (Vc), the second instance of X from 5′ to 3′ is abasic and the first and third instance of X are G. In certain aspects of Formula (Vc), the third instance of X from 5′ to 3′ is abasic and the first and second instance of X s G.

In some aspects of Formula (Vc), two instances of X are abasic and the other instance of X is G. In some aspects of Formula (V/c), the first and second instances of X from 5′ to 3′ are abasic and the third instances of X is G. In some aspects of Formula (Vc), the first and third instances of X from 5′ to 3′ are abasic and the second instances of X is G. In some aspects of Formula (N/c), the second and third instances of X from 5′ to 3′ are abasic and the first instances of X is G.

In some embodiments, including, for example, some embodiments of Formula (V), the antisense oligomer is according to Formula (Vd):

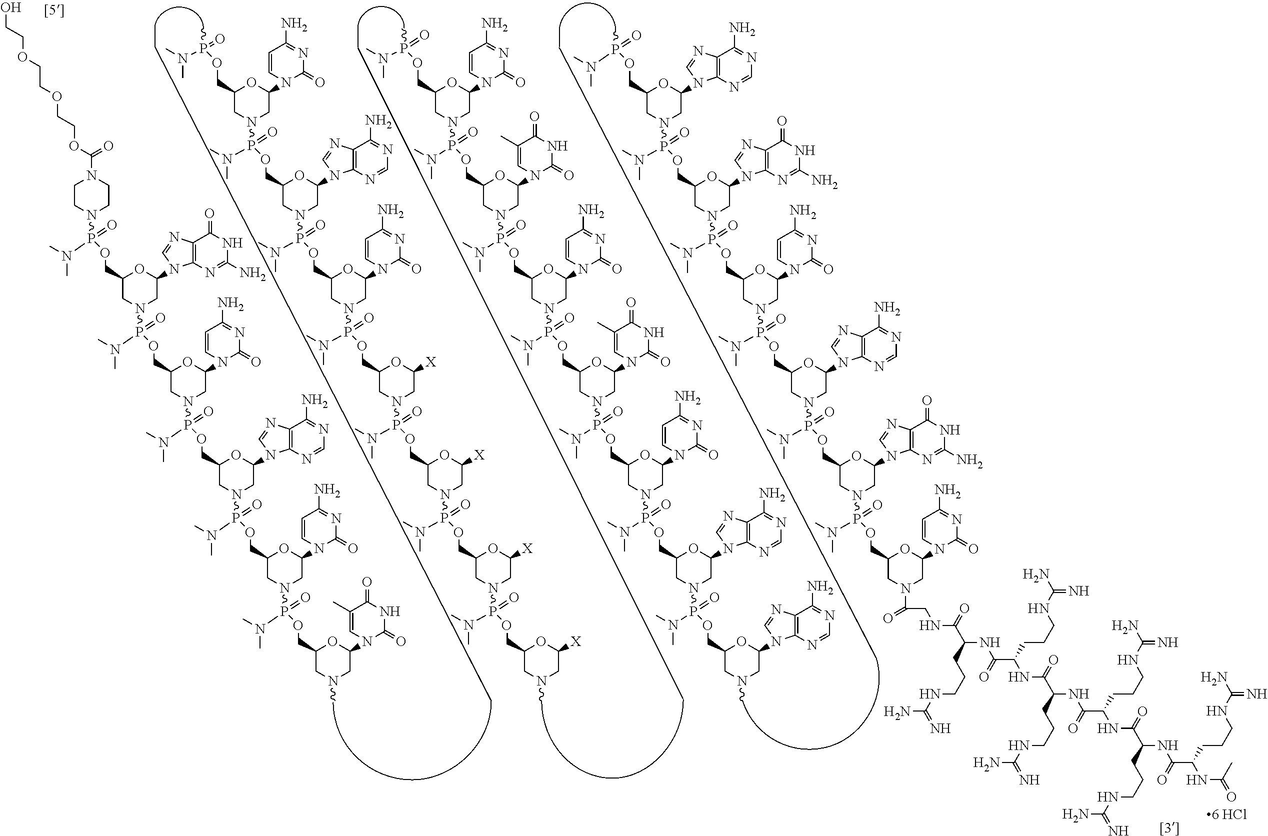

wherein each X is independently selected from guanine (G) or is abasic (B), wherein at least one X is B.

In an embodiment, B is H.

In some aspects of Formula (Vd), one instance of X is abasic and the other instances of X are each G. In certain aspects of Formula (Vd), two instances of X are abasic and one instance is G. In some aspects of Formula (Vd), the first instance of X from 5′ to 3′ is abasic and the other two instances of X are G. In some aspects of Formula (Vd), the second instance of X from 5′ to 3′ is abasic and the first and third instance of X are G. In certain aspects of Formula (Vd), the third instance of X from 5′ to 3′ is abasic and the first and second instance of X are G.

In some aspects of Formula (Vd), two instances of X are abasic and the other instance of X is G. In some aspects of Formula (Vd), the first and second instances of X from 5′ to 3′ are abasic and the third instances of X is G. In some aspects of Formula (Vd), the first and third instances of X from 5′ to 3′ are abasic and the second instances of X is G. In some aspects of Formula (Vd), the second and third instances of X from 5′ to 3′ are abasic and the first instances of X is G.

### IV. Target Sequences and Target Regions

In some embodiments for antisense applications, the oligomer can be 100% complementary to the nucleic acid target sequence (excluding at least one abasic subunit), or it may include mismatches, e.g., to accommodate variants, as long as a heteroduplex formed between the oligomer and nucleic acid target sequence is sufficiently stable to withstand the action of cellular nucleases and other modes of degradation which may occur in vivo. Mismatches, if present, are less destabilizing toward the end regions of the hybrid duplex than in the middle. The number of mismatches allowed will depend on the length of the oligomer, the percentage of G:C base pairs in the duplex, and the position of the mismatch(es) in the duplex, according to well-understood principles of duplex stability. Although such an antisense oligomer is not necessarily 100% complementary to the nucleic acid target sequence, it is effective to stably and specifically bind to the target sequence, such that a biological activity of the nucleic acid target, e.g., expression of the encoded protein(s), is modulated.

The stability of the duplex formed between an oligomer and the target sequence is a function of the binding Tₘ and the susceptibility of the duplex to cellular enzymatic cleavage. The Tₘ of an antisense compound with respect to complementary-sequence RNA may be measured by conventional methods, such as those described by Hames et al., Nucleic Acid Hybridization, IRL Press, 1985, pp. 107-108 or as described in Miyada C G. and Wallace R B (1987) Oligonucleotide hybridization techniques, Methods Enzymol. Vol. 154 pp. 94-107.

In some embodiments, each antisense oligomer has a binding Tₘ, with respect to a complementary-sequence RNA, of greater than body temperature or in other embodiments greater than 50° C. In other embodiments, Tₘ's are in the range 60-80° C. or greater. According to well-known principles, the Tₘ of an oligomer compound, with respect to a complementary-based RNA hybrid, can be increased by increasing the ratio of C:G paired bases in the duplex, and/or by increasing the length (in base pairs) of the heteroduplex. At the same time, for purposes of optimizing cellular uptake, it may be advantageous to limit the size of the oligomer. For this reason, compounds that show high Tₘ (50° C. or greater) at a length of 20 bases or less are generally preferred over those requiring greater than 20 bases for high Tₘ values. For some applications, longer oligomers, for example longer than 20 bases, may have certain advantages.

The targeting sequence bases may be normal DNA bases or analogues thereof, e.g., uracil and inosine that are capable of Watson-Crick base pairing to target-sequence RNA bases.

An antisense oligomer can be designed to block or inhibit or modulate translation of mRNA or to inhibit or modulate pre-mRNA splice processing, or induce degradation of targeted mRNAs, and may be said to be “directed to” or “targeted against” a target sequence with which it hybridizes. In certain embodiments, the target sequence includes a region including a 3′ or 5′ splice site of a pre-processed mRNA, a branch point, or other sequence involved in the regulation of splicing. The target sequence may be within an exon or within an intron or spanning an intron/exon junction.

An antisense oligomer having a sufficient sequence complementarity to a target RNA sequence to modulate splicing of the target RNA means that the antisense agent has a sequence sufficient to trigger the masking of a binding site for a native protein that would otherwise modulate splicing and/or alters the three-dimensional structure of the targeted RNA. Likewise, an oligomer reagent having a sufficient sequence complementary to a target RNA sequence to modulate splicing of the target RNA means that the oligomer reagent has a sequence sufficient to trigger the masking of a binding site for a native protein that would otherwise modulate splicing and/or alters the three-dimensional structure of the targeted RNA.

In certain embodiments, the antisense oligomer has sufficient length and complementarity to a sequence in intron 1 of the human GAA pre-mRNA. The intron 1 (SEQ ID NO:1) sequences for human the GAA gene are shown in Table 2 below (The highlighted T/G near the 3′ end of SEQ ID NO:1 is the IVS1-13T>G mutation described above; the nucleotide at this position is either T or G).

TABLE 2
Target Region for GAA-targeted oligomers

| | | SEQ |
|---|---|---|
| Name | Sequence(5′-3′) | ID NO |
| GAA-IVS1 | GTGAGACACCTGACGTCTGCCCCGCGCTGCCGGCGGTAACATCCCAGAA | 1 |
| | GCGGGTTTGAACGTGCCTAGCCGTGCCCCCAGCCTCTTCCCCTGAGCGG | |
| | AGCTTGAGCCCCAGACCTCTAGTCCTCCCGGTCTTTATCTGAGTTCAGCT | |
| | TAGAGATGAACGGGGAGCCGCCCTCCTGTGCTGGGCTTGGGGCTGGAG | |
| | GCTGCATCTTCCCGTTTCTAGGGTTTCCTTTCCCCTTTTGATCGACGCAGT | |
| | GCTCAGTCCTGGCCGGGACCCGAGCCACCTCTCCTGCTCCTGCAGGACG | |
| | CACATGGCTGGGTCTGAATCCCTGGGGTGAGGAGCACCGTGGCCTGAGA | |
| | GGGGGCCCCTGGGCCAGCTCTGAAATCTGAATGTCTCAATCACAAAGAC | |
| | CCCCTTAGGCCAGGCCAGGGGTGACTGTCTCTGGTCTTTGTCCCTGGTT | |
| | GCTGGCACATAGCACCCGAAACCCTTGGAAACCGAGTGATGAGAGAGCC | |
| | TTTTGCTCATGAGGTGACTGATGACCGGGGACACCAGGTGGCTTCAGGA | |
| | TGGAAGCAGATGGCCAGAAAGACCAAGGCCTGATGACGGGTTGGGATGG | |
| | AAAAGGGGTGAGGGGCTGGAGATTGAGTGAATCACCAGTGGCTTAGTCA | |
| | ACCATGCCTGCACAATGGAACCCCGTAAGAAACCACAGGGATCAGAGGG | |
| | CTTCCCGCCGGGTTGTGGAACACACCAAGGCACTGGAGGGTGGTGCGA | |
| | GCAGAGAGCACAGCATCACTGCCCCCACCTCACACCAGGCCCTACGCAT | |
| | CTCTTCCATACGGCTGTCTGAGTTTTATCCTTTGTAATAAACCAGCAACTG | |
| | TAAGAAACGCACTTTCCTGAGTTCTGTGACCCTGAAGAGGGAGTCCTGGG | |
| | AACCTCTGAATTTATAACTAGTTGATCGAAAGTACAAGTGACAACCTGGGA | |
| | TTTGCCATTGGCCTCTGAAGTGAAGGCAGTGTTGTGGGACTGAGCCCTTA | |
| | ACCTGTGGAGTCTGTGCTGACTCCAGGTAGTGTCAAGATTGAATTGAATT | |
| | GTAGGACACCCAGCCGTGTCCAGAAAGTTGCAGAATTGATGGGTGTGAG | |
| | AAAAACCCTACACATTTAATGTCAGAAGTGTGGGTAAAATGTTTCACCCTC | |
| | CAGCCCAGAGAGCCCTAATTTACCAGTGGCCCACGGTGGAACACCACGT | |
| | CCGGCCGGGGGCAGAGCGTTCCCAGCCAAGCCTTCTGTAACATGACATG | |
| | ACAGGTCAGACTCCCTCGGGCCCTGAGTTCACTTCTTCCTGGTATGTGAC | |
| | CAGCTCCCAGTACCAGAGAAGGTTGCACAGTCCTCTGCTCCAAGGAGCTT | |
| | CACTGGCCAGGGGCTGCTTTCTGAAATCCTTGCCTGCCTCTGCTCCAAGG | |
| | CCCGTTCCTCAGAGACGCAGACCCCTCTGATGGCTGACTTTGGTTTGAGG | |
| | ACCTCTCTGCATCCCTCCCCCATGGCCTTGCTCCTAGGACACCTTCTTCC | |
| | TCCTTTCCCTGGGGTCAGACTTGCCTAGGTGCGGTGGCTCTCCCAGCCTT | |
| | CCCCACGCCCTCCCCATGGTGTATTACACACACCAAAGGGACTCCCCTAT | |
| | TGAAATCCATGCATATTGAATCGCATGTGGGTTCCGGCTGCTCCTGGGAG | |
| | GAGCCAGGCTAATAGAATGTTTGCCATAAAATATTAATGTACAGAGAAGC | |
| | GAAACAAAGGTCGTTGGTACTTGTTAACCTTACCAGCAGAATAATGAAAG | |
| | CGAACCCCCATATCTCATCTGCACGCGACATCCTTGTTGTGTCTGTACCC | |
| | GAGGCTCCAGGTGCAGCCACTGTTACAGAGACTGTGTTTCTTCCCCATGT | |
| | ACCTCGGGGGCCGGGAGGGGTTCTGATCTGCAAAGTCGCCAGAGGTTAA | |
| | GTCCTTTCTCTCTTGTGGCTTTGCCACCCCTGGAGTGTCACCCTCAGCTG | |
| | CGGTGCCCAGGATTCCCCACTGTGGTATGTCCGTGCACCAGTCAATAGG | |
| | AAAGGGAGCAAGGAAAGGTACTGGGTCCCCCTAAGGACATACGAGTTGC | |
| | CAGAATCACTTCCGCTGACACCCAGTGGACCAAGCCGCACCTTTATGCAG | |
| | AAGTGGGGCTCCCAGCCAGGCGTGGTCACTCCTGAAATCCCAGCACTTC | |
| | GGAAGGCCAAGGGGGGTGGATCACTTGAGCTCAGGAGTTCGAGACCAG | |
| | CCTGGGTAACATGGCAAAATCCCGTCTCTACAAAAATACAGAAAATTAGCT | |
| | GGGTGCGGTGGTGTGTGCCTACAGTCCCAGCTACTCAGGAGGCTGAAGT | |
| | GGGAGGATTGCTTGAGTCTGGGAGGTGGAGGTTGCAGTGAGCCAGGATC | |
| | TCACCACAGCACTCTGGCCCAGGCGACAGCTGTTTGGCCTGTTTCAAGTG | |
| | TCTACCTGCCTTGCTGGTCTTCCTGGGGACATTCTAAGCGTGTTTGATTTG | |
| | TAACATTTTAGCAGACTGTGCAAGTGCTCTGCACTCCCCTGCTGGAGCTT | |
| | TTCTCGCCCTTCCTTCTGGCCCTCTCCCCAGTCTAGACAGCAGGGCAACA | |
| | CCCACCCTGGCCACCTTACCCCACCTGCCTGGGTGCTGCAGTGCCAGCC | |
| | GCGGTTGATGTCTCAGAGCTGCTTTGAGAGCCCCGTGAGTGCCGCCCCT | |
| | CCCGCCTCCCTGCTGAGCCCGCTTT/GCTTCTCCCGCAG | |
| GAA-IVS1 | GCTTTTCTCGCCCTTCCTTCTGG | 2 |
| (−189-167) | | |
| GAA-IVS1 | GATGTCTCAGAGCTGCTTTGAGAGCCCCGTGAGTGCCGCCCCTCCCGCC | 3 |
| (−80-24) | TCCCTGC | |

In certain embodiments, the degree of complementarity between the target sequence and antisense targeting sequence is sufficient to form a stable duplex. The region of complementarity of the antisense oligomers, excluding the abasic units, with the target RNA sequence may be as short as 8-11 bases, but can be 12-15 bases or more, e.g., 10-40 bases, 12-30 bases, 12-25 bases, 15-25 bases, 12-20 bases, or 15-20 bases, including all integers in between these ranges. An antisense oligomer of about 14-15 bases is generally long enough to have a unique complementary sequence. In certain embodiments, a minimum length of complementary bases may be required to achieve the requisite binding Tm, as discussed herein.

In certain embodiments, oligomers as long as 40 bases may be suitable, where at least a minimum number of bases, e.g., 10-12 bases, are complementary to the target sequence. In some embodiments, facilitated or active uptake in cells is optimized at oligomer lengths of less than about 30 bases. For PMO oligomers, described further herein, an optimum balance of binding stability and uptake generally occurs at lengths of 18-25 bases. Included in the disclosure are antisense oligomers (e.g., PMOs, PMO-X, PNAs, LNAs, 2′-OMe) that consist of about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 bases, in which at least about 6, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 contiguous or non-contiguous bases that are complementary to the desired target sequences.

In certain embodiments, antisense oligomers may be 100% complementary to the target sequence (excluding at least one abasic nucleotide), or may include mismatches, e.g., to accommodate variants, as long as a heteroduplex formed between the oligomer and target sequence is sufficiently stable to withstand the action of cellular nucleases and other modes of degradation which may occur in vivo. Hence, certain oligomers may have substantial complementarity, meaning, about or at least about 70% sequence complementarity, e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence complementarity, between the oligomer (excluding at least one abasic nucleotide) and the target sequence. Oligomer backbones that are less susceptible to cleavage by nucleases are discussed herein. Mismatches, if present, are typically less destabilizing toward the end regions of the hybrid duplex than in the middle. The number of mismatches allowed will depend on the length of the oligomer, the percentage of G:C base pairs in the duplex, and the position of the mismatch(es) in the duplex, according to well-understood principles of duplex stability. Although such an antisense oligomer is not necessarily 100% complementary to the target sequence, it is effective to stably and specifically bind to the target sequence, such that splicing of the target pre-RNA is modulated. The stability of the duplex formed between an oligomer and a target sequence is a function of the binding Tm and the susceptibility of the duplex to cellular enzymatic cleavage. The Tm of an oligomer with respect to complementary-sequence RNA may be measured by conventional methods, such as those described by Hames et al., Nucleic Acid Hybridization, IRL Press, 1985, pp. 107-108 or as described in Miyada C. G. and Wallace R. B., 1987, Oligomer Hybridization Techniques, Methods Enzymol. Vol. 154 pp. 94-107. In certain embodiments, antisense oligomers may have a binding Tm, with respect to a complementary-sequence RNA, of greater than body temperature and preferably greater than about 45° C. or 50° C. Tm's in the range 60-80° C. or greater are also included. According to well-known principles, the Tm of an oligomer, with respect to a complementary-based RNA hybrid, can be increased by increasing the ratio of C:G paired bases in the duplex, and/or by increasing the length (in base pairs) of the heteroduplex. At the same time, for purposes of optimizing cellular uptake, it may be advantageous to limit the size of the oligomer. For this reason, compounds that show high Tm (45-50° C. or greater) at a length of 25 bases or less are generally preferred over those requiring greater than 25 bases for high Tm values.

In certain embodiments, antisense targeting sequences are designed to hybridize to a region of one or more of the target sequences listed in Table 2. Selected antisense targeting sequences can be made shorter, e.g., about 12 bases, or longer, e.g., about 40 bases, and include a small number of mismatches, as long as the sequence is sufficiently complementary to effect splice modulation upon hybridization to the target sequence, and optionally forms with the RNA a heteroduplex having a Tm of 45° C. or greater.

### V. Cell-Penetrating Peptides (CPPs)

Arginine-rich cell-penetrating peptides (CPP) discussed herein, e.g., within the scope of substituent J, can be effective in enhancing penetration of antisense oligomers into a cell and to cause exon skipping in different muscle groups in animal models.

Exemplary arginine-rich peptides are given below in Table 3.

TABLE 3
Arginine-Rich Peptide Transporters (J)

| Name (Designation) | Sequence |
|---|---|
| rTAT | RRRQRRKKR-L |
| Tat | RKKRRQRRR-L |
| R₉F₂ | RRRRRRRRRFF-L |
| R₅F₂R₄ | RRRRRFFRRRR-L |
| R₄ | RRRR-L |
| R₅ | RRRRR-L |
| R₆ | RRRRRR-L |
| R₇ | RRRRRRR-L |
| R₈ | RRRRRRRR-L |
| R₉ | RRRRRRRRR-L |
| (RXR)₄ | RXRRXRRXRRXR-L |
| (RXR)₅ | RXRRXRRXRRXRRXR-L |
| (RXRRβR)₂ | RXRRβRRXRRβR-L |
| (RAR)₄F₂ | RARRARRARRARFFC-L |
| (RGR)₄F₂ | RGRRGRRGRRGRFFC-L |

^{a}Sequences assigned to SEQ ID NOs do not include the linkage portion L. α and β refer to 6-aminohexanoic acid and beta-alanine, respectively.

In another aspect, exemplary cell-penetrating peptides within the scope of substituent J are provided in Table 4. The point of connection to substituent L is as shown in the Table.

TABLE 4
Exemplary Carrier Peptide Sequences

| Name | Sequence (Amino to Carboxy Terminus) |
|---|---|
| (RFF)₃; CP0407 | RFFRFFRFF-L |
| RTR | RTRTRFLRRT-L |
| RFFR | RFFRFFRFFR-L |
| KTR | KTRTKFLKKT-L |
| KFF | KFFKFFKFF-L |
| KFFK | KFFKFFKFFK-L |
| (RFF)₂ | RFFRFF-L |
| (RFF)₂R | RFFRFFR-L |
| Rα | RααRααR-L |
| (RαR)₄; P007 | RαRRαRRαRRαR-L |
| Tat₄₇_58 | YGRKKRRQRRR-L |
| Tat₄₈_58 | GRKKRRQRRR-L |
| Tat₄₉_58 | RKKRRQRRR-L |
| Penetratin | RQIKIWFQNRRMKWKKGG-L |
| Transportan | GWTLNSAGYLLGKINLKALAALAKKIL-L |
| 2αHph-1 | YARVRRRGPRGYARVRRRGPRR-L |
| Hph-1 | YARVRRRGPRR-L |
| Sim-2 | AK AARQAAR-L |
| HSVI VP22 | DAATATRGRSAASRPTERPRAPARSASRPRRPVE-L |
| Pep-1 | KETWWETWWTEWSQPKKKRKV-L |
| Pep-2 | KETWFETWFTEWSQPKKKRKV-L |
| ANTP | RQIKIWFQNRRMKWKK-L |
| R₆Pen | RRRRRR-RQIKIWFQNRRMKWKKGG-L |
| rTat | RRRQRRKKRC-L |
| pTat | CYGRKKRRQRRR-L |
| R₉F₂ | RRRRRRRRRFFC-L |
| R₉CF₂ | RRRRRRRRRCFF-L |
| R₈CF₂R | RRRRRRRRCFFR-L |
| R₆CF₂R₃ | RRRRRRCFFRRR-L |
| R₅FCFR₄ | RRRRRFCFRRRR-L |
| R₅F₂R₄ | RRRRRFFRRRR-L |
| R₄CF₂R₅ | RRRRCFFRRRRR-L |
| R₂CF₂R₇ | RRCFFRRRRRRR-L |
| CF₂R₉ | CFFRRRRRRRRR-L |
| CR₉F₂ | CRRRRRRRRRFF-L |
| F₂R₉ | FFRRRRRRRRR-L |
| R₅F₂CF₂R₄ | RRRRRFFCFFRRRR-L |
| R₉I₂ | RRRRRRRRRII-L |
| R₈F₃ | RRRRRRRRFFF-L |
| R₉F₄ | RRRRRRRRRFFFF-L |
| RₛF₂ | RRRRRRRRFF-L |
| R₆F₂ | RRRRRRFF-L |
| R₅F₂ | RRRRRFF-L |
| (RRα)₃RR | RRαRRαRRαRR-L |
| (RαR)₄ | RαRRαRRαRRαR-L |
| (αRR)₄ | αRRαRRαRRαRR-L |
| (Rα)₅RR | RαRαRαRαRαR-L |
| (RαR)₃ | RαRRαRRαR-L |
| (RαR)₂R | RαRRαRR-L |
| (RαR)₂ | RαRRαR-L |
| (RKα)₃RK | RKαRKαRKαRK-L |
| (RHα)₃RH | RHαRHαRHαRH-L |
| R₈CF₂R | RRRRRRRRCFFR-L |
| (RRα)₃RR | RRαRRαRRαRR-L |
| (RαR)₄; P007 | RαRRαRRαRRαR-L |
| (aRR)₄ | αRRαRRαRRαRR-L |
| (Rα)₅R | RαRαRαRαRαR-L |
| (Rα)₇R | RαRαRαRαRαRαR-L |
| (RαR)₅ | RαRRαRRαRRαRRαR-L |
| (RαRRβR)₂; B | RαRRβRRαRRβR-L |
| (RαR)₃RβR | RαRRαRRαRRβR-L |
| (Rβ)₅RαRβR | RβRβRβRβRβRαRβR-L |
| RβRβRβRαRβRβRβR | RβRβRβRαRβRβRβR-L |
| α(Rβ)₃Rα(Rβ)₃R-α | αRβRβRβRαRβRβRβR-L |
| (RβRα)₄ | RβRαRβRαRβRαRβR-L |
| (Rβ)₄(Rα)₃R | RβRβRβRβRαRαRαR-L |
| Rα(Rβ)₂Rα(Rβ)₃R | RαRβRβRαRβRβRβR-L |
| (Rβ)₇R | RβRβRβRβRβRβRβR-L |
| R₄ | tg-RRRR-L |
| R₅ | tg-RRRRR-L |
| R₆ | tg-RRRRRR-L |
| R₇ | tg-RRRRRRR-L |
| R₈ | tg-RRRRRRRR-L |
| R₅GR₄ | tg-RRRRRGRRRR-L |
| R₅F₂R₄ | tg-RRRRRFFRRRR-L |
| Tat | tg-RKKRRQRRR-L |
| rTat | tg-RRRQRRKKR-L |
| | RαRRαR-L |
| | RβRRβR-L |
| | RαRRβR-L |
| | RβRRαR-L |
| | RαRY^{b}RαR-L |
| | RβRY^{b}RβR-L |
| | RαRY^{b}RβR-L |
| | RβRY^{b}RAR-L |
| | RαRILFQYRαR-L |
| | RβRILFQYRβR-L |
| | RαRILFQYRβR-L |
| | RβRILFQYRαR-L |
| | RαRRαRRαR-L |
| | RβRRβRRβR-L |
| | RαRRβRRαR-L |
| | RαRRβRRβR-L |
| | RαRRαRRβR-L |
| | RβRRαRRβR-L |
| | RβRRαRRαR-L |
| | RβRRβRRαR-L |
| | RαRY^{b}RαRRαR-L |
| | RαRRαRY^{b}RαR-L |
| | RαRILFQYRαRRαR-L |
| | RαRRαRILFQYRαR-L |
| | RαRY^{b}RαRY^{b}RαR-L |
| | RαRILFQYRαRILFQYRαR-L |
| | RαRILFQYRαRY^{b}RαR-L |
| | RαRY^{b}RαRILFQYRαR-L |
| | RβRY^{b}RβRRβR-L |
| | RβRRβRY^{b}RβR-L |
| | RβRILFQYRβRRβR-L |
| | RβRRβRILFQYRβR-L |
| | RβRYRβRY^{b}RβR-L |
| | RβRILFQYRβRILFQYRβR-L |
| | RβRY^{b}RβRILFQYRβR-L |
| | RβRILFQYRβRY^{b}RβR-L |
| | RαRY^{b}RβRRαR-L |
| | RαRRβRY^{b}RαR-L |
| | RαRILFQYRβRRαR-L |
| | RαRRβRILFQYRαR-L |
| | RαRY^{b}RβRY^{b}RαR-L |
| | RαRILFQYRβRILFQYRαR-L |
| | RαRY^{b}RβRILFQYRαR-L |
| | RαRILFQYRβRY RαR-L |
| | RαRY^{b}RβRRβR-L |
| | RαRRβRY^{b}RβR-L |
| | RαRILFQYRβRRβR-L |
| | RαRRβRILFQYRβR-L |
| | RαRY^{b}RβRY^{b}RβR-L |
| | RαRILFQYRβRILFQYRβR-L |
| | RαRY^{b}RβRILFQYRβR-L |
| | RαRILFQYRβRYRβR-L |
| | RαRY^{b}RαRRβR-L |
| | RαRRαRY^{b}RβR-L |
| | RαRILFQYRαRRβR-L |
| | RαRRαRILFQYRβR-L |
| | RαRY^{b}RαRY^{b}RβR-L |
| | RαRILFQYRαRILFQYRβR-L |
| | RαRY^{b}RαRILFQYRβR-L |
| | RαRILFQYRαRY^{b}RβR-L |
| | RβRY^{b}RαRRβR-L |
| | RβRRαRY^{b}RβR-L |
| | RβRILFQYRαRRβR-L |
| | RβRRαRILFQYRβR-L |
| | RβRY^{b}RαRY^{b}RβR-L |
| | RβRILFQYRαRILFQYRβR-L |
| | RβRY^{b}RαRILFQYRβR-L |
| | RβRILFQYRαRY^{b}RβR-L |
| | RβRY^{b}RαRRαR-L |
| | RβRRαRY^{b}RαR-L |
| | RβRILFQYRαRRαR-L |
| | RβRRαRILFQYRαR-L |
| | RβRY^{b}RαRY^{b}RαR-L |
| | RβRILFQYRαRILFQYRαR-L |
| | RβRY^{b}RαRILFQYRαR-L |
| | RβRILFQYRαRY^{b}RαR-L |
| | RβRY^{b}RβRRαR-L |
| | RβRRβRY^{b}RAR-L |
| | RβRILFQYRβRRαR-L |
| | RβRRβRILFQYRαR-L |
| | RβRYDRβRYDRαR-L |
| | RβRILFQYRβRILFQYRαR-L |
| | RβRY^{b}RβRILFQYRαR-L |
| | RβRILFQYRβRY^{b}RαR-L |
| | RαRRαRRαRRαR-L |
| | RαRRβRRαRILFQYRαRβRαR-L |
| | RαRRβRRαRRβR-L |
| | YGRKKRRQRRRP-L |
| | RαRRαRRαRRαRαβASSLNIAαC-L |
| | RαRRβRRαRILFQYRαRβRαRβASSLNIAαC-L |
| | RαRRβRRαRASSLNIARαRβRαRβC-L |
| | RαRRβRRαRRβRαβASSLNIA-L |
| | THRPPMWSPVWP-L |
| | HRPPMWSPVWP-L |
| | THRPPMWSPV-L |
| | THRPPMWSP-L |
| | THRPPMWSPVFP-L |
| | THRPPMWSPVYP-L |
| | THRPPMWSPAWP-L |
| | THRPPMWSPLWP-L |
| | THRPPMWSPIWP-L |
| | THRPPMWTPVVWP-L |
| | THRPPMFSPVWP-L |
| | THRPPMWS-L |
| | HRPPMWSPVW-L |
| | THRPPMYSPVWP-L |
| | THRPPnleWSPVWP-L (nle = norleucine) |
| | THKPPMWSPVWP-L |
| | SHRPPMWSPVWP-L |
| | STFTHPR-L |
| | YDIDNRR-L |
| | AYKPVGR-L |
| | HAIYPRH-L |
| | HTPNSTH-L |
| | ASSPVHR-L |
| | SSLPLRK-L |
| | KKRS-L |
| | KRSK-L |
| | KKRSK-L |
| | KSRK-L |
| | SRKR-L |
| | RKRK-L |
| | KSRKR-L |
| | QHPPWRV-L |
| | THPPTTH-L |
| | YKHTPTT-L |
| | QGMHRGT-L |
| | SRKRK-L |
| | KSRKRK-L |
| | PKKKRKV-L |
| | GKKRSKV-L |
| | KSRKRKL-L |
| | HSPSKIP-L |
| | HMATFHY-L |
| | AQPNKFK-L |
| | NLTRLHT-L |
| | KKKR-L |
| | KKRK-L |
| | KKKRK-L |
| | RRRRRRQIKIWFQNRRMKWKKGGC-L |
| | RRRRRRRQIKIWFQNRRMKWKKGGC-L |
| | RQIKIWFQNRRMKWKKGGC-L |
| | RRRRRRRQIKIWFQNRRMKWKKC-L |
| | RαRRαRRαRRQIKIWFQNRRMKWKKGGC-L |
| | RRRRRRRQIKILFQNRαRαRαRαC-L |
| | RαRRαRRαRRαRC-L |
| | RαRRαRRαRRαRαC-L |
| | RαRRαRRαRIKILFQNRRMKWKKGGC-L |
| | RαRRαRRαRIKILFQNRRMKWKKC-L |
| | RαRRαRRαRIKILFQNRMKWKKC-L |
| | RαRRαRRαRIKILFQNαRMKWKKC-L |
| | RαRRαRRαRIKILFQNHRMKWKKC-L |
| | RαRRαRRαRIKILFQNαRMKWKKC-L |
| | RαRRαRRαRIKILFQNαRMKWKKC-L |
| | RαRRαRRαRIKILFQNαRMKWKAC-L |
| | RαRRαRRαRIKILFQNαRMKWHKAC-L |
| | RαRRαRRαRIKILFQNαRMKWHRC-L |
| | RαRαRαRαRIKILFQNRRMKWKKC-L |
| | RARARARARIKILFQNRRMKWKKC-L |
| | RαRRαRRαRIαILFQNαRMKWHKAC-L |
| | RαRRαRRαRIHILFQNαRMKWHKAC-L |
| | RαRRαRRαRIRILFQNαRMKWHKAC-L |
| | RαRRαRRαRIαILFQYαRMKWHKAC-L |
| | RαRRαRRαRLYSPLSFQαRMKWHKAC-L |
| | RαRRαRRαRISILFQYαRMKWHKAC-L |
| | RαRRαRRαRILFQYαRMKWHKAC-L |
| | RαRRαRIαILFQYαRMKWHKAC-L |
| | RαRRARRαRIHILFQYαRMKWHKAC-L |
| | RARRαRRARIHILFQYαRMKWHKAC-L |
| | RαRRαRRαRIHILFQYαRMKWHKAC-L |
| | RαRRαRRαRIαILFQNαRMKWHKAC-L |
| | RαRRαRRαRIHILFQNαRMKWHKAC-L |
| | RαRRαRRαRIKILFQNRRMKWHK-L |
| | RαRRαRRαRIKILFQNαRMKWHK-L |
| | RαRRαRRαRIαILFQNRRMKWHK-L |
| | RαRRαRRαRIαILFQNαRMKWHK-L |
| | RαRRαRRαRIHILFQNRRMKWHK-L |
| | RαRRαRRαRIHILFQNαRMKWHK-L |
| | RαRRαRRαRIRILFQNRRMKWHK-L |
| | RαRRαRRαRIRILFQNαRMKWHK-L |
| | RαRRαRRαRIILFQNRRMKWHK-L |
| | RαRRαRRαRIILFQNαRMKWHK-L |
| | RαRRαRRαRKILFQNRRMKWHK-L |
| | RαRRαRRαRKILFQNαRMKWHK-L |
| | RαRRαRRαRαILFQNRRMKWHK-L |
| | RαRRαRRαRαILFQNαRMKWHK-L |
| | RαRRαRRαRHILFQNRRMKWHK-L |
| | RαRRαRRαRHILFQNαRMKWHK-L |
| | RαRRαRRαRRILFQNRRMKWHK-L |
| | RαRRαRRαRRILFQNαRMKWHK-L |
| | RαRRαRRαRILFQNRRMKWHK-L |
| | RαRRαRRαRILFQNαRMKWHK-L |
| | RαRRαRRαRIKILFQYRRMKWHK-L |
| | RαRRαRRαRIKILFQYαRMKWHK-L |
| | RαRRαRRαRIαILFQYRRMKWHK-L |
| | RαRRαRRαRIαILFQYαRMKWHK-L |
| | RαRRαRRαRIHILFQYRRMKWHK-L |
| | RαRRαRRαRIHILFQYαRMKWHK-L |
| | RαRRαRRαRIRILFQYRRMKWHK-L |
| | RαRRαRRαRIRILFQYαRMKWHK-L |
| | RαRRαRRαRIILFQYRRMKWHK-L |
| | RαRRαRRαRIILFQYαRMKWHK-L |
| | RαRRαRRαRKILFQYRRMKWHK-L |
| | RαRRαRRαRKILFQYαRMKWHK-L |
| | RαRRαRRαRαILFQYRRMKWHK-L |
| | RαRRαRRαRαILFQYαRMKWHK-L |
| | RαRRαRRαRHILFQYRRMKWHK-L |
| | RαRRαRRαRHILFQYαRMKWHK-L |
| | RαRRαRRαRRILFQYRRMKWHK-L |
| | RαRRαRRαRRILFQYαRMKWHK-L |
| | RαRRαRRαRILFQYRRMKWHK-L |
| | RαRRαRRαRILFQYαRMKWHK-L |
| | RαRRαRRαR-L |
| | RαRRαRRαRRαR-L |
| | RARRAR-L |
| | RARRARRAR-L |
| | RARRARRARRAR-L |
| | RαRRαRI-L |
| | RαRRARRαR-L |
| | RARRαRRAR-L |
| | RRRRR-L |
| | RRRRRR-L |
| | RRRRRRR-L |
| | RαRRαRRαRRαRC-L |
| | RαRRαRRαRRαRαC-L |
| | RαRRαRRαRIKILFQNRRMKWKKGGC-L |
| | RαRRαRRαRIKILFQNRRMKWKKC-L |
| | RαRRαRRαRIKILFQNRMKWKKC-L |
| | RαRRαRRαRIKILFQNαRMKWKKC-L |
| | RαRRαRRαRIKILFQNHRMKWKKC-L |
| | RαRRαRRαRIKILFQNαRMKWKKC-L |
| | RαRRαRRαRIKILFQNαRMKWKKC-L |
| | RαRRαRRαRIKILFQNαRMKWKAC-L |
| | RαRRαRRαRIKILFQNαRMKWHKAC-L |
| | RαRRαRRαRIKILFQNαRMKWHRC-L |
| | RαRαRαRαRIKILFQNRRMKWKKC-L |
| | RARARARARIKILFQNRRMKWKKC-L |
| | RαRRαRRαRIαILFQNαRMKWHKAC-L |
| | RαRRαRRαRIHILFQNαRMKWHKAC-L |
| | RαRRαRRαRIRILFQNαRMKWHKAC-L |
| | RαRRαRRαRIαILFQYαRMKWHKAC-L |
| | RαRRαRRαRLYSPLSFQαRMKWHKAC-L |
| | RRMKWHK-L |
| | αRMKWHK-L |
| | ααααααααααααααILFQααRMKWHK-L |
| | ααααααααααααααILFQααRMKWHK-L |
| | RRRRRRRQIKILFQNPKKKRKVGGC-L |
| | HHFFRRRRRRRRRFFC-L |
| | HHHHHHRRRRRRRRRFFC-L |
| | HHHHHHFFRRRRRRRRRFFC-L |
| | HHHHHααRRRRRRRRRFFC-L |
| | HHHHHHααFFRRRRRRRRRFFC-L |
| | HHHαRRRRRRRRRFFαHHHC-L |
| | αRMKWHK-L |
| | αRWKWHK-L |
| | RαRARαR-L |
| | RαRαRαR-L |
| | RARαRAR-L |
| | RαRAR-L |
| | ααααααααααααααILFQααHMKWHK-L |
| | ααααααααααααααILFQααRWKWHK-L |
| | ααααααααααααααILFQααHWKWHK-L |
| | ααααααααααααααILFQαRαRARαR-L |
| | ααααααααααααααILFQαRαRαRαR-L |
| | ααααααααααααααILFQαRαRRαR-L |
| | ααααααααααααααILFQαRARαRAR-L |
| | ααααααααααααααILFQαRαRARαR-L |
| | ααααααααααααααILFQαRαRAR-L |
| | ααααααααααααααILIQααRMKWHK-L |
| | ααααααααααααααILIQααHMKWHK-L |
| | ααααααααααααααILIQααRWKWHK-L |
| | ααααααααααααααILIQααHWKWHK-L |
| | ααααααααααααααILIQαRαRARαR-L |
| | ααααααααααααααILIQαRαRαRαR-L |
| | ααααααααααααααILIQαRαRRαR-L |
| | ααααααααααααααILIQαRARαRAR-L |
| | ααααααααααααααILIQαRαRARαR-L |
| | ααααααααααααααILIQαRαRAR-L |
| | ααααααααααααααILFQααHMKWHK-L |
| | ααααααααααααααILFQααRWKWHK-L |
| | ααααααααααααααILFQααHWKWHK-L |
| | ααααααααααααααILFQαRαRARαR-L |
| | ααααααααααααααILFQαRαRαRαR-L |
| | ααααααααααααααILFQαRαRRαR-L |
| | ααααααααααααααILFQαRARαRAR-L |
| | ααααααααααααααILFQαRαRARαR-L |
| | ααααααααααααααILFQαRαRAR-L |
| | ααααααααααααααILIQααRMKWHK-L |
| | ααααααααααααααILIQααHMKWHK-L |
| | ααααααααααααααILIQααRWKWHK-L |
| | ααααααααααααααILIQααHWKWHK-L |
| | ααααααααααααααILIQαRαRARαR-L |
| | ααααααααααααααILIQαRαRαRαR-L |
| | ααααααααααααααILIQαRαRRαR-L |
| | ααααααααααααααILIQαRARαRAR-L |
| | ααααααααααααααILIQαRαRARαR-L |
| | ααααααααααααααILIQαRαRAR-L |
| | RαRRARRαRRARαA-L |
| | RαRRARRαRILFQYαHMKWHKAC-L |
| | RαRRARRαRILFQYαRMKWHKAC-L |
| | RαRRARRαRILFQYαRWKWHKAC-L |
| | RαRRαRRαRRαRC-L |
| | RαRRαRRαRIαILFQNαRMKWHKAC-L |
| | RαRRαRRαRIHILFQNαRMKWHKAC-L |
| | RαRRαRRαRIαILFQYαRMKWHKAC-L |
| | RαRRαRRαRLYSPLSFQαRMKWHKAC-L |
| | RαRRαRRαRILFQYαRMKWHKAC-L |
| | RαRRαRIαILFQYαRMKWHKAC-L |
| | RARRαRRARILFQYαRMKWHKAC-L |
| | RαRRARRαRILFQYαRMKWHKAC-L |
| | RARRαRRARILFQYαRMKWHKAC-L |
| | RαRRARRαRILFQYαRMKWHKAC-L |
| | RαRRARRαRILFQYαHMKWHKAC-L |
| | RαRRARRαRILFQYαRMKWHKAC-L |
| | RαRRARRαRILFQYαRWKWHKAC-L |
| | RαRRARRαRILFQYαHWKWHKAC-L |
| | RαRRARRαRILFQYRαRARαRAC-L |
| | RαRRARRαRILFQYRαRαRαRAC-L |
| | RαRRARRαRILIQYαRMKWHKAC-L |
| | RαRRαRILFQYRαRRαRC-L |
| | RαRRARRαRILFQYRαRARαRAC-L |
| | RαRRARRαRILFQYRαRαRαRAC-L |
| | RαRRARRαRILIQYαRMKWHKAC-L |
| | RαRRαRILFQYRαRRαRCYS-L |
| | RARRαRRARILFQYRARαRARAC-L |
| | RARRαRRARILFQYRαRARαRAC-L |
| | RARRαRRARILFQYRαRRαRAC-L |
| | RARRαRRARILFQYRαRARαAC-L |
| | RαRRARRαRILFQYRαRRαRAC-L |
| | RαRRARRαRILFQYRαRARαAC-L |
| | RαRRARRαRIHILFQNαRMKWHKAC-L |
| | RαRRARRαRRARαAC-L |
| | RαRRARRαRILFQYαHMKWHK-L |
| | RαRRARRαRILFQYαRMKWHK-L |
| | RαRRARRαRILFQYαRWKWHK-L |
| | RαRRARRαRILFQYαRMKWHK-L |
| | RαRRARRαRILFQYRαRARαR-L |
| | RαRRARRαRILFQYRαRαRαR-L |
| | RαRRARRαRILFQYRαRRαR-L |
| | RαRRARRαRILFQYRARαRAR-L |
| | RαRRARRαRILFQYRαRAR-L |
| | RαRRARRαRILIQYαHMKWHK-L |
| | RαRRARRαRILIQYαRMKWHK-L |
| | RαRRARRαRILIQYαRWKWHK-L |
| | RαRRARRαRILIQYαRMKWHK-L |
| | RαRRARRαRILIQYRαRARαR-L |
| | RαRRARRαRILIQYRαRαRαR-L |
| | RαRRARRαRILIQYRαRRαR-L |
| | RαRRARRαRILIQYRARαRAR-L |
| | RαRRARRαRILIQYRαRAR-L |
| | RARRαRRARILFQYαHMKWHK-L |
| | RARRαRRARILFQYαRMKWHK-L |
| | RARRαRRARILFQYαRWKWHK-L |
| | RARRαRRARILFQYαRMKWHK-L |
| | RARRαRRARILFQYRαRARαR-L |
| | RARRαRRARILFQYRαRαRαR-L |
| | RARRαRRARILFQYRαRRαR-L |
| | RARRαRRARILFQYRARαRAR-L |
| | RARRαRRARILFQYRαRAR-L |
| | RARRαRRARILIQYαHMKWHK-L |
| | RARRαRRARILIQYαRMKWHK-L |
| | RARRαRRARILIQYαRWKWHK-L |
| | RARRαRRARILIQYαRMKWHK-L |
| | RARRαRRARILIQYRαRARαR-L |
| | RARRαRRARILIQYRαRαRαR-L |
| | RARRαRRARILIQYRαRRαR-L |
| | RARRαRRARILIQYRARαRAR-L |
| | RARRαRRARILIQYRαRAR-L |
| | RαRRαRILFQYαHMKWHK-L |
| | RαRRαRILFQYαRMKWHK-L |
| | RαRRαRILFQYαRWKWHK-L |
| | RαRRαRILFQYαRMKWHK-L |
| | RαRRαRILFQYRαRARαR-L |
| | RαRRαRILFQYRαRαRαR-L |
| | RαRRαRILFQYRαRRαR-L |
| | RαRRαRILFQYRARαRAR-L |
| | RαRRαRILFQYRαRAR-L |
| | RαRRαRILIQYαHMKWHK-L |
| | RαRRαRILIQYαRMKWHK-L |
| | RαRRαRILIQYαRWKWHK-L |
| | RαRRαRILIQYαRMKWHK-L |
| | RαRRαRILIQYRαRARαR-L |
| | RαRRαRILIQYRαRαRαR-L |
| | RαRRαRILIQYRαRRαR-L |
| | RαRRαRILIQYRARαRAR-L |
| | RαRRαRILIQYRαRAR-L |
| | PRPαααααααααααPRG-L |
| | RRRRRRRR-L |
| | RRMKWKK-L |
| | PKKKRKV-L |
| | CKDEPQRRSARLSAKPAPPKPEPKPKKAPAKK-L |
| | RKKRRQRRR-L |
| | RKKRRQRR-L |
| | RKKRRQR-L |
| | KKRRQRRR-L |
| | KKRRQRRR-L |
| | AKKRRQRRR-L |
| | RAKRRQRRR-L |
| | RKARRQRRR-L |
| | RKKARQRRR-L |

L=as recited in Formula (I) and as described throughout the specification; β=3-alanine; α=6-aminohexanoic acid; tg=unmodified amino terminus, or the amino-terminal capped with an acetyl, benzoyl or stearoyl group (i.e., an acetyl amide, benzoyl amide or stearoyl amide) and Y is NH— (CHR)—C(O)— wherein n is 2 to 7 and each R is independently, at each occurrence, hydrogen or methyl. For simplicity, not all sequences are noted with a terminal td group; however, each of the above sequences may comprise an unmodifed amino terminus or an amino terminus capped with an acetyl, benzoyl, or stearoyl group.

### VI. Pharmaceutical Compositions

The present disclosure also provides for the formulation and delivery of the disclosed antisense oligomers. Accordingly, an aspect of the present disclosure is a pharmaceutical composition comprising antisense oligomers as disclosed herein and a pharmaceutically acceptable carrier.

Effective delivery of the antisense oligomers to the target nucleic acid is an important aspect of treatment. Routes of antisense oligomer delivery include, but are not limited to, various systemic routes, including oral and parenteral routes, e.g., intravenous, subcutaneous, intraperitoneal, and intramuscular, as well as inhalation, transdermal, and topical delivery. The appropriate route may be determined by one of skill in the art, as appropriate to the condition of the subject under treatment. For example, an appropriate route for delivery of an antisense oligomer in the treatment of a viral infection of the skin is topical delivery, while the delivery of an antisense oligomer for the treatment of a viral respiratory infection can be intravenous or by inhalation. The antisense oligomer may also be delivered directly to any particular site of viral infection.

The antisense oligomer can be administered in any convenient vehicle which is physiologically and/or pharmaceutically acceptable. Such a composition can include any of a variety of standard pharmaceutically acceptable carriers employed by those of ordinary skill in the art. Examples include, but are not limited to, saline, phosphate-buffered saline (PBS), water (e.g., sterile water for injection), aqueous ethanol, emulsions such as oil/water emulsions or triglyceride emulsions, tablets, and capsules. The choice of a suitable physiologically acceptable carrier will vary dependent upon the chosen mode of administration.

The instant compounds (e.g., an antisense oligomer) can generally be utilized as the free acid or free base. Alternatively, the instant compounds may be used in the form of acid or base addition salts. Acid addition salts of the free amino compounds may be prepared by methods well known in the art and may be formed from organic and inorganic acids. Suitable organic acids include maleic, fumaric, benzoic, ascorbic, succinic, methanesulfonic, acetic, trifluoroacetic, oxalic, propionic, tartaric, salicylic, citric, gluconic, lactic, mandelic, cinnamic, aspartic, stearic, palmitic, glycolic, glutamic, and benzenesulfonic acids. Suitable inorganic acids include hydrochloric, hydrobromic, sulfuric, phosphoric, and nitric acids. Base addition salts included those salts that form with the carboxylate anion and include salts formed with organic and inorganic cations such as those chosen from the alkali and alkaline earth metals (for example, lithium, sodium, potassium, magnesium, barium and calcium), as well as the ammonium ion and substituted derivatives thereof (for example, dibenzylammonium, benzylammonium, 2-hydroxyethylammonium, and the like). Thus, the term “pharmaceutically acceptable salt” of structure (I) is intended to encompass any and all acceptable salt forms.

In addition, prodrugs are also included within the context of this invention. Prodrugs are any covalently bonded carriers that release a compound of structure (I) in vivo when such a prodrug is administered to a patient. Prodrugs are generally prepared by modifying functional groups in a way such that the modification is cleaved, either by routine manipulation or in vivo, yielding the parent compound. Prodrugs include, for example, compounds of this invention wherein hydroxy, amine, or sulfhydryl groups are bonded to any group that, when administered to a patient, cleaves to form the hydroxy, amine, or sulfhydryl groups. Thus, representative examples of prodrugs include (but are not limited to) acetate, formate, and benzoate derivatives of alcohol and amine functional groups of the compounds of structure (I). Further, in the case of a carboxylic acid (—COOH), esters may be employed, such as methyl esters, ethyl esters, and the like.

### VII. Methods of Making

### Preparation of Oligomers with Basic Nitrogen Internucleoside Linkers

Morpholino subunits, the modified intersubunit linkages, and oligomers comprising the same can be prepared as described, for example, in U.S. Pat. Nos. 5,185,444, and 7,943,762, which are incorporated by reference in their entireties. The morpholino subunits can be prepared according to the following general Reaction Scheme 1.

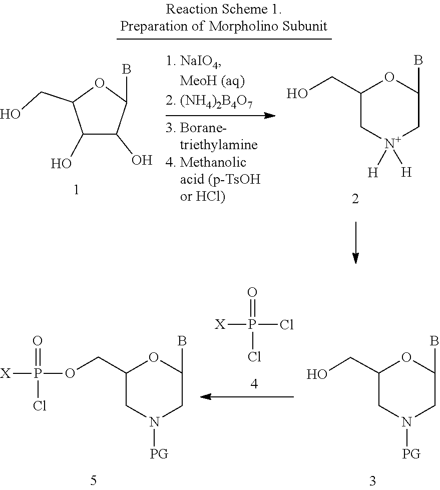

Referring to Reaction Scheme 1, wherein B represents a base-pairing moiety and PG represents a protecting group, the morpholino subunits may be prepared from the corresponding ribonucleoside (1) as shown. The morpholino subunit (2) may be optionally protected by reaction with a suitable protecting group precursor, for example, trityl chloride. The 3′ protecting group is generally removed during solid-state oligomer synthesis as described in more detail below. The base pairing moiety may be suitably protected for sold phase oligomer synthesis. Suitable protecting groups include benzoyl for adenine and cytosine, phenylacetyl for guanine, and pivaloyloxymethyl for hypoxanthine (I). The pivaloyloxymethyl group can be introduced onto the N1 position of the hypoxanthine heterocyclic base. Although an unprotected hypoxanthine subunit, may be employed, yields in activation reactions are far superior when the base is protected. Other suitable protecting groups include those disclosed in U.S. Pat. No. 8,076,476, which is hereby incorporated by reference in its entirety.

Reaction of 3 with the activated phosphorous compound 4 results in morpholino subunits having the desired linkage moiety 5. Compounds of structure 4 can be prepared using any number of methods known to those of skill in the art. For example, such compounds may be prepared by reaction of the corresponding amine and phosphorous oxychloride. In this regard, the amine starting material can be prepared using any method known in the art, for example those methods described in the Examples and in U.S. Pat. No. 7,943,762.

Compounds of structure 5 can be used in solid-phase automated oligomer synthesis for the preparation of oligomers comprising the intersubunit linkages. Such methods are well known in the art. Briefly, a compound of structure 5 may be modified at the 5′ end to contain a linker to a solid support. For example, compound 5 may be linked to a solid support by a linker. Once supported, the protecting group (e.g., trityl) is removed and the free amine is reacted with an activated phosphorous moiety of a second compound of structure 5. This sequence is repeated until the desired length of oligo is obtained. The protecting group in the terminal 5′ end may either be removed or left on if a 5′-modification is desired.

The preparation of modified morpholino subunits and morpholino oligomers are described in more detail in the Examples. The morpholino oligomers containing any number of modified linkages may be prepared using methods described herein, methods known in the art and/or described by reference herein. Also described in the examples are global modifications of morpholino oligomers prepared as previously described (see e.g., PCT publication WO 2008/036127).

Synthesis of PMO, PMO+, PPMO, and PMO-X containing further linkage modifications as described herein was done using methods known in the art and described in pending U.S. Pat. Nos. 8,299,206 and 8,076,476 and PCT publication numbers WO 2009/064471, WO 2011/150408 and WO 2012/150960, which are hereby incorporated by reference in their entirety.

PMO with a 3′ trityl modification are synthesized essentially as described in PCT publication number WO 2009/064471 with the exception that the detritylation step is omitted.

### VIII. Methods of Treatment

Provided herein is a method of increasing the expression of exon 2-containing GAA mRNA and/or protein using the antisense oligomers of the present disclosure for therapeutic purposes (e.g., treating subjects with GSD-II). The method comprises administering to a patient in need thereof a therapeutically effective amount of an antisense oligomer disclosed herein or a pharmaceutical composition thereof. In an embodiment, the disease is Pompe disease. In some embodiments, the antisense oligomer comprising a nucleotide sequence of sufficient length and complementarity to specifically hybridize to a region within the pre-mRNA of the acid alpha-glucosidase (GAA) gene, wherein binding of the antisense oligomer to the region increases the level of exon 2-containing GAA mRNA in a cell and/or tissue of the subject. Exemplary antisense targeting sequences are shown in Tables 6A-6C herein.

Also included are antisense oligomers for use in the preparation of a medicament for the treatment of glycogen storage disease type II (GSD-II; Pompe disease), comprising a nucleotide sequence of sufficient length and complementarity to specifically hybridize to a region within the pre-mRNA of the acid alpha-glucosidase (GAA) gene, wherein binding of the antisense oligomer to the region increases the level of exon 2-containing GAA mRNA.

In some embodiments of the method of treating GSD-II or the medicament for the treatment of GSD-II, the antisense oligomer compound comprises:
an antisense oligomer that is 18-40 subunits in length, comprising a targeting sequence complementary to a target region within intron 1 (SEQ ID NO: 1) of a pre-mRNA of human acid alpha-glucosidase (GAA) gene, wherein:
each subunit of the antisense oligomers comprises a nucleobase or is an abasic subunit;
at least one subunit is an abasic subunit; and
wherein the targeting sequence, except for the abasic subunit or subunits, is at least 80% complementary to the target region. In some embodiments of the method of treating GSD-II or the medicament for the treatment of GSD-II, the antisense oligomer compound comprises:
an antisense oligomer that is 18-40 subunits in length, comprising a targeting sequence complementary to a target region within intron 1 (SEQ ID NO: 1) of a pre-mRNA of human acid alpha-glucosidase (GAA) gene, wherein:
the antisense oligonucleotide comprises a morpholino oligomer;
each subunit of the antisense oligonucleotide comprises a nucleobase or is an abasic subunit, wherein each subunit is taken together in order from the 5′ end of the antisense oligonucleotide to the 3′ end of the antisense oligonucleotide form the targeting sequence;
at least one subunit is an abasic subunit; and
wherein the targeting sequence, except for the abasic subunit or subunits, is at least 80% complementary to the target region.

In some embodiments of the method of treating GSD-II or the medicament for the treatment of GSD-II, the antisense oligomer compound comprises:
a modified antisense oligonucleotide that is 18-40 subunits in length, comprising a targeting sequence complementary to a target region within intron 1 (SEQ ID NO: 1) of a pre-mRNA of human acid alpha glucosidase (GAA) gene, wherein
the antisense oligonucleotide comprises a morpholino oligomer;
the antisense oligonucleotide is covalently linked to the cell-penetrating peptide;
each subunit of the antisense oligonucleotide comprises a nucleobase or is an abasic subunit, wherein each subunit is taken together in order from the 5′ end of the antisense oligonucleotide to the 3′ end of the antisense oligonucleotide form the targeting sequence;
at least one subunit is an abasic subunit; and
wherein the targeting sequence, except for the abasic subunit or subunits, is at least 80% complementary to the target region.

As noted above, “GSD-II” refers to glycogen storage disease type II (GSD-II or Pompe disease), a human autosomal recessive disease that is often characterized by underexpression of GAA protein in affected individuals. Included are subjects having infantile GSD-II and those having late-onset forms of the disease.

In certain embodiments, a subject has reduced expression and/or activity of GAA protein in one or more tissues (for example, relative to a healthy subject or an earlier point in time), including heart, skeletal muscle, liver, and nervous system tissues. In some embodiments, the subject has increased accumulation of glycogen in one or more tissues (for example, relative to a healthy subject or an earlier point in time), including heart, skeletal muscle, liver, and nervous system tissues. In specific embodiments, the subject has at least one IVS1-13T>G mutation (also referred to as c.336-13T>G), possibly in combination with other mutation(s) that leads to reduced expression of functional GAA protein. A summary of molecular genetic testing used in GSD-II is shown in Table 5 below.

TABLE 5

| | | | Mutation | |
|---|---|---|---|---|
| | | | Detection | |
| Gene | | | Frequency by | Test |
| Symbol | Test Method | Mutations Detected | Test Method | Availability |
| GAA | Sequence analysis | p. Arg854* | ~50%-60% | Clinical |
| | | p. Asp645Glu | ~40%-80% | |
| | | IVS1-13T > G | ~50%-85% | |
| | | Other sequence variants in | 83%-93% | |
| | | the gene | | |
| | Sequence analysis of | Sequence variants in the | 83%-93% | |
| | select exons | select exons | | |
| | Targeted mutation | Sequence variants in | 100% of for | |
| | analysis | targeted sites | variants among | |
| | | | the targeted | |
| | | | mutations | |
| | Deletion/duplication | Exonic and whole-gene | 5%-13% | |
| | analysis | deletions/duplications | | |

Certain embodiments relate to methods of increasing expression of exon 2-containing GAA mRNA or protein in a cell, tissue, and/or subject, as described herein. In some instances, exon-2 containing GAA mRNA or protein is increased by about or at least about 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 95%, or 100% relative to a control, for example, a control cell/subject, a control composition without the antisense oligomer, the absence of treatment, and/or an earlier time-point. Also included are methods of maintaining the expression of containing GAA mRNA or protein relative to the levels of a healthy control.

Some embodiments relate to methods of increasing expression of functional/active GAA protein in a cell, tissue, and/or subject, as described herein. In certain instances, the level of functional/active GAA protein is increased by about or at least about 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 95%, or 100% relative to a control, for example, a control cell/subject, a control composition without the antisense oligomer, the absence of treatment, and/or an earlier time-point. Also included are methods of maintaining the expression of functional/active GAA protein relative to the levels of a healthy control.

Particular embodiments relate to methods of reducing the accumulation of glycogen in one or more cells, tissues, and/or subjects, as described herein. In certain instances, the accumulation of glycogen is reduced by about or at least about 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 95%, or 100% relative to a control, for example, a control cell/subject, a control composition without the antisense oligomer, the absence of treatment, and/or an earlier time-point. Also included are methods of maintaining normal or otherwise healthy glycogen levels in a cell, tissue, and/or subject (e.g., asymptomatic levels or levels associated with reduced symptoms of GSD-II).

Also included are methods of reducing one or more symptoms of GSD-II in a subject in need thereof. Particular examples include symptoms of infantile GSD-II such as cardiomegaly, hypotonia, cardiomyopathy, left ventricular outflow obstruction, respiratory distress, motor delay/muscle weakness, and feeding difficulties/failure to thrive. Additional examples include symptoms of late-onset GSD-II such as muscle weakness (e.g., skeletal muscle weakness including progressive muscle weakness), impaired cough, recurrent chest infections, hypotonia, delayed motor milestones, difficulty swallowing or chewing, and reduced vital capacity or respiratory insufficiency.

The antisense oligomers of the disclosure can be administered to subjects to treat (prophylactically or therapeutically) GSD-II. In conjunction with such treatment, pharmacogenomics (i.e., the study of the relationship between an individual's genotype and that individual's response to a foreign compound or drug) may be considered. Differences in the metabolism of therapeutics can lead to severe toxicity or therapeutic failure by altering the relation between dose and blood concentration of the pharmacologically active drug.

Thus, a physician or clinician may consider applying knowledge obtained in relevant pharmacogenomics studies in determining whether to administer a therapeutic agent as well as tailoring the dosage and/or therapeutic regimen of treatment with a therapeutic agent.

Effective delivery of the antisense oligomer to the target nucleic acid is one aspect of treatment. Routes of antisense oligomer delivery include, but are not limited to, various systemic routes, including oral and parenteral routes, e.g., intravenous, subcutaneous, intraperitoneal, and intramuscular, as well as inhalation, transdermal, and topical delivery. The appropriate route may be determined by one of skill in the art, as appropriate to the condition of the subject under treatment. Vascular or extravascular circulation, the blood or lymph system, and the cerebrospinal fluid are some non-limiting sites where the RNA may be introduced. Direct CNS delivery may be employed, for instance, intracerebral ventricular or intrathecal administration may be used as routes of administration.

In particular embodiments, the antisense oligomer(s) are administered to the subject by intramuscular injection (IM), i.e., they are administered or delivered intramuscularly. Non-limiting examples of intramuscular injection sites include the deltoid muscle of the arm, the vastus lateralis muscle of the leg, and the ventrogluteal muscles of the hips, and dorsogluteal muscles of the buttocks. In specific embodiments, a PMO, PMO-X, or PPMO is administered by IM.

In certain embodiments, the subject in need thereof as glycogen accumulation in central nervous system tissues. Examples include instances where central nervous system pathology contributes to respiratory deficits in GSD-II (see, e.g., DeRuisseau et al., PNAS USA. 106:9419-24, 2009). Accordingly, the antisense oligomers described herein can be delivered to the nervous system of a subject by any art-recognized method, e.g., where the subject has GSD-II with involvement of the CNS. For example, peripheral blood injection of the antisense oligomers of the disclosure can be used to deliver said reagents to peripheral neurons via diffusive and/or active means. Alternatively, the antisense oligomers can be modified to promote crossing of the blood-brain-barrier (BBB) to achieve delivery of said reagents to neuronal cells of the central nervous system (CNS). Specific recent advancements in antisense oligomer technology and delivery strategies have broadened the scope of antisense oligomer usage for neuronal disorders (see, e.g., Forte, A., et al. 2005. Curr. Drug Targets 6:21-29; Jaeger, L. B., and W. A. Banks. 2005. Methods Mol. Med. 106:237-251; Vinogradov, S. V., et al. 2004. Bioconjug. Chem. 5:50-60; the foregoing are incorporated herein in their entirety by reference). For example, the antisense oligomers of the disclosure can be generated as peptide nucleic acid (PNA) compounds. PNA reagents have each been identified to cross the BBB (Jaeger, L. B., and W. A. Banks. 2005. Methods Mol. Med. 106:237-251). Treatment of a subject with, e.g., a vasoactive agent, has also been described to promote transport across the BBB (id). Tethering of the antisense oligomers of the disclosure to agents that are actively transported across the BBB may also be used as a delivery mechanism. Administration of antisense agents together with contrast agents such as iohexol (e.g., separately, concurrently, in the same formulation) can also facilitate delivery across the BBB, as described in PCT Publication No. WO/2013/086207, incorporated by reference in its entirety.

In certain embodiments, the antisense oligomers of the disclosure can be delivered by transdermal methods (e.g., via incorporation of the antisense oligomers into, e.g., emulsions, with such antisense oligomers optionally packaged into liposomes). Such transdermal and emulsion/liposome-mediated methods of delivery are described for delivery of antisense oligomers in the art, e.g., in U.S. Pat. No. 6,965,025, the contents of which are incorporated in their entirety by reference herein.

The antisense oligomers described herein may also be delivered via an implantable device. Design of such a device is an art-recognized process, with, e.g., synthetic implant design described in, e.g., U.S. Pat. No. 6,969,400, the contents of which are incorporated in their entirety by reference herein.

Antisense oligomers can be introduced into cells using art-recognized techniques (e.g., transfection, electroporation, fusion, liposomes, colloidal polymeric particles, and viral and non-viral vectors as well as other means known in the art). The method of delivery selected will depend at least on the oligomer chemistry, the cells to be treated and the location of the cells and will be apparent to the skilled artisan. For instance, localization can be achieved by liposomes with specific markers on the surface to direct the liposome, direct injection into tissue containing target cells, specific receptor-mediated uptake, or the like.

As known in the art, antisense oligomers may be delivered using, e.g., methods involving liposome-mediated uptake, exosome-mediated uptake, lipid conjugates, polylysine-mediated uptake, nanoparticle-mediated uptake, and receptor-mediated endocytosis, as well as additional non-endocytic modes of delivery, such as microinjection, permeabilization (e.g., streptolysin-O permeabilization, anionic peptide permeabilization), electroporation, and various non-invasive non-endocytic methods of delivery that are known in the art (refer to Dokka and Rojanasakul, Advanced Drug Delivery Reviews 44, 35-49, incorporated by reference in its entirety).

The antisense oligomers may be administered in any convenient vehicle or carrier which is physiologically and/or pharmaceutically acceptable. Such a composition may include any of a variety of standard pharmaceutically acceptable carriers employed by those of ordinary skill in the art. Examples include, but are not limited to, saline, phosphate-buffered saline (PBS), water, aqueous ethanol, emulsions, such as oil/water emulsions or triglyceride emulsions, tablets, and capsules. The choice of a suitable physiologically acceptable carrier will vary dependent upon the chosen mode of administration. “Pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

The compounds (e.g., antisense oligomers) of the present disclosure may generally be utilized as the free acid or free base. Alternatively, the compounds of this disclosure may be used in the form of acid or base addition salts. Acid addition salts of the free amino compounds of the present disclosure may be prepared by methods well known in the art and may be formed from organic and inorganic acids. Suitable organic acids include maleic, fumaric, benzoic, ascorbic, succinic, methanesulfonic, acetic, trifluoroacetic, oxalic, propionic, tartaric, salicylic, citric, gluconic, lactic, mandelic, cinnamic, aspartic, stearic, palmitic, glycolic, glutamic, and benzenesulfonic acids.

Suitable inorganic acids include hydrochloric, hydrobromic, sulfuric, phosphoric, and nitric acids. Base addition salts included those salts that form with the carboxylate anion and include salts formed with organic and inorganic cations such as those chosen from the alkali and alkaline earth metals (for example, lithium, sodium, potassium, magnesium, barium and calcium), as well as the ammonium ion and substituted derivatives thereof (for example, dibenzylammonium, benzylammonium, 2-hydroxyethylammonium, and the like). Thus, the term “pharmaceutically acceptable salt” is intended to encompass any and all acceptable salt forms.

In addition, prodrugs are also included within the context of this disclosure. Prodrugs are any covalently bonded carriers that release a compound in vivo when such prodrug is administered to a patient. Prodrugs are generally prepared by modifying functional groups in a way such that the modification is cleaved, either by routine manipulation or in vivo, yielding the parent compound. Prodrugs include, for example, compounds of this disclosure wherein hydroxy, amine, or sulfhydryl groups are bonded to any group that, when administered to a patient, cleaves to form the hydroxy, amine, or sulfhydryl groups. Thus, representative examples of prodrugs include (but are not limited to) acetate, formate, and benzoate derivatives of alcohol and amine functional groups of the antisense oligomers of the disclosure. Further, in the case of a carboxylic acid (—COOH), esters may be employed, such as methyl esters, ethyl esters, and the like.

In some instances, liposomes may be employed to facilitate uptake of the antisense oligomer into cells (see, e.g., Williams, S. A., Leukemia 10(12):1980-1989, 1996; Lappalainen et al., Antiviral Res. 23:119, 1994; Uhlmann et al., antisense oligomers: a new therapeutic principle, Chemical Reviews, Volume 90, No. 4, 25 pages 544-584, 1990; Gregoriadis, G., Chapter 14, Liposomes, Drug Carriers in Biology and Medicine, pp. 287-341, Academic Press, 1979). Hydrogels may also be used as vehicles for antisense oligomer administration, for example, as described in WO 93/01286. Alternatively, the oligomers may be administered in microspheres or microparticles. (See, e.g., Wu, G. Y. and Wu, C. H., J. Biol. Chem. 262:4429-4432, 30 1987). Alternatively, the use of gas-filled microbubbles complexed with the antisense oligomers can enhance delivery to target tissues, as described in U.S. Pat. No. 6,245,747. Sustained-release compositions may also be used. These may include semipermeable polymeric matrices in the form of shaped articles such as films or microcapsules.

In one embodiment, the antisense oligomer is administered to a mammalian subject, e.g., human or domestic animal, exhibiting the symptoms of a lysosomal storage disorder, in a suitable pharmaceutical carrier. In one aspect of the method, the subject is a human subject, e.g., a patient diagnosed as having GSD-II (Pompe disease). In one preferred embodiment, the antisense oligomer is contained in a pharmaceutically acceptable carrier and is delivered orally. In another preferred embodiment, the oligomer is contained in a pharmaceutically acceptable carrier and is delivered intravenously (i.v.).

In one embodiment, the antisense compound is administered in an amount and manner effective to result in a peak blood concentration of at least 200-400 nM antisense oligomer. Typically, one or more doses of antisense oligomer are administered, generally at regular intervals, for a period of about one to two weeks. Preferred doses for oral administration are from about 1-1000 mg oligomer per 70 kg. In some cases, doses of greater than 1000 mg oligomer/patient may be necessary. For i.v. administration, preferred doses are from about 0.5 mg to 1000 mg oligomer per 70 kg. The antisense oligomer may be administered at regular intervals for a short time period, e.g., daily for two weeks or less. However, in some cases the oligomer is administered intermittently over a longer period of time. Administration may be followed by, or concurrent with, administration of an antibiotic or other therapeutic treatment. The treatment regimen may be adjusted (dose, frequency, route, etc.) as indicated, based on the results of immunoassays, other biochemical tests, and physiological examination of the subject under treatment.

In certain embodiments, the method is an in vitro method. In certain other embodiments, the method is an in vivo method.

In certain embodiments, the host cell is a mammalian cell. In certain embodiments, the host cell is a non-human primate cell. In certain embodiments, the host cell is a human cell.

In certain embodiments, the host cell is a naturally occurring cell. In certain other embodiments, the host cell is an engineered cell.

In certain embodiments, the antisense oligomer is administered to a mammalian subject, e.g., a human or a laboratory or domestic animal, in a suitable pharmaceutical carrier.

In certain embodiments, the antisense oligomer is administered to a mammalian subject, e.g., a human or laboratory or domestic animal, together with an additional agent. The antisense oligomer and the additional agent can be administered simultaneously or sequentially, via the same or different routes and/or sites of administration. In certain embodiments, the antisense oligomer and the additional agent can be co-formulated and administered together. In certain embodiments, the antisense oligomer and the additional agent can be provided together in a kit.

In one embodiment, the antisense oligomer, contained in a pharmaceutically acceptable carrier, is delivered orally.

In one embodiment, the antisense oligomer, contained in a pharmaceutically acceptable carrier, is delivered intravenously (i.v.).

Additional routes of administration, e.g., subcutaneous, intraperitoneal, and pulmonary, are also contemplated by the instant disclosure.

In another application of the method, the subject is a livestock animal, e.g., a pig, cow, or goat, etc., and the treatment is either prophylactic or therapeutic. Also contemplated is, in a method of feeding livestock with a food substance, an improvement in which the food substance is supplemented with an effective amount of an antisense oligomer composition as described above.

In an embodiment, the antisense oligomer is administered in an amount and manner effective to result in a peak blood concentration of at least 200 nM antisense oligomer. In one embodiment, the antisense oligomer is administered in an amount and manner effective to result in a peak plasma concentration of at least 200 nM antisense oligomer. In one embodiment, the antisense oligomer is administered in an amount and manner effective to result in a peak serum concentration of at least 200 nM antisense oligomer.

In an embodiment, the antisense oligomer is administered in an amount and manner effective to result in a peak blood concentration of at least 400 nM antisense oligomer. In one embodiment, the antisense oligomer is administered in an amount and manner effective to result in a peak plasma concentration of at least 400 nM antisense oligomer. In one embodiment, the antisense oligomer is administered in an amount and manner effective to result in a peak serum concentration of at least 400 nM antisense oligomer.

Typically, one or more doses of the antisense oligomer are administered, generally at regular intervals, for a period of about one to two weeks. Preferred doses for oral administration are from about 0.01-15 mg antisense oligomer per kg body weight. In some cases, doses of greater than 15 mg antisense oligomer/kg may be necessary. For i.v. administration, preferred doses are from about 0.005 mg to 15 mg antisense oligomer per kg body weight. The antisense oligomer may be administered at regular intervals for a short time period, e.g., daily for two weeks or less. However, in some cases, the antisense oligomer is administered intermittently over a longer period of time. Administration may be followed by or accompanied by, administration of an antibiotic or other therapeutic treatment. The treatment regimen may be adjusted (dose, frequency, route, etc.) as indicated, based on the results of immunoassays, other biochemical tests, and physiological examination of the subject under treatment.

An effective in vivo treatment regimen using the antisense oligomer may vary according to the duration, dose, frequency, and route of administration, as well as the condition of the subject under treatment (i.e., prophylactic administration versus administration in response to localized or systemic infection). Accordingly, such in vivo therapy will often require monitoring by tests under treatment, and corresponding adjustments in the dose or treatment regimen, in order to achieve an optimal therapeutic outcome.

In some embodiments, the antisense oligomer is actively taken up by mammalian cells. In further embodiments, the antisense oligomer can be conjugated to a transport moiety (e.g., transport peptide) as described herein to facilitate such uptake.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### EXAMPLES

Examples have been set forth below for the purpose of illustration and to describe certain specific embodiments of the disclosure. However, the scope of the claims is not to be in any way limited by the examples set forth herein. Various changes and modifications to the disclosed embodiments will be apparent to those skilled in the art and such changes and modifications including, without limitation, those relating to the chemical structures, substituents, derivatives, formulations, or methods of the disclosure may be made without departing from the spirit of the disclosure and the scope of the appended claims. Definitions of the variables in the structures in the schemes herein are commensurate with those of corresponding positions in the formulae presented herein.

### Example 1—Design of Antisense Targeting Sequences

Antisense oligomer targeting sequences were designed for therapeutic splice-switching applications related to the IVS1-13T>G mutation in the human GAA gene. Here, it is expected that splice-switching oligomers will suppress intronic and exonic splice silencer elements (ISS and ESS elements, respectively) and thereby promote exon 2 retention in the mature GAA mRNA. Restoration of normal or near-normal GAA expression would then allow the functional enzyme to be synthesized, thereby providing a clinical benefit to GSD-II patients.

Exemplary oligomers comprising a targeting sequence as set forth in Tables 6A-6C were prepared as PPMOs (oligomers conjugated to a CPP, such as an arginine-rich CPP). As described below, these antisense oligomers were introduced into GSD-II patient-derived fibroblasts and patient iPSC-derived myotubes using a gymnotic uptake protocol as also described in Example 2 below.

### Example 2—Materials and Methods

GSD-II cells. Patient-derived fibroblasts from individuals with GSD-II (Coriell cell lines GM00443 and GM11661) were cultured according to standard protocols in Eagle's DMEM with 10%-15% FBS. Cells were passaged at least twice before the experiments and are approximately 80% confluent at transfection. GM00443 and GM11661 patient-derived fibroblasts were reprogrammed to iPSC lines and subsequently differentiated to myoblasts and expanded and banked. Patient iPSC-derived myoblasts were cultured in myoblast expansion media and passaged twice before use. Myoblasts were differentiated to myotubes for two days before treatment.

GM00443 fibroblasts are from a 30-year-old male. Adult form; onset in the third decade; normal size and amount of mRNA for GAA, GAA protein detected by antibody, but only 9 to 26% of normal acid-alpha-1,4 glucosidase activity; passage 3 at CCR; donor subject is heterozygous with one allele carrying a T>G transversion at position −13 of the acceptor site of intron 1 of the GAA gene, resulting in alternatively spliced transcripts with deletion of the first coding exon [exon 2 (IVS1-13T>G)].

GM11661 fibroblasts are from a 38-year-old male. Abnormal liver function tests; occasional charley-horse in legs during physical activity; morning headaches; intolerance to greasy foods; abdominal cyst; deficient fibroblast and WBC acid-alpha-1,4 glucosidase activity; donor subject is a compound heterozygote: allele one carries a T>G transversion at position −13 of the acceptor site of intron 1 of the GAA gene (IVS1-13T>G); the resulting alternatively spliced transcript has an in-frame deletion of exon 2 which contains the initiation codon; allele two carries an in-frame deletion of exon 18.

Treatment protocol. Patient-derived fibroblasts were passaged twice before use. Cells were treated at around 80% confluency by changing media containing various concentrations of PPMO. Patient iPSC-derived myoblasts were passaged/expanded at least twice before use. Myoblasts were cultured for one day and differentiated to myotubes for two days before treatment with differentiation media containing various concentrations of PPMO.

GAA qPCR. For quantitative PCR experiments, a multiplex TaqMan qPCR assay was used that simultaneously amplifies GAA mRNA at exon 1-2 and exon 3-4 junctions in addition to a reference gene. 100-500 ng of total RNA from treated patient iPSC-derive myotubes was reverse transcribed using the SuperScript VILO cDNA synthesis Kit (Thermo Fisher). cDNA was diluted 3-10 fold before amplification using TaqMan Multiplex Master Mix (Thermo Fisher) using a Quantstuio 7 Pro thermocycler (Thermo Fisher). Each qPCR reaction contained a FAM probe to detect GAA exon 1-2 junction, a VIC probe to detect GAA exon 3-4 junction, and a JUN probe to detect a reference gene. Relative standard curves for each assay and probe set in multiplex were generated and used to calculate the starting quantity of each species in treated samples normalized to the reference gene.

GAA Enzyme Assay & Protein Simple Wes. Patient-derived fibroblasts were cultured to about 80% confluency and then treated with PPMO compounds via gymnotic uptake. Treatment was continued for 6 days at which time GAA activity was measured using the Abcam GAA Activity Assay Kit (ab252887).

A Western blot on GAA protein was performed using the ProteinSimple® Jes™ System. GAA was detected using recombinant anti-GAA antibody (EPR4716(2))(Abcam ab137068) and the ProteinSimple® anti-rabbit detection module (DM-001) and 12-230 kDa separation module (SM-W004). GAA protein concentrations were normalized to total protein using the ProteinSimple® Protein normalization Kit (AM-PN01)

### Example 3—Preparation of Antisense PPMOs (R¹ is —N(CH₃)₂)

Antisense PPMOs were designed to target the human GAA pre-mRNA (e.g., intron 1 of the human GAA pre-mRNA) were synthesized as described herein and used to treat GSD-II patient-derived fibroblasts and GSD-II patient iPSC-derived myotubes.

TABLE 6A
Nucleofected PPMO Compounds (Abasic subunits)

| | | TS | |
|---|---|---|---|
| | Targeting Sequence (TS)* | SEQ | 5′ terminus |
| Name | (5′-3′) | ID NO | 3′ terminus |
| GAA-IVS1 (-189, 167) | CCA GAA GGA AGG GCG AGA AAA GC | 33 | 5′ TEG |
| | | | 3′ GR6 |
| GAA-IVS1 (-189, | CCA GAA GGA AGG CGA GAA AAG C | 34 | 5′ TEG |
| 167)-G | | | 3′ GR6 |
| GAA-IVS1 (-189, | CCA GAA GGA AGC GAG AAA AGC | 35 | 5′ TEG |
| 167)-2G | | | 3′ GR6 |
| H53A (-100-76) | CGT TAT CTC ACA TTT ATG TTG CTT A | NTC | 5′ TEG |
| Negative Control | | | 3′ GR6 |
| (NTC) | | | |
| GAA-IVS1 (-189, 167) | CCA GAA GGA AGG BCG AGA AAA GC | 5 | 5′ TEG |
| (-179 Abasic) | | | 3′ GR6 |
| GAA-IVS1 (-189, 167) | CCA GAA GGA AGB GCG AGA AAA GC | 6 | 5′ TEG |
| (-178 Abasic) | | | 3′ GR6 |
| GAA-IVS1 (-189, 167) | CCA GAA GGA ABG GCG AGA AAA GC | 7 | 5′ TEG |
| (-177 Abasic) | | | 3′ GR6 |
| GAA-IVS1 (-189, 167) | CCA GAA GGA AGB BCG AGA AAA GC | 8 | 5′ TEG |
| (-178,-179 Abasic) | | | 3′ GR6 |
| GAA-IVS1 (-189, 167) | CCA GAA GGA ABB GCG AGA AAA GC | 9 | 5′ TEG |
| (-177,-178 Abasic) | | | 3′ GR6 |
| GAA-IVS1 (-189, 167) | CCA GAA GGA ABG BCG AGA AAA GC | 10 | 5′ TEG |
| (-177,-179 Abasic) | | | 3′ GR6 |

GR6: Gly(Arg)₆ B: a purine and pyrimidine-free abasic subunit. The abasic subunits incorporated herein retain the phosphorodiamidate backbone of the oligomer but do not contain purine or pyrimidine bases.

TABLE 6B
Microwalk Data

| | | TS | |
|---|---|---|---|
| | Targeting Sequence (TS)* | SEQ | 5′ terminus |
| Name | (5′-3′) | ID NO | 3′ terminus |
| GAA-IVS1 (-80, -56) | GCT CTC AAA GCA GCT CTG AGA CAT C | 37 | 5′ TEG |
| | | | 3′ GR6 |
| GAA-IVS1 (-76, -52) | CGG GGC TCT CAA AGC AGC TCT GAG A | 38 | 5′ TEG |
| | | | 3′ GR6 |
| GAA-IVS1 (-74, -55) | GGC TCT CAA AGC AGC TCT GA | 39 | 5′ TEG |
| | | | 3′ R6 |
| GAA-IVS1 (-72, -48) | CTC ACG GGG CTC TCA AAG CAG CTC T | 40 | 5′ TEG |
| | | | 3′ GR6 |
| GAA-IVS1 (-71, -47) | ACT CAC GGG GCT CTC AAA GCA GCT C | 41 | 5′ TEG |
| | | | 3′ GR6 |
| GAA-IVS1 (-70, -46) | CAC TCA CGG GGC TCT CAA AGC AGC T | 42 | 5′ TEG |
| | | | 3′ GR6 |
| GAA-IVS1 (-69, -45) | GCA CTC ACG GGG CTC TCA AAG CAG C | 43 | 5′ TEG |
| | | | 3′ GR6 |
| GAA-IVS1 (-66, -42) | GGC GGC ACT CAC GGG GCT CTC AAA G | 44 | 5′ TEG |
| | | | 3′ GR6 |
| GAA-IVS1 (-65, -41) | GGC GGC ACT CAC GGC TCT CAA AG | 45 | 5′ TEG |
| -2G | | | 3′ GR6 |
| GAA-IVS1 (-49, -24) | GCA GGG AGG CGG GAG GGG CGG CAC T | 46 | 5′ TEG |
| | | | 3′ GR6 |

GR6: Gly(Arg)₆

TABLE 6C
Nucleofected PPMO Compounds (Abasic subunits)

| | | TS | |
|---|---|---|---|
| | Targeting Sequence (TS)* | SEQ | 5′ Attachment |
| Name | (5′-3′) | ID NO | 3′ Attachment |
| GAA h5′Ex1 | TCC TAC CTG CTG CCT CAT | 47 | 5′ TEG |
| (173 190) | | | 3′ GR6 |
| GAA-IVS1 (-69-45) | GCA CTC ACB GGG CTC TCA AAG CAG C | 17 | 5′ TEG |
| (G53B) | | | 3′ GR6 |
| GAA-IVS1 (-69-45) | GCA CTC ACG BGG CTC TCA AAG CAG C | 18 | 5′ TEG |
| (G54B) | | | 3′ GR6 |
| GAA-IVS1 (-69-45) | GCA CTC ACG GBG CTC TCA AAG CAG C | 19 | 5′ TEG |
| (G55B) | | | 3′ GR6 |
| GAA-IVS1 (-69-45) | GCA CTC ACG GGB CTC TCA AAG CAG C | 20 | 5′ TEG |
| (G56B) | | | 3′ GR6 |
| GAA-IVS1 (-69-45) | GCA CTC ACB BGG CTC TCA AAG CAG C | 21 | 5′ TEG |
| (G53B G54B) | | | 3′ GR6 |
| GAA-IVS1 (-69-45) | GCA CTC ACG BBG CTC TCA AAG CAG C | 22 | 5′ TEG |
| (G54B G55B) | | | 3′ GR6 |
| GAA-IVS1 (-69-45) | GCA CTC ACG GBB CTC TCA AAG CAG C | 23 | 5′ TEG |
| (G55B G56B) | | | 3′ GR6 |
| GAA-IVS1 (-65-41) | GGC GGC ACT CAC GBB GCT CTC AAA G | 24 | 5′ TEG |
| (G54B G55B) | | | 3′ GR6 |

GR6: Gly(Arg)₆ B: a purine and pyrimidine-free abasic subunit. The abasic subunits incorporated herein retain the phosphorodiamidate backbone of the oligomer but do not contain purine or pyrimidine bases.

TABLE 6D
Microwalk Data

| | | TS | |
|---|---|---|---|
| | Targeting Sequence (TS)* | SEQ | 5′ terminus |
| Name | (5′-3′) | ID NO | 3′ terminus |
| GAA-IVS1 (-186-166) | GCC AGA AGG AAG GGC GAG AAA | 48 | 5′ TEG |
| | | | 3′ GR6 |
| GAA-IVS1 (-188-168) | CAG AAG GAA GGG CGA GAA AAG | 49 | 5′ TEG |
| | | | 3′ GR6 |
| GAA-IVS1 (-189-169) | AGA AGG AAG GGC GAG AAA AGC | 50 | 5′ TEG |
| | | | 3′ R6 |
| GAA-IVS1 (-190-170) | GAA GGA AGG GCG AGA AAA GCT | 51 | 5′ TEG |
| | | | 3′ GR6 |
| GAA-IVS1 (-191-171) | AAG GAA GGG CGA GAA AAG CTC | 52 | 5′ TEG |
| | | | 3′ GR6 |
| GAA-IVS1 (-192-172) | AGG AAG GGC GAG AAA AGC TCC | 53 | 5′ TEG |
| | | | 3′ GR6 |
| GAA-IVS1 (-196-176) | AGG GCG AGA AAA GCT CCA GCA | 54 | 5′ TEG |
| | | | 3′ GR6 |
| GAA-IVS1 (-194-174) | GAA GGG CGA GAA AAG CTC CAG | 55 | 5′ TEG |
| | | | 3′ GR6 |
| GAA-IVS1 (-198-178) | GGC GAG AAA AGC TCC AGC AGG | 56 | 5′ TEG |
| | | | 3′ GR6 |

GR6: Gly(Arg)₆

### Example 4—GAA Activity and Protein in GSD-II Patient-Derived Fibroblasts

The above-described antisense PPMOs were delivered to GM00443 or GM11661 fibroblasts and After patient iPSC-derived myotubes by gymnotic uptake. Four to six days of incubation at 37° C. with 5% CO₂, cells were lysed and GAA activity in the lysates or GAA protein expression was measured by immunoassay as described above. In general, protein expression of GAA enzyme in cells treated with antisense oligonucleotides of the disclosure was higher than the GAA expression level in untreated cells. These results indicate that oligonucleotides of the disclosure increase expression and/or activity of the GAA enzyme in cells from patients with late-onset Pompe disease. The targeting sequences of the variant oligonucleotides are complementary to a target region within intron 1 (SEQ ID NO: 1) of a pre-mRNA of the human alpha-glucosidase (GAA) gene, wherein the target region comprise purine and pyrimidine-free abasic subunits. Surprisingly, the use of these abasic subunits facilitated the synthesis of low-yielding oligonucleotides while retaining the potency of the parental sequence.

### Example 5—Small-scale Synthesis of Activated Morpholino Abasic Subunit

General Preparation: Compound 1 (1.10 eq.) was suspended in dichloromethane (7.00 mL/g). To this suspension were added tetramethylguanidine (0.50 eq.) and diisopropylethylamine (1.80 eq.), and the mixture was warmed to 30° C. and held for 60 minutes to dissolve the material before cooling to room temperature. Separately, trityl chloride (1.00 eq.) was dissolved in dichloromethane (2.42 mL/g), and this solution was added slowly to the first solution, keeping the temperature under 30° C. On reaction completion (1-2 hrs.), the reaction mixture was washed with a citrate buffer (pH 4) and with water. The organic phase was separated and assayed for Compound 2 content (yield: 93%).

To the solution of Compound 2 (1.00 eq.) were added 2,6-lutidine (1.15 eq.) and N-methylimidazole (0.38 eq.), and the solution was concentrated by atmospheric distillation to a volume of 6.00 mL/g. Dichloromethane (5.00 mL/g) was added to the solution, and it was again concentrated by atmospheric distillation to the same volume. This process was repeated until water content was undetectable by Karl-Fischer titration, and the solution was chilled to 0-5° C. Dimethylaminophosphoryl dichloride (1.05 eq.) was added in a thin stream, and the reaction mixture was allowed to warm to room temperature overnight.

After confirmation of reaction completion, the mixture was passed through a column of molecular sieves, and the resulting solution was purified by silica gel chromatography using step gradients of ethyl acetate in heptanes. Product-containing fractions were pooled, and the pool was evaporated to dryness to produce the final product solids (yield: 73% for the final step and 68% for the two-step process).

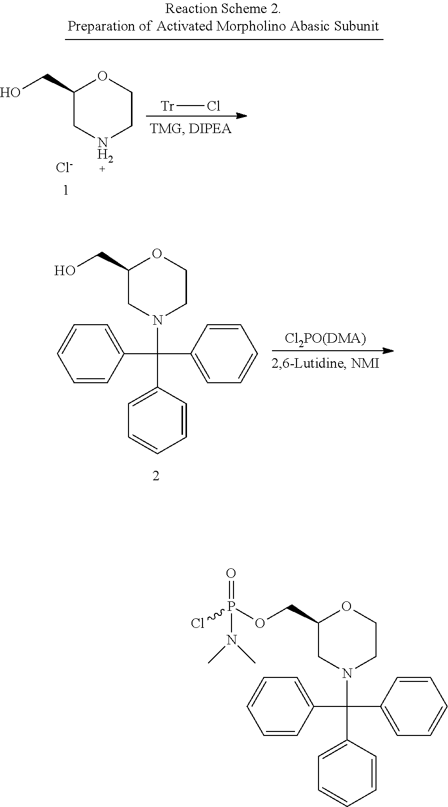

Since the activated morpholino subunits are unstable in aqueous HPLC mobile phases, a quench derivatization with 1-(4-nitrophenyl)piperazine (NPP) was employed to convert the subunit into stable diamidate derivatives for analysis with HPLC. NPP absorbs strongly at 391 nm, so, in addition to providing stability, analysis at 391 nm can be used to view impurities likely to react with the growing chain of the oligomer.

Since the product for analysis is the NPP-derivatized activated morpholino basic subunit, a standard of this material was synthesized and characterized with HPLC, mass spec, and NMR. This allows identification of the activated subunit by comparison against the HPLC chromatogram of the synthesized standard, which has mass spec and NMR confirming structure. The product peak in the HPLC analyses is slightly split due to the partial resolution of the two diastereomers.

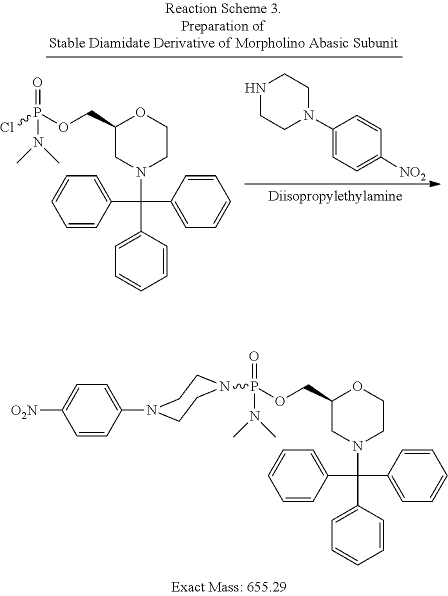 [SMILES: CN(C)P(=O)(Cl)OC[C@@H]1CN(C(c2ccccc2)(c2ccccc2)c2ccccc2)CCO1 ; O=[N+]([O-])c1ccc(N2CCNCC2)cc1 ; CN(C)P(=O)(OC[C@@H]1CN(C(c2ccccc2)(c2ccccc2)c2ccccc2)CCO1)N1CCN(c2ccc([N+](=O)[O-])cc2)CC1]

### Example 6—GAA Activity in GSD-II Patient-Derived Fibroblasts

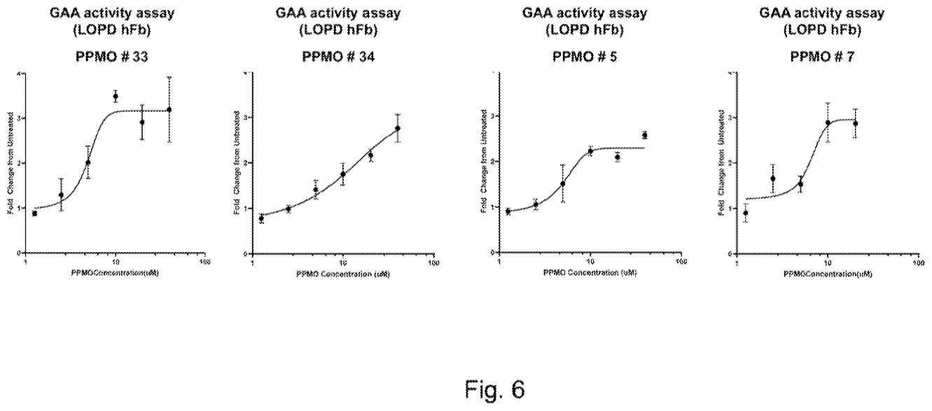

Fibroblast cell cultures. Human fibroblast cell lines were maintained in modified eagle medium (MEM, Thermo Fisher) containing 15% Fetal Bovine Serum (FBS) and 2 mM L-glutamine at 37° C. incubator with 5% CO₂. Fibroblast cell lines currently used were obtained from Coriell Institute and include the following lines: GM08402 (healthy control), GM08400 (healthy control), GM00443 (Pompe late-onset), GM11661 (Pompe late-onset), GM20089 (Pompe infantile onset), and GM20123 (healthy Pompe carrier). One day before treatment cells were plated in 24-well cell culture plates at 30,000 cells/well and incubated overnight. Cells were then washed in PBS and a treatment of PPMO supplemented with media was added to wells. Cells were allowed to incubate with treatment and no media changes for 6 days. For GAA Activity Assay lysis, cells were washed once with PBS and then lysed in ice-cold GAA Activity Assay Buffer (Abcam) FIG. 6 shows a dose dependent increase of GAA expression in patient fibroblasts after gymnotic treatment with select PPMO compounds.

### Example 7—GAA Activity and Protein in GSD-II Patient-Derived Myotubes

Patient iPSC-derived myotubes. Patient fibroblasts were reprogrammed to iPSCs using a feeder-free and footprint-free method. Pluripotency was validated by immunostaining with markers Oct3/4, NANOG, TRA-1-60. iPSCs retained normal karyotype and alkaline phosphatase activity. iPSC lines were differentiated to myoblasts, frozen, and revived. Myogenic lineage was confirmed by immunofluorescence of the myoblast markers Desmin and MyoD and expression of key markers as measured by qPCR. Terminal differentiation of the myoblasts was performed over 3-6 days of culturing and confirmed by expression of the myogenic markers MHC and MyoG measured by immunofluorescence.

Non-integrative reprogramming of fibroblasts into iPSC. The Fibroblasts were maintained in DMEM 10% FCS. After overnight incubation, culture medium was replaced with a fresh one, and cells were transfected with 2 μg of episomal plasmids from Epi5™ iPSC Reprogramming Kit (Themofisher) by using FuGENE6 transfection reagent (Promega). The next day, culture medium was replaced with mTeSR-plus medium (StemCell Technologies). During the reprogramming process, transfected cells were cultured in mTeSR plus, and the medium was changed every other day up to 2 weeks post-transfection. Colonies were transferred onto new culture dishes covered with Geltrex matrix by using a pipette tip. An hour before the procedure, 10 μM Y-27632 was added to the culture medium. The iPSCs were further propagated and maintained in mTeSR plus medium as described in Alonso-Barroso et al., Stem Cell Res. 23, 173-177; 2017

SKM differentiation. Myogenic progenitors were differentiated from hiPSCs according to the protocols described previously [Chal, J et. Al. Nat. Biotech. 2015, 33, 962-969]. Briefly, myogenic progenitors were generated through a multi-step small molecule differentiation protocol. Myogenic progenitors were expanded, passaged, and cryopreserved in 60 μg/mL Collagen I coated 6-well plates. For myoblast differentiation, frozen myogenic progenitors were thawed in myoblast expansion medium (iXCells, Cat. #MD-0102A). Growth medium was refreshed every 2 days for 8 days then cryopreserved. For myotube differentiation, the myoblasts were recovered and seeded at a density of 32,000/cm2 and cultured using myoblast expansion medium to reach 100 percent confluency. For skeletal muscle cell differentiation, confluent myoblast cultures were switched over to myoblast differentiation medium (iXCells, Cat. #MD-0102B) with media changes every 2 days. Elongated myotubes were evident after 72 hours in myoblast differentiation medium.

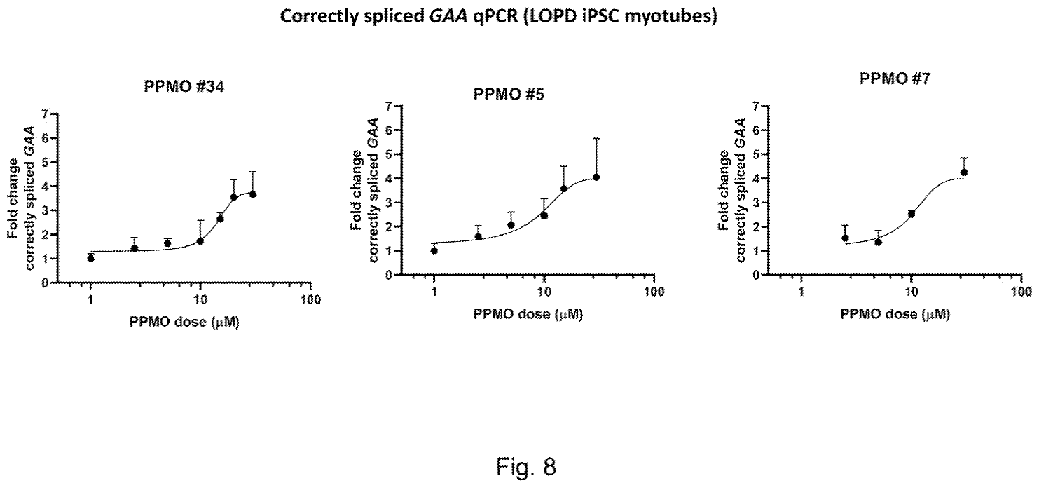

PPMO increase GAA expression in LOPD patient iPSC-derived myotubes. Patient iPSC-derived myoblasts were seeded in a 96-well or 24-well collagen coated plate (Corning) and expanded in iPSC-derived myoblast expansion medium (iXCells Biotechnologies) for 48 hours. Media was changed to myotube differentiation media (iXCells Biotechnologies) and differentiation was continued for 48 hours. Media was then changed to fresh differentiation media containing the indicated concentrations of PPMO. RNA was extracted from cell cultures after 72 hours of gymnotic treatment using the Quick-RNA 96 Kit (Zymo) following the manufacture's protocol. 100-300 ng of RNA was reverse transcribed using the superscript VILO kit (Thermo Fisher) according to the manufacturers protocol. A multiplex qPCR assay measuring GAA expression at the exon 1-2 locus (Hs.PT.58.24962380, Integrated DNA Technologies, 900 nM primers, 250 nM probe) on the FAM channel, GAA at the exon 3-4 locus (Hs01089834_m1, Thermo Fisher, 1.8 μM primers, 500 nM probe) on the VIC channel, and HPRT (Hs99999909_m1_qsy, Thermo Fisher, 900 nM primers, 250 nM probe) on the JUN channel was used with Multiplex Master Mix (Thermo Fisher) on a Quantstudio 7 Pro PCR thermocycler (Thermo Fisher). qPCR cycling conditions consisted of an initial denaturation step for 20 sec at 95° C., followed by 40 cycles of 95° C. for 3 s, 58° C. for 20 s with a 1.92° C. per second ramp rate. FIG. 7 and FIG. 8 show dose dependent increases of GAA expression in patient iPSC-derived myotubes after gymnotic treatment with selected PPMOs.

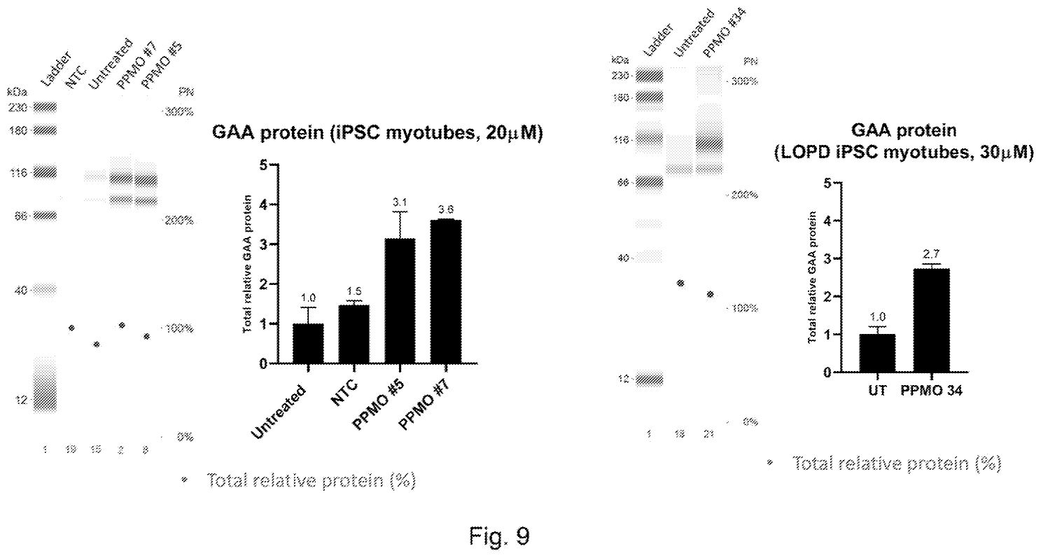

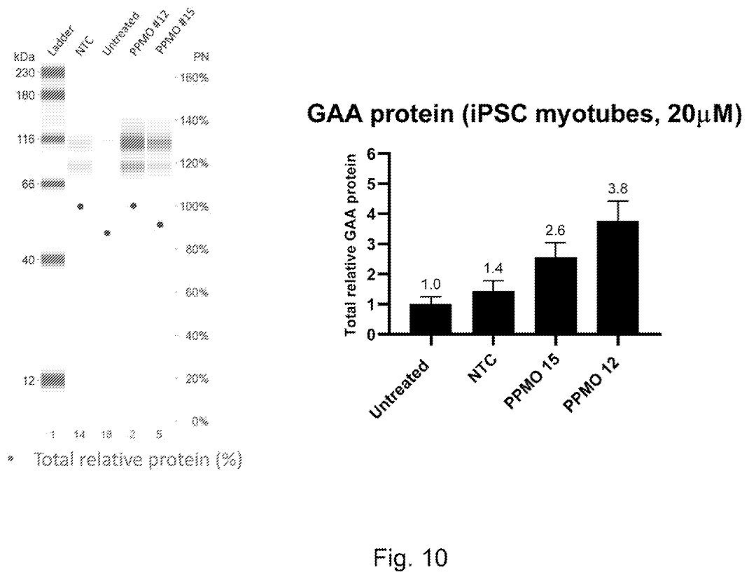

PPMO increase GAA protein in LOPD patient iPSC-derived myotubes. Patient iPSC-derived myoblasts were seeded in a 24-well collagen coated plate (Corning) and expanded in iPSC-derived myoblast expansion medium (iXCells Biotechnologies) for 24 hours. Media was changed to myotube differentiation media (iXCells Biotechnologies) and differentiation was continued for 24 hours. Media was then changed to fresh differentiation media containing the indicated concentrations of PPMO. Cell lysates were prepared after 96 hours of gymnotic treatment using RIPA lysis buffer (Thermo Fisher). Protein concentration was measured using Pierce BCA Assay Kit (Thermo Fisher). Cell lysates were prepared using the sample preparation kit (Proteinsimple) for an automated capillary Western blot system, JESS system (Proteinsimple). Cell lysates were diluted to the same protein concentrations using the 0.1× sample buffer (Proteinsimple) and mixed with 5× fluorescence master mix (Proteinsimple) according to protocol instructions. Samples were denatured at 95° C. following protocol instructions. JESS was run using a 1:400 diluted anti-GAA primary antibody (Abcam ab137068) diluted with milk-free antibody diluent; protein normalization substrate; horseradish peroxidase (HRP)-conjugated secondary antibodies; chemiluminescence substrate; and wash buffer dispensed into indicated wells of the assay plate. Samples were loaded in the indicated locations on the JESS plate in triplicate with the biotinylated ladder marker and the assay plate was placed in the JESS apparatus. Signal intensity (peak area) of the protein was normalized to the peak area of the total protein included in the capillary well using the protein normalization kit and analysis on Compass Software (Proteinsimple). Quantitative analysis of GAA protein bands was performed using the Compass Software (ProteinSimple). FIG. 9 and FIG. 10 show increases in GAA protein in patient iPSC-derived myotubes after treatment with selected PPMO compounds.

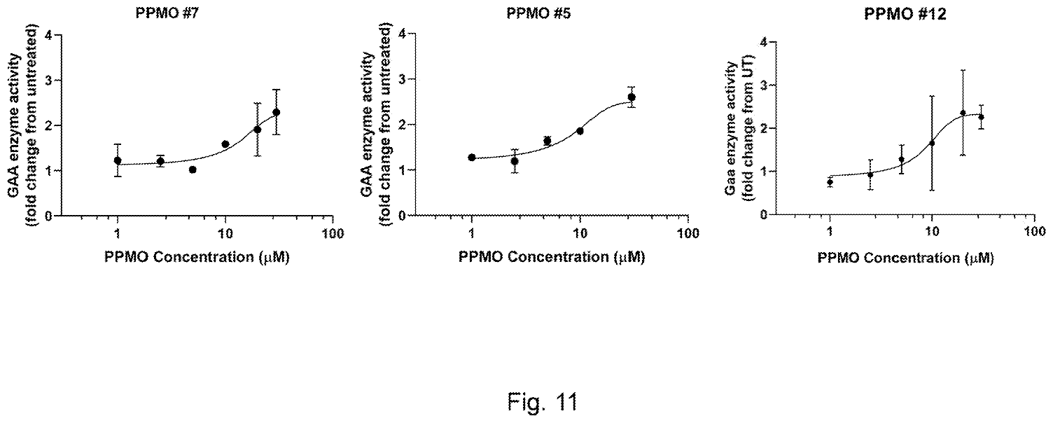

PPMO increase GAA protein in LOPD patient iPSC-derived myotubes. Patient iPSC-derived myoblasts were plated in 24-well collagen-coated plates (Thermo Fisher) at 80,000 cells/well in Expansion Media (EM, iXCells Biotechnologies). After 48 hours of growth in EM, cells were washed in PBS and media is changed to Differentiation Media (DM, iXCells Biotechnologies). Cells were incubated in DM for 48 hours, then treated with PPMO-supplemented DM and incubated without media changes for 4 days. For GAA Activity Assay lysis, cells were washed once with PBS and then lysed in ice-cold GAA Activity Assay Buffer (Abcam). FIG. 11 shows dose dependent increases in GAA enzyme activity in patient iPSC-derived myotubes after treatment with selected PPMO compounds.

### Example 8—Abasic Substitution Reduces PPMO Aggregation

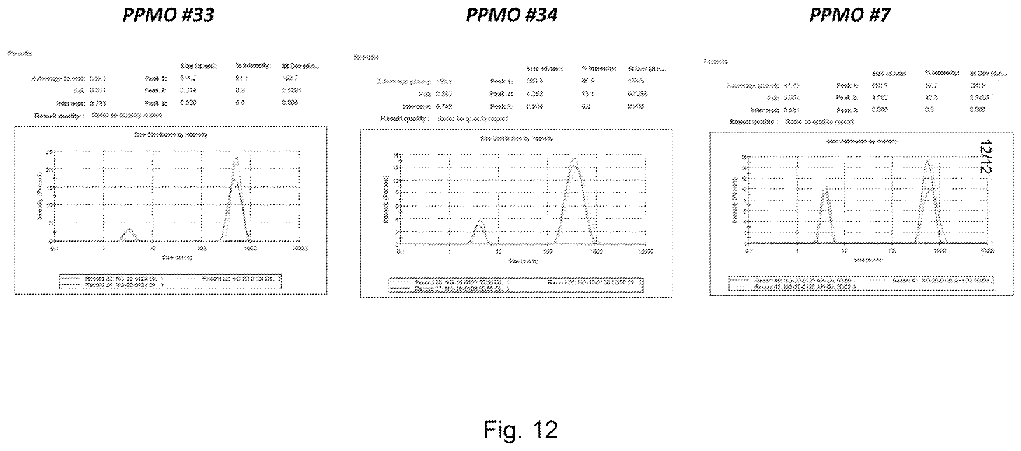

Aggregation of PPMO samples in constant concentration solutions in PBS (Gibco) were measured by dynamic light scattering using the Zetasizer Nano (Malvern) using the manufacture's standard protocol. FIG. 12 shows that abasic substitution reduces PPMO aggregation. The ratio of free PPMO increases with abasic substitution as measured by dynamic light scattering (DLS).

In summary, the PPMO compounds provided herein consistently corrected GAA splicing and increased GAA protein and enzyme activity levels in LOPD patient-derived myotubes. Target engagement of human IVS1-GAA was confirmed in a mouse model of LOPD. Surprisingly, substituting an abasic subunit is nearly as effective at restoring GAA enzyme active as the parental sequence (e.g., PPMO 7 vs PPMO 33). Interestingly, the DLS data point to some alteration of aggregation or secondary structure formation in these sequences by the inclusion of an abasic subunit.

What is claimed is:

1. A conjugate comprising a modified antisense oligonucleotide and a cell-penetrating peptide, wherein:
the modified antisense oligonucleotide is 18-40 subunits in length, comprising a targeting sequence complementary to a target region within intron 1 (SEQ ID NO: 1) of a pre-mRNA of human acid alpha-glucosidase (GAA) gene, wherein
the antisense oligonucleotide comprises a morpholino oligomer;
the antisense oligonucleotide is covalently linked to the cell-penetrating peptide;
each subunit of the antisense oligonucleotide comprises a nucleobase or is an abasic subunit, wherein each subunit is taken together in order from the 5′ end of the antisense oligonucleotide to the 3′ end of the antisense oligonucleotide form the targeting sequence;
at least one subunit is an abasic subunit; and
wherein the targeting sequence, except for the abasic subunit or subunits, is at least 80% complementary to the target region.

2. The conjugate of claim 1, wherein the target region comprises a sequence selected from the group consisting of SEQ ID NO: 2 (GAA-IVS1(-189-167)) and SEQ ID NO: 3 (GAA-IVS1(-80-24)).

3. The conjugate of claim 2, wherein the target region comprises the sequence set forth as SEQ ID NO: 2.

4. The conjugate of claim 2, wherein the target region comprises the sequence set forth as SEQ ID NO: 3.

5. The conjugate of claim 1 or 2, wherein the target region is selected from GAA-IVS1(-189-167), GAA-IVS1(-80-56), GAA-IVS1(-76-52), GAA-IVS1(-74-55), GAA-IVS1(-72-48), GAA-IVS1(-71-47), GAA-IVS1(-70-46), GAA-IVS1(-69-45), GAA-IVS1(-66-42), GAA-IVS1(-65-41), and GAA-IVS1(-49-24).

6. The conjugate of claim 1 or 5, wherein the target region is GAA-IVS1(-189-167).

7. The conjugate of claim 1 or 6, wherein the targeting sequence comprises the sequence CCA GAA GGA AXX XCG AGA AAA GC (SEQ ID NO: 4), wherein each X is independently selected from guanine (G) or is abasic (B), wherein at least one X is B.

8. The conjugate of claim 1 or 7, wherein the targeting sequence comprises a sequence selected from the group consisting of:
i)
SEQ ID NO: 5
(CCA GAA GGA AGG BCG AGA AAA GC);
ii)
SEQ ID NO: 6
(CCA GAA GGA AGB GCG AGA AAA GC);
iii)
SEQ ID NO: 7
(CCA GAA GGA ABG GCG AGA AAA GC);
iv)
SEQ ID NO: 8
(CCA GAA GGA AGB BCG AGA AAA GC);
v)
SEQ ID NO: 9
(CCA GAA GGA ABB GCG AGA AAA GC);
and
vi)
SEQ ID NO: 10
(CCA GAA GGA ABG BCG AGA AAA GC).

10. The conjugate of claim 1 or 5, wherein the target region is selected from the group consisting of GAA-IVS1(-80-56), GAA-IVS1(-76-52), GAA-IVS1(-74-55), GAA-IVS1(-72-48), GAA-IVS1(-71-47), GAA-IVS1(-70-46), GAA-IVS1(-69-45), GAA-IVS1(-66-42), GAA-IVS1(-65-41), and GAA-IVS1(-49-24).

11. The conjugate of claim 1 or 10, wherein the target region is selected from the group consisting of GAA-IVS1(-72-48), GAA-IVS1(-71-47), GAA-IVS1(-70-46), GAA-IVS1(-69-45), GAA-IVS1(-66-42), and GAA-IVS1(-65-41).

12. The conjugate of claim 1 or 11, wherein the targeting sequence comprises a sequence selected from the group consisting of:
i)
SEQ ID NO: 11
(CTC ACX XXX CTC TCA AAG CAG CTC T);
ii)
SEQ ID NO: 12
(ACT CAC XXX XCT CTC AAA GCA GCT C);
iii)
SEQ ID NO: 13
(CAC TCA CXX XXC TCT CAA AGC AGC T);
iv)
SEQ ID NO: 14
(GCA CTC ACX XXX CTC TCA AAG CAG C);
v)
SEQ ID NO: 15
(GCG GCA CTC ACX XXX CTC TCA AAG C);
vi)
SEQ ID NO: 16
(GGC GGC ACT CAC XXX XCT CTC AAA G);
wherein each X is independently selected from guanine (G) or is abasic (B), wherein at least one X is B.

13. The conjugate of claim 1 or 12, wherein the targeting sequence is selected from the group consisting of:
i)
SEQ ID NO: 17
(GCA CTC ACB GGG CTC TCA AAG CAG C);
ii)
SEQ ID NO: 18
(GCA CTC ACG BGG CTC TCA AAG CAG C);
iii)
SEQ ID NO: 19
(GCA CTC ACG GBG CTC TCA AAG CAG C);
iv)
SEQ ID NO: 20
(GCA CTC ACG GGB CTC TCA AAG CAG C);
v)
SEQ ID NO: 21
(GCA CTC ACB BGG CTC TCA AAG CAG C);
vi)
SEQ ID NO: 22
(GCA CTC ACG BBG CTC TCA AAG CAG C);
vii)
SEQ ID NO: 23
(GCA CTC ACG GBB CTC TCA AAG CAG C);
and
viii)
SEQ ID NO: 24
(GGC GGC ACT CAC GBB GCT CTC AAA G).

14. The conjugate of any one of claims 1 to 13, wherein the targeting sequence, except for the abasic subunit or subunits, is at least 84%, at least 88%, or at least 92% complementary to the target region.

15. The conjugate of any one of claims 1 to 13, wherein the targeting sequence, except for the abasic subunit or subunits, is at least 90% complementary to the target region.

16. The conjugate of any one of claims 1 to 5, wherein the targeting sequence, except for the abasic subunit or subunits, is at least 95% complementary to the target region.

17. The conjugate of any one of claims 1 to 5, wherein the targeting sequence, except for the abasic subunit or subunits, is 100% complementary to the target region.

18. The conjugate of any one of claims 1 to 4, wherein each abasic subunit is at least 8 subunits from the 5′ or 3′ end of the targeting sequence.

19. The conjugate of any one of claims 1 to 4, wherein the antisense oligonucleotide comprises 1 to 5 abasic subunits.

20. The conjugate of any one of claims 1 to 4 or 19, wherein the antisense oligonucleotide comprises 1, 2, 3, or 4 abasic subunits.

21. The conjugate of any one of claims 1 to 20, wherein the conjugate is a compound of Formula IV: 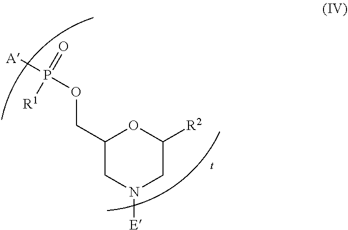
or a pharmaceutically acceptable salt thereof,
wherein:
A′ is selected from —N(H)CH₂C(O)NH₂, —N(C₁₋₆-alkyl)CH₂C(O)NH₂, 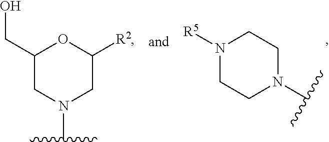
wherein
R⁵ is —C(O)(O-alkyl)ₓ-OH, wherein x is 3-10 and each alkyl group is, independently at each occurrence, C₂₋₆-alkyl,
or R⁵ is selected from H, —C(O)C₁₋₆-alkyl, trityl, monomethoxytrityl, —(C₁₋₆-alkyl)-R⁶, —(C₁₋₆-heteroalkyl)-R⁶, aryl-R⁶, heteroaryl-R⁶, —C(O)O—(C₁₋₆-alkyl)-R⁶, —C(O)O-aryl-R⁶, —C(O)O-heteroaryl-R⁶, and 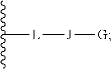
R⁶ is selected from OH, SH, and NH₂, or R⁶ is O, S, or NH, each of which is covalently linked to a solid support;
each R¹ is independently selected from OH and —N(R³)(R⁴), wherein each R³ and R⁴ are, independently at each occurrence, H or —C₁₋₆-alkyl;
each R² is independently, at each occurrence, selected from H (abasic), a nucleobase, and a nucleobase functionalized with a chemical protecting group, wherein the nucleobase, independently at each occurrence, comprises a C₃₋₆-heterocyclic ring selected from pyridine, pyrimidine, purine, and deaza-purine;
t is 8-40;
E′ is selected from H, —C₁₋₆-alkyl, —C(O)C₁₋₆-alkyl, benzoyl, stearoyl, trityl, monomethoxytrityl, dimethoxytrityl, trimethoxytrityl, 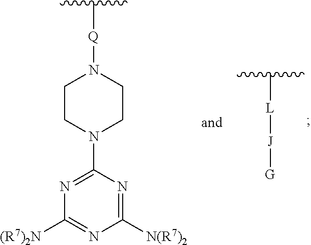
wherein
Q is —C(O)(CH₂)₆C(O)— or —C(O)(CH₂)₂S₂(CH₂)₂C(O)—;
R⁷ is —(CH₂)₂OC(O)N(R⁸)₂, wherein R⁸ is —(CH₂)₆NHC(═NH)NH₂;
L is selected from glycine, proline, W, W—W, or R⁹, wherein L is covalently linked by an amide bond to the N-terminus or C-terminus of J;
W is —C(O)—(CH₂)ₘ—NH—, wherein m is 2 to 12;
R⁹ is selected from the group consisting of: 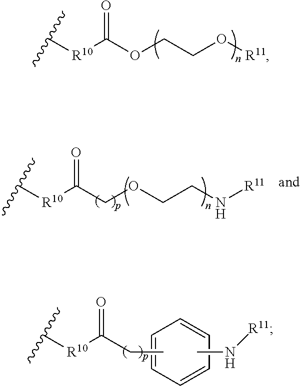
n is 1, 2, 3, 4, 5, 6, 7, 8, 9, or 10;
p is 2, 3, 4, or 5;
R¹⁰ is selected from a bond, glycine, proline, W, or W—W;
R¹¹ is selected from the group consisting of glycine, proline, W, W—W, and 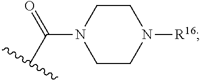
R¹⁶ is selected from a bond, glycine, proline, W, or W—W; wherein R¹⁶ is covalently linked by an amide bond to the N-terminus or C-terminus of J; J is a cell-penetrating peptide; and
G is selected from H, —C(O)C₁₋₆-alkyl, benzoyl, and stearoyl, wherein G is covalently linked to J;
provided that
A′ is 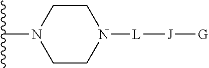
or E′ is

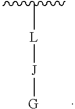

22. The conjugate of claim 21, wherein E′ is selected from H, —C₁₋₆-alkyl, —C(O)C₁₋₆-alkyl, benzoyl, stearoyl, trityl, monomethoxytrityl, dimethoxytrityl, trimethoxytrityl, and

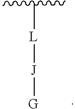

23. The conjugate of claim 21 or 22, wherein A′ is selected from —N(C₁₋₆-alkyl)CH₂C(O)NH₂,

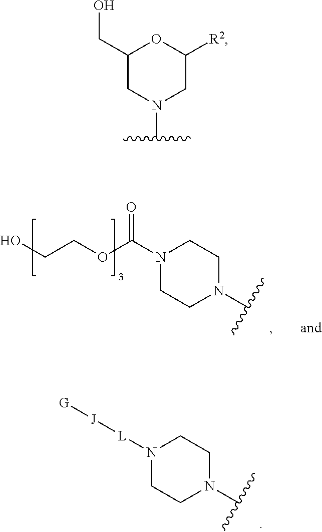

24. The conjugate of any one of claims 21-23, wherein E′ is selected from H, —C(O)CH₃, benzoyl, stearoyl, trityl, 4-methoxytrityl, and

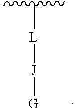

25. The conjugate of any one of claims 21-24, wherein A′ is selected from —N(C₁₋₆-alkyl)CH₂C(O)NH₂, 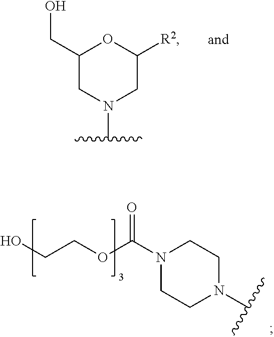
and E′ is

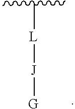

26. The conjugate of any one of claims 21-24, wherein A′ is 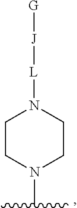
and E′ is selected from H, —C(O)CH₃, trityl, 4-methoxytrityl, benzoyl, and stearoyl.

27. The conjugate of claim 21, wherein the peptide-oligonucleotide conjugate of Formula IV is a peptide-oligonucleotide conjugate selected from: 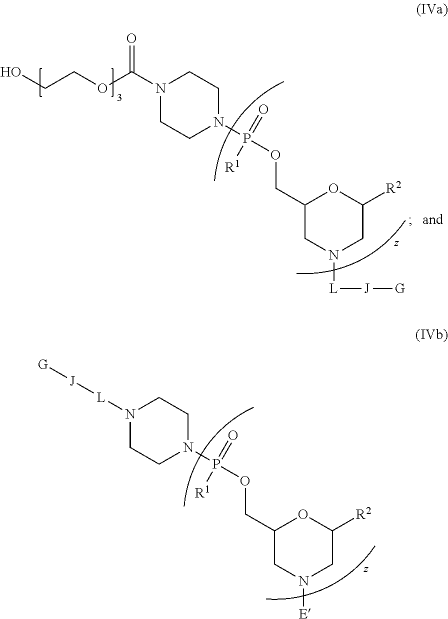
wherein E′ is selected from H, C₁₋₆-alkyl, —C(O)CH₃, benzoyl, and stearoyl.

28. The conjugate of any one of claims 21 to 27, wherein the conjugate is of the Formula (IVa).

29. The conjugate of any one of claims 21 to 27, wherein the conjugate is of the Formula (IVb).

30. The conjugate of any one of claims 21 to 27, wherein each R¹ is —N(CH₃)₂.

31. The conjugate of any one of claims 21 to 30, wherein each nucleobase, independently at each occurrence, is selected from adenine, guanine, cytosine, 5-methyl-cytosine, thymine, uracil, and hypoxanthine.

32. The conjugate of any one of claims 21 to 31, wherein the targeting sequence comprises the sequences:
i)
SEQ ID NO: 4
(CCA GAA GGA AXX XCG AGA AAA GC);
ii)
SEQ ID NO: 11
(CTC ACX XXX CTC TCA AAG CAG CTC T);
iii)
SEQ ID NO: 12
(ACT CAC XXX XCT CTC AAA GCA GCT C);
iv)
SEQ ID NO: 13
(CAC TCA CXX XXC TCT CAA AGC AGC T);
v)
SEQ ID NO: 14
(GCA CTC ACX XXX CTC TCA AAG CAG C);
vi)
SEQ ID NO: 15
(GCG GCA CTC ACX XXX CTC TCA AAG C);
vii)
SEQ ID NO: 16
(GGC GGC ACT CAC XXX XCT CTC AAA G);
wherein each X is independently selected from guanine (G) or is abasic (B), wherein at least one X is B.

33. The conjugate of any one of claims 21 to 33, wherein the targeting sequence comprises a sequence selected from the group consisting of:
i)
SEQ ID NO: 5
(CCA GAA GGA AGG BCG AGA AAA GC );
ii)
SEQ ID NO: 6
(CCA GAA GGA AGB GCG AGA AAA GC);
iii)
SEQ ID NO: 7
(CCA GAA GGA ABG GCG AGA AAA GC);
iv)
SEQ ID NO: 8
(CCA GAA GGA AGB BCG AGA AAA GC);
v)
SEQ ID NO: 9
(CCA GAA GGA ABB GCG AGA AAA GC);
vi)
SEQ ID NO: 10
(CCA GAA GGA ABG BCG AGA AAA GC);
vii)
SEQ ID NO: 17
(GCA CTC ACB GGG CTC TCA AAG CAG C);
viii)
SEQ ID NO: 18
(GCA CTC ACG BGG CTC TCA AAG CAG C);
ix)
SEQ ID NO: 19
(GCA CTC ACG GBG CTC TCA AAG CAG C);
x)
SEQ ID NO: 20
(GCA CTC ACG GGB CTC TCA AAG CAG C);
xi)
SEQ ID NO: 21
(GCA CTC ACB BGG CTC TCA AAG CAG C);
xii)
SEQ ID NO: 22
(GCA CTC ACG BBG CTC TCA AAG CAG C);
xiii)
SEQ ID NO: 23
(GCA CTC ACG GBB CTC TCA AAG CAG C);
and
xiv)
SEQ ID NO: 24
(GGC GGC ACT CAC GBB GCT CTC AAA G).

34. The conjugate of any one of claims 21 to 33, wherein L is glycine.

35. The conjugate of any one of claims 21 to 33, wherein L is proline.

36. The conjugate of any one of claims 21 to 33, wherein L is —C(O)—(CH₂)₅—NH—.

37. The conjugate of any one of claims 21 to 33, wherein L is —C(O)—(CH₂)₂—NH—.

38. The conjugate of any one of claims 21 to 33, wherein L is —C(O)—(CH₂)₂—NH—C(O)—(CH₂)₅—NH—.

39. The conjugate of any one of claims 21 to 33, wherein L is 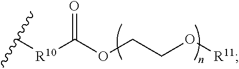
R¹⁰ is a bond; and R¹¹ is selected from: glycine and

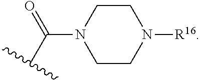

40. The conjugate of any one of claims 21 to 33, wherein L is 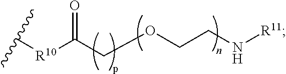
R¹⁰ is a bond; and R¹¹ is selected from: glycine and

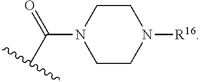

41. The conjugate of any one of claims 21 to 33, wherein L is 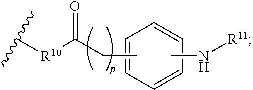
R¹⁰ is a bond; and R¹¹ is selected from: glycine and

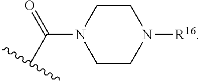

42. The conjugate of any one of claims 21 to 39, wherein J is selected from rTAT, TAT, R₉F₂, R₅F₂R₄, R₄, R₅, R₆, R₇, R₈, R₉, (RXR)₄, (RXR)₅, (RXRRBR)₂, (RAR)₄F₂, (RGR)₄F₂.

43. The conjugate of any one of claims 21 to 42, wherein G is selected from H, C(O)CH₃, benzoyl, and stearoyl.

44. The conjugate of any one of claims 21 to 43, wherein G is H or —C(O)CH₃.

45. The conjugate of any one of claims 21 to 44, wherein G is H.

46. The conjugate of any one of claims 21 to 44, wherein G is —C(O)CH₃.

47. A pharmaceutical composition comprising the conjugate of any one of claims 1 to 46, or a pharmaceutically acceptable salt thereof, and at least one pharmaceutically acceptable carrier.

48. A method of treating a disease in a subject in need thereof, the method comprising administering a therapeutically effective amount of the conjugate of any one of claims 1 to 46 or the pharmaceutical composition of claim 47 to the subject.

49. The method of claim 48, wherein the disease is Pompe disease.

50. The method of claim 48, where the subject is a human.

51. The method of claim 50, wherein the human is a child.

52. The method of claim 50, wherein the human is an adult.

53. An antisense oligomer compound selected from: 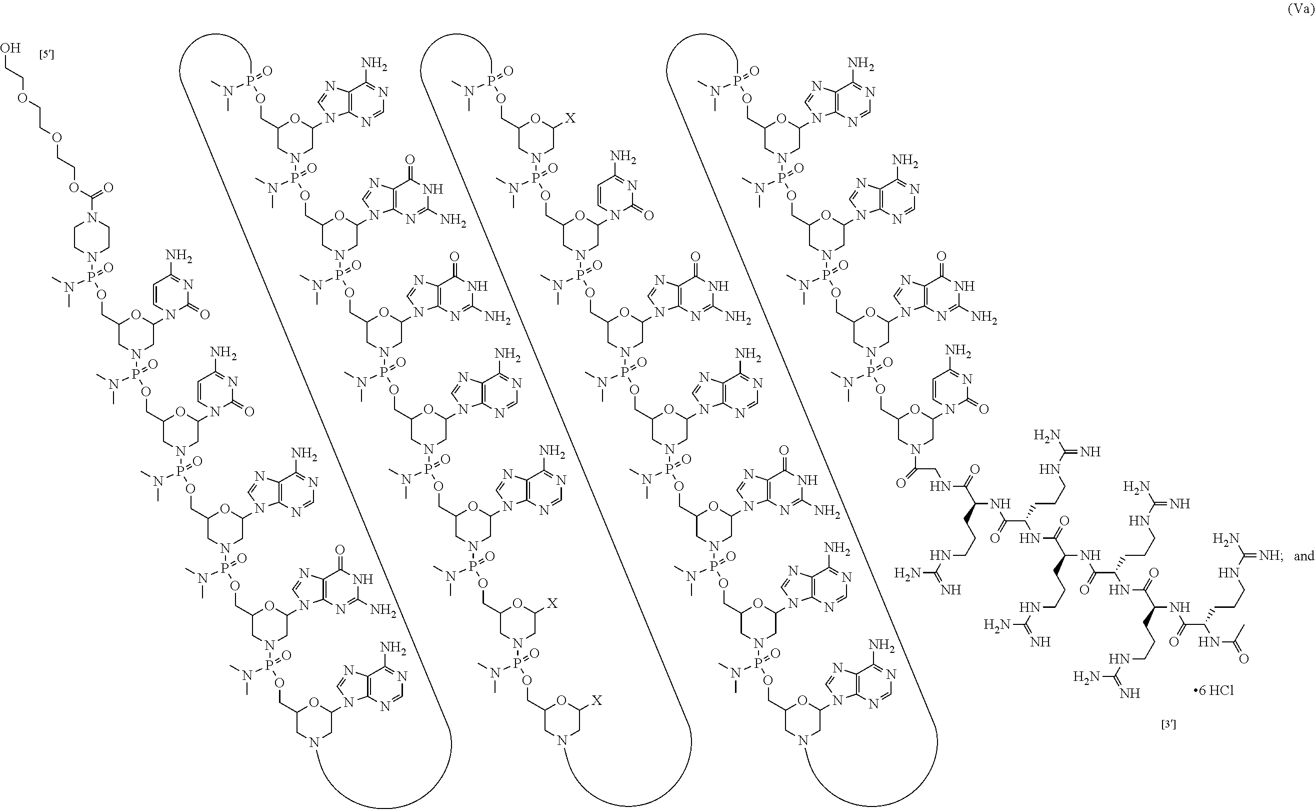 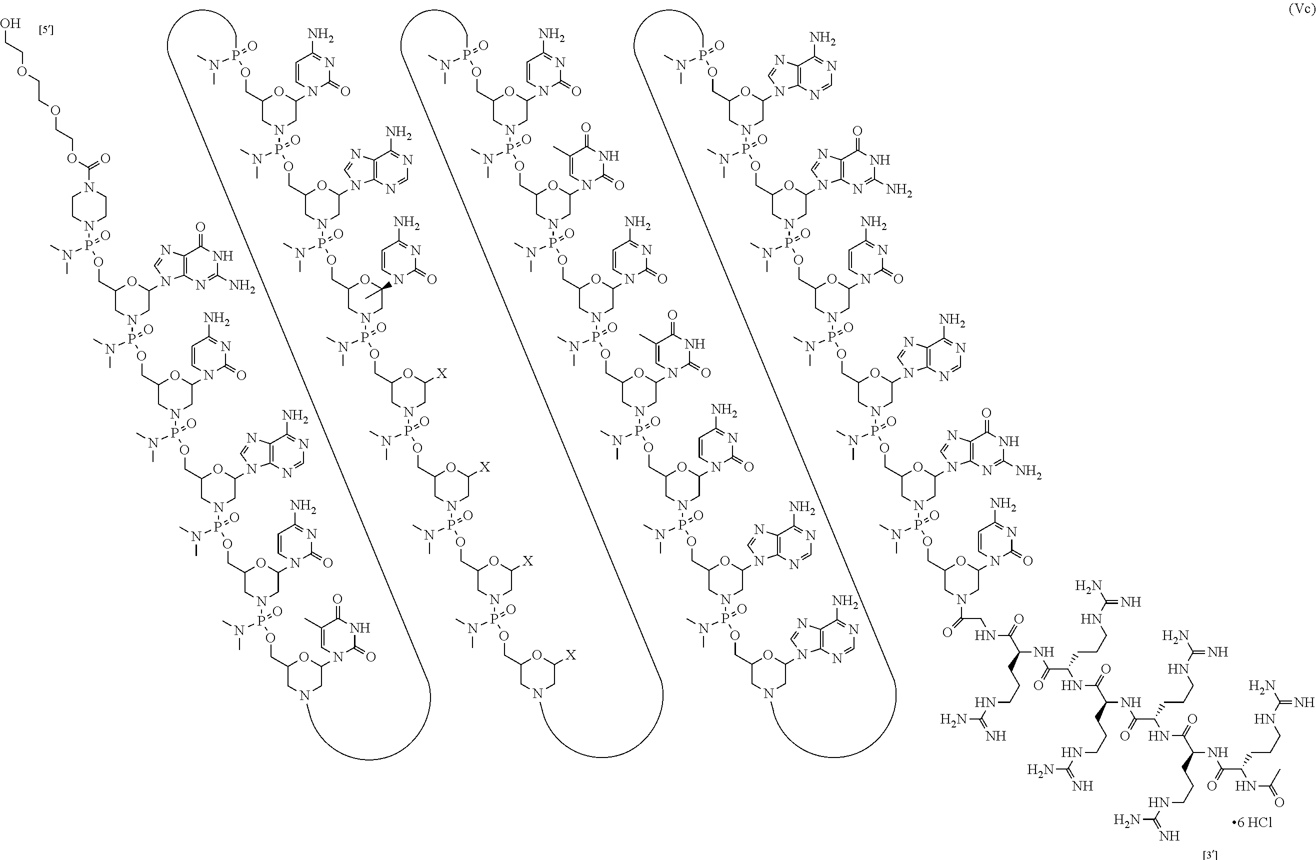
wherein each X is independently selected from guanine (G) or is abasic (B), wherein at least one X is B.

54. The antisense oligomer compound of claim 53, wherein the antisense oligomer compound is: 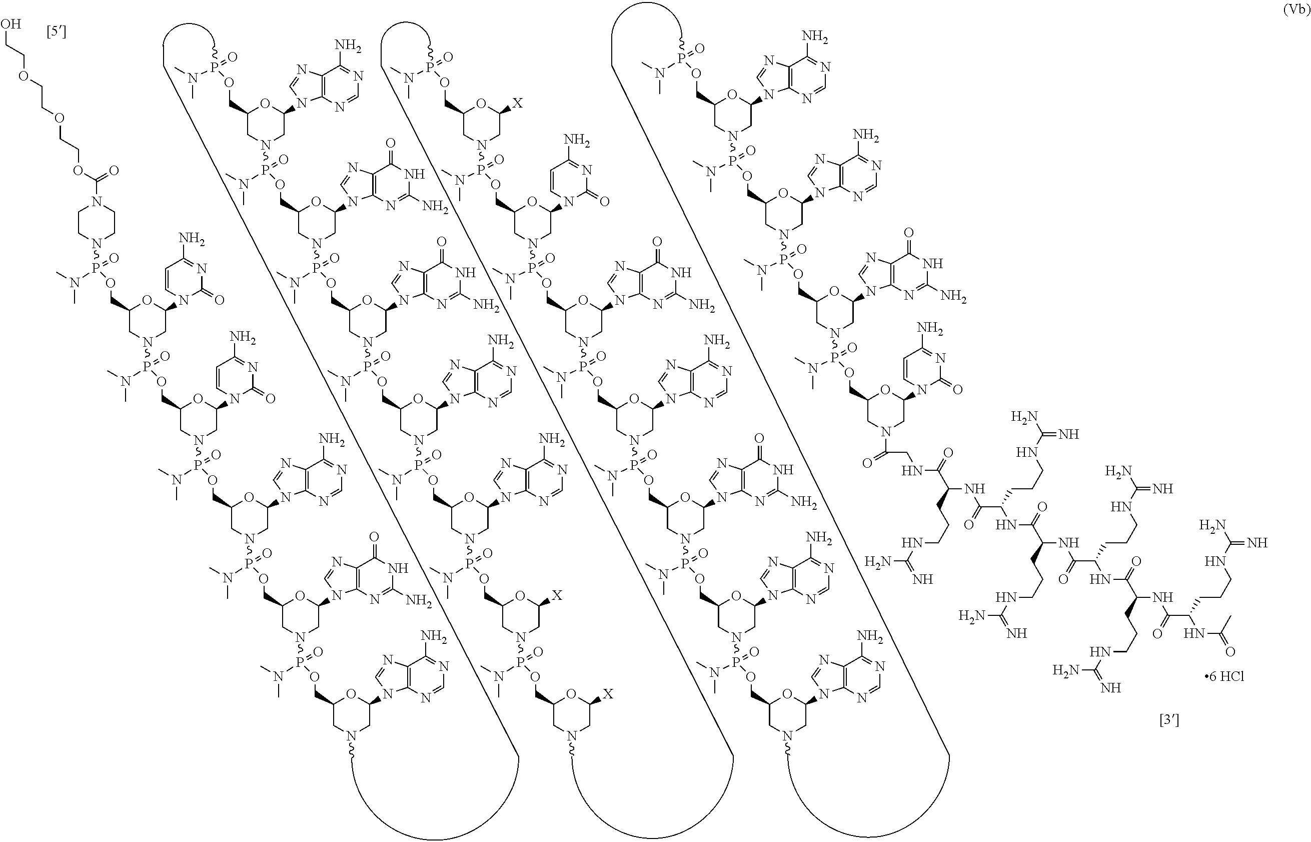
wherein each X is independently selected from guanine (G) or is abasic (B), wherein at least one X is B.

55. The antisense oligomer compound of claim 53, wherein the antisense oligomer compound is: 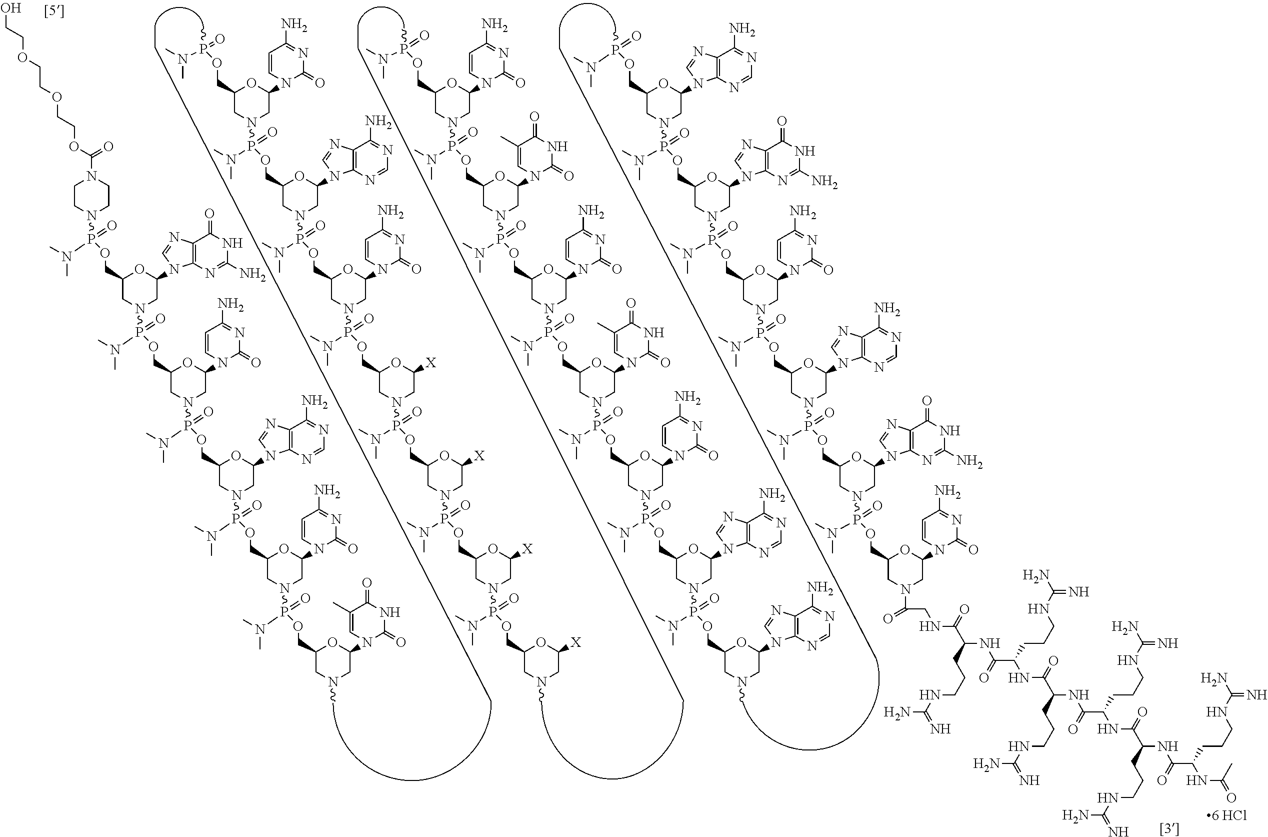
wherein each X is independently selected from guanine (G) or is abasic (B), wherein at least one X is B.
In an embodiment, B is H.
